(19) United States
(12) Patent Application Publication
(10) Pub. No.: US 2010/0297023 A1
(43) Pub. Date: Nov. 25, 2010

(54) Lipid

(75) Inventors: Andrew David Miller, Chiswick (GB); Patrick Arbuthnot, Wits (ZA); Michael Jorgensen, London (GB); Michael Keller, Basel (CH); Nazila Kamaly, London (GB); Abderrahim Aissaoui, London (GB); Maya Thanou, London (GB)

Correspondence Address: FOLEY AND LARDNER LLP;SUITE 500, 3000 K STREET NW, WASHINGTON, DC, 20007, US

(73) Assignee: IMUTHES LIMITED UNIVERSITY OF THE WITWATERSRAND

(21) Appl. No.: 12/520,261

(22) PCT Filed: Dec. 21, 2007
(86) PCT No.: PCT/GB07/04967

§ 371 Date: Apr. 20, 2010

(30) Foreign Application Priority Data
Dec. 22, 2006 (ZA) 2006/07957
Aug. 22, 2007 (ZA) 2007/07053
Dec. 11, 2007 (GB) 0724193.8

(51) Int. Cl. A61K 47/18; C07C 237/22; A61K 31/713; A61K 48/00; A61K 49/10; C12N 5/02

(52) U.S. Cl. 424/9.3; 564/159; 514/44 A; 514/44 R; 514/788; 435/375; 428/402.2

(57) ABSTRACT

The present invention provides a lipid of the formula (I) R₃R₄N—[Y]_{q}—(CₚH₂ₚ)—X-Linker-NR₁R₂ (I) wherein R₃ and R₄ are independently selected from H and hydrocarbyl groups; q is an integer selected from 1 to 10; Y represents a group (CₙH₂ₙ)NR₅, wherein (i) when q is 1, n is 2, or (ii) when q is greater than 1, each Y may be the same or different and each n is an integer independently selected from 1 to 10, with the proviso that for at least one unit Y, n is 2, and (iii) each R₅ is independently selected from H and hydrocarbyl groups; p is an integer selected from 1 to 10; X is an optional group selected from —NR₇—, —C(═O)—NR₈—, —NR₉—C(═O)—, —C(═O)—, —O—, and NR₁₀—C(=0)0-, wherein each R₇, R₈, R₉ and R₁₀ is independently selected from H and hydrocarbyl groups, Linker is an optional group selected from amino acid residues, peptide residues and groups of the formula —(OCH₂CH₂)₁₋₁₀, —NR₆-(CvH₂ᵥ)—C(═O)—, wherein R₆ is H or a hydrocarbyl group and v is an integer selected from 1 to 11, and —C(=0)-(CH₂)₀₋₁₀—CH₂—C(=0)-; R₁ is selected from acyclic groups having from 4 to 30 carbon atoms; and R₂ is selected from H and acyclic groups having from 4 to 30 carbon atoms. Said lipids can be used in a delivery vehicle (e.g. micelle or liposome) for the delivery of one or more (therapeutic or diagnostic) agents.

### FIELD OF INVENTION

The present invention relates to a lipid. In particular, the invention relates to a lipid that can be used in a delivery vehicle for the in vitro or in vivo delivery of one or more agents. The present invention also relates to use of the lipid or delivery vehicle in therapy, and a method for delivery of one or more agents using the lipid.

### BACKGROUND TO THE INVENTION

Methods for viral DNA delivery suffer from many problems including immune responses, inability to deliver viral DNA vectors repeatedly, difficulty in generating high viral titres, and the possibility of infectious virus. Non-viral delivery methods provide an alternative system that is devoid of these problems and has therefore prompted the development of less hazardous, non-viral approaches to gene transfer.

The ability of these non-viral delivery systems to deliver their cargo is dependent on several factors—such as loading of the cargo, access to the target organ, association with the targeted cells and intracellular trafficking to access to the proper cellular compartment. The most challenging drugs for these systems are polynucleotides as they are difficult to load and it is difficult for them to get into the cytoplasm of a cell.

Among the possible delivery systems, cationic liposomes have gained widespread attention as they are simple to produce, allow for efficient compaction of the polynucleotides and efficient delivery to cells in vitro. In vitro studies have shown that different mixtures of cationic lipids and neutral lipids give very efficient delivery of polynucleotides. However, this efficiency is usually limited to artificial media not containing serum or other components found in in vivo biological fluids.

The negatively charged proteins present in the in vivo media bind to the cationic delivery system modifying the size or other structural properties of the liposomes, reducing their activity and interaction with cells and ultimately resulting in their clearance from the body. These effects are regarded as one of the major limiting factors for the use of cationic liposomes in vivo. Therefore, it is important to optimise the formulations of lipids in order to minimise interaction with proteins while maintaining cell-binding and delivery properties.

Our publication WO 97/45442 teaches lipids useful in the above systems. A preferred lipid of WO 97/45442 is N¹-cholesteryloxycarbonyl-3,7-diazanonane-1,9-diamine (CDAN). Other teaching is provided in the art of useful lipids, for example that of U.S. Pat. No. 5,171,678.

The present invention seeks to provide further improvements in non-viral delivery. The present invention also seeks to provide lipids which may be useful in therapy or diagnosis.

### SUMMARY OF THE INVENTION

Aspects of the present invention are presented in the accompanying claims.

The present invention provides a lipid of the formula (I)

R₃R₄N—[Y]_{q}(CₚH₂ₚ)—X-Linker-NR₁R₂ (I)

wherein
R₃ and R₄ are independently selected from H and hydrocarbyl groups;
q is an integer selected from 1 to 10;
Y represents a group (CₙH₂ₙ)NR₅, wherein (i) when q is 1, n is 2, or (ii) when q is greater than 1, each Y may be the same or different and each n is an integer independently selected from 1 to 10, with the proviso that for at least one unit Y, n is 2, and (iii) each R₅ is independently selected from H and hydrocarbyl groups;
p is an integer selected from 1 to 10;
X is an optional group selected from —NR₇—, —C(═O)—NR₈—, —NR₉—C(═O)—, —C(═O)—, —O—, and —NR₁₀—C(═O)O—, wherein each R₇, R₈, R₉ and R₁₀ is independently selected from H and hydrocarbyl groups,

Linker is an optional group selected from amino acid residues, peptide residues and groups of the formulae —(OCH₂CH₂)₁₋₁₀, —NR₆—(CᵥH₂ᵥ)—C(═O)—, wherein R₆ is H or a hydrocarbyl group and v is an integer selected from 1 to 11, and —C(═O)—(CH₂)₀₋₁₀—CH₂—C(═O)—;

R₁ is selected from acyclic groups having from 4 to 30 carbon atoms; and
R₂ is selected from H and acyclic groups having from 4 to 30 carbon atoms.

The present invention also provides micelles and liposomes formed from or comprising the lipid, delivery vehicles comprising the lipid in combination with one or more agents, and their methods of preparation.

In further aspects, the present invention provides the lipid, micelle, liposome or delivery vehicle for use in therapy or diagnosis, in particular for the treatment of a (genetic) disorder or condition or disease.

The present invention also provides a method for of delivery of one or more agents to cells and a method of delivering one or more agents to one or more cells, comprising using the lipid of the present invention.

The lipids described herein have a number of advantages. These advantages will be apparent in the following description. By way of example, the present invention is advantageous since it provides a method for delivering agents, such as siRNA, in vivo and in vitro mediated by non-viral methods.

By way of further example, the present invention is advantageous since the lipids described herein and micelles, liposomes and delivery vehicles formed therefrom can provide enhanced activity without any impairment in long term circulating properties.

By way of further example, the present invention is advantageous since the lipids described herein and micelles, liposomes and delivery vehicles formed therefrom can be used to deliver one or more agents to a cell, tissue or organ that is or is derivable (preferably, derived) from the liver, a Kupffer cell, an endothelial cell, a sinusoidal endothelial cell, a tumour, a tumour endothelial cell, tumour vasculature, angiogenic tumour vasculature or a microvessel.

By way of further example, the present invention is advantageous since the lipids/micelles/liposomes/delivery vehicles can have a higher loading capacity for one or more agents than the liposomes of the prior art.

### DETAILED DESCRIPTION OF THE INVENTION

The present invention provides a lipid of the formula (I)

R₃R₄N—[Y]_{q}—(CₚH₂ₚ)—X-Linker-NR₁R₂ (I)

wherein
R₃ and R₄ are independently selected from H and hydrocarbyl groups;
q is an integer selected from 1 to 10;
Y represents a group (CₙH₂ₙ)NR₅, wherein (i) when q is 1, n is 2, or (ii) when q is greater than 1, each Y may be the same or different and each n is an integer independently selected from 1 to 10, with the proviso that for at least one unit Y, n is 2, and (iii) each R₅ is independently selected from H and hydrocarbyl groups;
p is an integer selected from 1 to 10;
X is an optional group selected from —NR₇—, —C(═O)—NR₈—, —NR₉—C(═O)—, —C(═O)—, —O—, and —NR₁₀—C(═O)O—, wherein each R₇, R₈, R₉ and R₁₀ is independently selected from H and hydrocarbyl groups,

Linker an optional group selected from amino acid residues, peptide residues and groups of the formulae —(OCH₂CH₂)₁₋₁₀, —NR₆—(CᵥH₂ᵥ)—C(═O)—, wherein R₆ is H or a hydrocarbyl group and v is an integer selected from 1 to 11, and —C(═O)—(CH₂)₀₋₁₀—CH₂—C(═O)—;

R₁ is selected from acyclic groups having from 4 to 30 carbon atoms; and
R₂ is selected from H and acyclic groups having from 4 to 30 carbon atoms.

As used herein, “independently selected from” should be understood to mean that each number or moiety may be the same or different and each is independently selected from the alternatives given.

The term “hydrocarbyl group” means a group comprising at least C and H and may optionally comprise one or more other suitable substituents. Examples of such substituents may include halo, alkoxy, nitro, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group.

By way of example, the hydrocarbyl group may be selected from C₁-C₁₀ hydrocarbyl, C₁-C₅ hydrocarbyl or C₁-C₃ hydrocarbyl.

A typical hydrocarbyl group is a hydrocarbon group. Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

By way of example, the hydrocarbon group may be selected from C₁-C₁₀ hydrocarbon, C₁-C₅ hydrocarbon or C₁-C₃ hydrocarbon. For example, the hydrocarbon group may be an alkyl group, such as C₁-C₁₀ alkyl, C₁-C₅ alkyl or C₁-C₃ alkyl.

The hydrocarbyl/hydrocarbon/alkyl may be straight chain or branched and/or may be saturated or unsaturated.

In one embodiment the hydrocarbyl/hydrocarbon/alkyl may be selected from straight or branched hydrocarbon groups containing at least one heteroatom in the group.

In one embodiment the hydrocarbyl/hydrocarbon/alkyl may be a hydrocarbyl group comprising at least two carbons or wherein the total number of carbons and heteroatoms is at least two.

In one embodiment the hydrocarbyl/hydrocarbon/alkyl may be selected from hydrocarbyl groups, preferably straight or branched hydrocarbon groups, containing at least one heteroatom in the group. Preferably the heteroatom is selected from sulphur, nitrogen or oxygen. Thus, the hydrocarbyl/hydrocarbon/alkyl may be selected from straight or branched alkyl groups, preferably C₁₋₁₀ alkyl, more preferably C₁₋₅ alkyl, containing at least one heteroatom in the group. Preferably the heteroatom is selected from sulphur, nitrogen and oxygen. Preferably, the alkyl is selected from straight chain alkyl groups.

In another embodiment, the hydrocarbyl/hydrocarbon/alkyl may be selected from straight or branched alkyl groups, preferably C₁₋₁₀ alkyl, more preferably C₁₋₅ alkyl. Preferably, the alkyl is selected from straight chain alkyl groups.

q

According to the present invention, q is an integer independently selected from 1 to 10.

In one preferred aspect q is an integer independently selected from 1 to 4, such as from 2 and 3.

In one preferred aspect, q is 2.

n

According to the present invention, n is an integer independently selected from 1 to 10. When q is 1, n is 2, i.e. Y represents a group C₂H₄. When q is greater than 1, each Y may be the same or different and each n is independently selected from an integer between 1 and 10, with the proviso that at least one n is 2.

In one preferred aspect, when q is greater than 1, each n is an integer independently selected from 2 to 6, such as from 2 to 4 or 2 and 3.

As discussed herein at least one n is 2. In one aspect, at least two n are 2. In one embodiment, each n is 2.

Preferably, each group CₙH₂ₙ is a group (CH₂)ₙ, and preferred n are as described above. For example, when n is 3, the group CₙH₂ₙ is a group CH₂—CH₂—CH₂.

p

According to the present invention, p is an integer independently selected from 1 to 10.

In one preferred aspect p is an integer independently selected from 1 to 6, such as from 2 and 3.

In one preferred aspect, p is 2.

### R₃, R₄ and R₅

R₃, R₄ and R₅ are independently selected from H and hydrocarbyl groups. In one preferred aspect each R₃, R₄ and R₅ is independently selected from H and C₁₋₁₀ alkyl groups. For example each R₃, R₄ and R₅ may be independently selected from C₁₋₁₀ alkyl groups.

In one preferred aspect each R₃, R₄ and R₅ is independently selected from H and C₁₋₅ alkyl groups. For example each R₃, R₄ and R₅ may be independently selected from C₁₋₅ alkyl groups.

In one preferred aspect each R₃, R₄ and R₅ is independently selected from H and C₁₋₃ alkyl groups. For example each R₃, R₄ and R₅ may be independently selected from C₁₋₃ alkyl groups.

In one preferred aspect each R₃, R₄ and R₅ is independently selected from H and methyl.

In one preferred aspect
R₃ is methyl or R₃ is H; and/or
R₄ is methyl or R₄ is H; and/or
R₅ is methyl or R₅ is H.

It will be understood that each R₃, R₄ and R₅ may be the same or different. In one preferred aspect each R₃, R₄ and R₅ are the same.

In preferred aspects the present lipid is selected from lipids of the formulae

H₂N—(CH₂)₂—HN—(CH₂)₂—X-Linker-NR₁R₂

H₂N—(CH₂)₃—HN—(CH₂)₂—X-Linker-NR₁R₂

H₂N—(CH₂)₄—HN—(CH₂)₂—X-Linker-NR₁R₂

H₂N—(CH₂)₂—HN—(CH₂)₃—X-Linker-NR₁R₂

H₂N—(CH₂)₂—HN—(CH₂)₃—HN—(CH₂)₂—X-Linker-NR₁R₂

H₂N—(CH₂)₂—HN—(CH₂)₃—HN—(CH₂)₃—X-Linker-NR₁R₂

H₂N—(CH₂)₂—HN—(CH₂)₄—HN—(CH₂)₂—X-Linker-NR₁R₂

H₂N—(CH₂)₃—HN—(CH₂)₂—HN—(CH₂)₂—X-Linker-NR₁R₂

H₂N—(CH₂)₃—HN—(CH₂)₄—HN—(CH₂)₂—X-Linker-NR₁R₂

H₂N—(CH₂)₂—HN—(CH₂)₃—HN—(CH₂)₄—HN—(CH₂)₃—X-Linker-NR₁R₂

Me₂N—(CH₂)₂—NMe—(CH₂)₂—X-Linker-NR₁R₂

Me₂N—(CH₂)₃—NMe—(CH₂)₂—X-Linker-NR₁R₂

Me₂N—(CH₂)₄—NMe—(CH₂)₂—X-Linker-NR₁R₂

Me₂N—(CH₂)₂—NMe—(CH₂)₃—X-Linker-NR₁R₂

Me₂N—(CH₂)₂—NMe—(CH₂)₃—NMe—(CH₂)₂—X-Linker-NR₁R₂

Me₂N—(CH₂)₂—NMe—(CH₂)₃—NMe—(CH₂)₃—X-Linker-NR₁R₂

Me₂N—(CH₂)₂—NMe—(CH₂)₄—NMe—(CH₂)₂—X-Linker-NR₁R₂

Me₂N—(CH₂)₃—NMe—(CH₂)₂—NMe—(CH₂)₂—X-Linker-NR₁R₂

Me₂N—(CH₂)₃—NMe—(CH₂)₄—NMe—(CH₂)₂—X-Linker-NR₁R₂

Me₂N—(CH₂)₂—NMe—(CH₂)₃—NMe—(CH₂)₄—NMe—(CH₂)₃—X-Linker-NR₁R₂

In preferred aspects the present lipid is selected from lipids of the formulae

H₂N—(CH₂)₂—HN—(CH₂)₃—HN—(CH₂)₂—X-Linker-NR₁R₂

Me₂N—(CH₂)₂—NMe—(CH₂)₃—NMe—(CH₂)₂—X-Linker-NR₁R₂

### X

X is an optional group selected from —NR₇—, —C(═O)—NR₈—, —NR₉—C(═O)—, —C(═O)—, —O—, and —NR₁₀—C(═O)O—, wherein R₇, R₈, R₉ and R₁₀ are selected from H and hydrocarbyl groups.

In one aspect group X is present and there is provided a lipid of the formula R₃R₄N—[Y]_{q}—(CₚH₂ₚ)—X-Linker-NR₁R₂. In another embodiment, group X is not present and there is provided a lipid of the formula R₃R₄N—[Y]_{q}—(CₚH₂ₚ)-Linker-NR₁R₂.

In aspects of the invention
X is —NR₇—
X is —C(═O)—NR₈—
X is —NR₉—C(═O)—
X is C(═O)—
X is —O—
X is —NR₁₀—C(═O)O—

In a preferred aspect X is —C(═O)—.

In another embodiment, R₇, R₈, R₉ and R₁₀ are selected from H and C₁₋₁₀ alkyl groups. For example R₇, R₈, R₉ and R₁₀ are selected from C₁₋₁₀ alkyl groups. In one preferred aspect R₇, R₈, R₉ and R₁₀ are selected from H and C₁₋₅ alkyl groups. For example R₇, R₈, R₉ and R₁₀ are selected from C₁₋₅ alkyl groups. In one preferred aspect R₇, R₈, R₉ and R₁₀ are selected from H and C₁₋₃ alkyl groups. For example R₇, R₈, R₉ and R₁₀ are selected from C₁₋₃ alkyl groups. In one preferred aspect R₇, R₈, R₉ and R₁₀ are selected from H and methyl.

### Linker

In one aspect the linker of the present lipid is optional. That is, in one aspect the present invention provides a lipid of the formula R₃R₄N—[Y]_{q}—(CₚH₂ₚ)—X—NR₁R₂.

When present, Linker is a group selected from amino acid residues, peptide residues and groups of the formulae —(OCH₂CH₂)₁₋₁₀, —NR₆—(CᵥH₂ᵥ)—C(═O)—, wherein R₆ is H or a hydrocarbyl group and v is an integer selected from 1 to 11, and —C(═O)—(CH₂)₀₋₁₀—CH₂—C(═O)—.

When the linker group is a peptide residue preferably it contains from 2 to 10 amino acids which may the same or different.

In one preferred aspect the linker group is an amino acid residue. It will be understood that by amino acid residue it meant an amino acid radical capable of linking R₃R₄N—[Y]_{q}—(CₚH₂ₚ)—X— and —NR₁R₂.

Suitable amino acid residues include those derived from the natural amino acids (L-α-amino acids).

Preferred amino acids from which the amino acid residue is derived include water soluble amino acids such as Arg, Lys, Ser and Thr. When the linker is a peptide residue preferred amino acids units include water soluble amino acids such as Arg, Lys, Ser and Thr.

In another embodiment, the amino acid residue is preferably a glycine residue.

In one aspect when the group Linker is an amino acid residue X is preferably present and is a —C(═O)— group. In a further aspect when the group Linker is an amino acid residue or a peptide residue X is preferably present and is a —C(═O)— group.

In another preferred embodiment, Linker is a group of formula —NR₆—(CᵥH₂ᵥ)—C(═O)—, wherein R₆ is H or a hydrocarbyl group and v is an integer selected from 1 to 11. In one embodiment, Linker is a group of formula —NH—CH₂—(CH₂)₀₋₁₀—C(═O)— in particular a group of formula —NH—CH₂—C(═O)— (i.e. a glycine residue).

In one aspect, Linker is a group of formula —NR₆—(CᵥH₂ᵥ)—C(═O)— as described above, in particular a glycine residue, and X is present and is a —C(═O)— group.

### R₁ and R₂

As discussed herein R₁ is selected from acyclic groups having from 4 to 30 carbon atoms and R₂ is selected from H and acyclic groups having from 4 to 30 carbon atoms.

In one aspect R₁ is selected from alkyl, alkenyl and alkynyl groups having from 4 to 30 carbon atoms and R₂ is selected from H and alkyl, alkenyl and alkynyl groups having from 4 to 30 carbon atoms. Preferably R₁ and R₂ are each independently selected from alkyl, alkenyl and alkynyl groups having from 4 to 30 carbon atoms.

In one aspect R₁ is selected from alkyl, alkenyl and alkynyl groups having from 12 to 30 carbon atoms and R₂ is selected from H and alkyl, alkenyl and alkynyl groups having from 12 to 30 carbon atoms. Preferably R₁ and R₂ are each independently selected from alkyl, alkenyl and alkynyl groups having from 12 to 30 carbon atoms.

In one aspect R₁ is selected from alkyl, alkenyl and alkynyl groups having 18 carbon atoms and R₂ is selected from H and alkyl, alkenyl and alkynyl groups having 18 carbon atoms. Preferably R₁ and R₂ are each independently selected from alkyl, alkenyl and alkynyl groups having 18 carbon atoms.

In one aspect R₁ is selected from alkyl groups having 18 carbon atoms and R₂ is selected from H and alkyl groups having 18 carbon atoms. Preferably R₁ and R₂ are each independently selected from alkyl groups having 18 carbon atoms.

### Preferred Lipids

In a preferred aspect of the invention the lipid is of the formula R₃R₄N—[Y]_{q}—(CₚH₂ₚ)—C(═O)—NR₆—(CᵥH₂ᵥ)—C(═O)—NR₁R₂, wherein definitions are as provided above.

In a preferred aspect of the invention the lipid is N′,N′-dioctadecyl-N-4,8-diaza-10-aminodecanoylglycine amide (“DODAG”):

H₂N—(CH₂)₂—HN—(CH₂)₃—HN—(CH₂)₂—C(═O)NH—CH₂—C(═O)—N[(CH₂)₁₇CH₃]₂

i.e. having the following structure

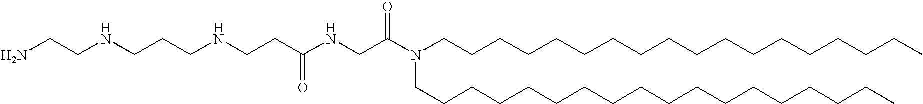

This lipid compound can be seen as comprising the head group of N¹-cholesteryloxycarbonyl-3,7-diazanonane-1,9-diamine (“CDAN”) and the “tail group” of dioctadecylglycyl spermine (“DOGS”). Hence an alternative name for “DODAG” is “CDAN-DOGS”.

In another preferred aspect of the invention the lipid is of the formula

Me₂N—(CH₂)₂—NMe—(CH₂)₃—NMe—(CH₂)₂—C(═O)NH—CH₂—C(═O)—N[(CH₂)₁₇CH₃]₂

i.e. having the following structure

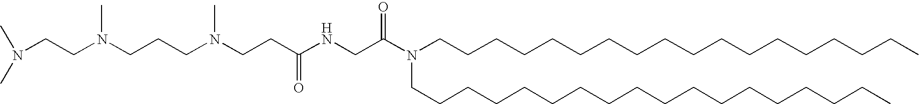

### Micelle

The lipid of the present invention may be used to form a micelle. Such micelles are usually an aggregate of the lipid molecules. Typically these will form a monolayer of lipid molecules. The molecules may thus form a sphere or particle. Typically, the hydrophobic tails of the lipid molecules will be oriented towards the inside of the micelle. Thus this can form a “non-aqueous cavity”, or a hydrophobic interior.

The micelles formed from the lipid according to the present invention may be formulated with one or more agents as described herein, thus forming a delivery vehicle. Such delivery vehicles comprising micelles may be prepared, for example, by dispersing an agent, such as siRNA, in water or aqueous buffer and combining with an appropriate aliquot of an aqueous solution of the lipid by rapid vortex mixing, preferably followed by sonication.

### Liposome

The lipid of the present invention may be used to form a liposome. Liposomes are typically spherical or particulate (closed) structures comprising one or more lipid bilayer membranes, and may contain an encapsulated aqueous volume, or a hydrophobic interior. Liposomes may contain many concentric lipid bilayers separated by an aqueous phase (multilamellar vesicles or MLVs), or alternatively, they may comprise a single membrane bilayer (unilamellar vesicles). The lipid bilayer is usually composed of two lipid monolayers having a hydrophobic “tail” region and a hydrophilic “head” region. In the membrane bilayer, the hydrophobic (nonpolar “tails” of the lipid monolayers orient toward the centre of the bilayer, whereas the hydrophilic (polar) “heads” orient toward the aqueous phase.

The liposomes described herein may include further lipids in addition to that of the present invention. The additional lipids may be for example neutral lipids or sterol lipids. Further lipid components that may be used in the liposomes are generally described in the literature. Generally, these are phospholipids—such as phosphatidylcholine, phosphatidylethanolamine, phosphatidylglycerol, phosphatidylserine, phosphatidic acid, phosphatidylinositol and/or sphingolipids. Additional components, for example, sterols—such as cholesterol—or other components—such as fatty acids (e.g., stearic acid, palmitic acid), dicetyl phosphate or cholesterol hemisuccinate, may be used. Moreover, the liposome membrane can also contain preservatives. The liposome membrane may also contain components, which modify their dispersion behaviour. They include, for example, PEGylated derivatives of phosphatidylethanolamine, lipids—such as GM 1—or conjugates of sugars and hydrophobic components—such as palmitic or stearic acid esters of dextran.

### Neutral Lipid

As used herein, the term “neutral lipid” refers to a lipid that is an uncharged or neutral zwitterionic form at a selected pH.

Neutral lipids suitable for inclusion in the liposome include, among many others: lecithins; phosphatidylethanolamines, such as DOPE (dioleoyl phosphatidylethanolamine), POPE (palmitoyloleoylphosphatidylethanolamine) and DSPE (distearoylphosphatidylethanol amine); phosphatidylcholine; phosphatidylcholines, such as DOPC (dioleoyl phosphatidylcholine), DPPC (dipalmitoylphosphatidylcholine) POPC (palmitoyloleoyl phosphatidylcholine) and DSPC (distearoylphosphatidylcholine); phosphatidylglycerol; phospha-tidylglycerols, such as DOPG (dioleoylphosphatidylglycerol), DPPG (dipalmitoylphosphatidylglycerol), and DSPG (distearoylphosphatidylglycerol); phosphatidylserines, such as dioleoyl- or dipalmitoylphospatidylserine; diphosphatidylglycerols; fatty acid esters; glycerol esters; diacylglycerols, sphingolipids; cardolipin; cephalin, cerebrosides; glycerol based lipids (e.g. cardiolipid); ceramides; and mixtures thereof.

Preferably, the neutral lipid is DOPE or DOPC.

### Sterol Lipid

A highly preferred sterol lipid group is cholesterol.

In one embodiment, the liposome comprises cholesterol and DOPE or DOPC, in addition to the lipid of the present invention.

The basic structure of liposomes may be made by a variety of techniques known in the art.

For example, liposomes have typically been prepared using a process whereby lipids suspended in organic solvent are evaporated under reduced pressure to a dry film in a reaction vessel. An appropriate amount of aqueous phase is then added to the vessel and the mixture agitated. The mixture is then allowed to stand, essentially undisturbed for a time sufficient for the multilamellar vesicles to form.

Liposomes may also be reproducibly prepared using a number of currently available techniques that are known in the art. The types of liposomes which may be produced using a number of these techniques include small unilamellar vesicles (SUVs), reverse-phase evaporation vesicles (REV) and stable plurilamellar vesicles (SPLV).

The liposomes formed from the lipid according to the present invention may be formulated with one or more agents as described herein, thus forming a delivery vehicle. For example, to combine one or more agents with the liposomes, a solution of the agent(s) in water or aqueous buffer may be added drop-wise to an aqueous solution of the liposomes under heavy vortexing. Preferably, in the case of siRNA, this is continued until a final siRNA concentration of about 0.1 mg/mL is reached. The addition step is preferably followed by sonication.

The liposomes or delivery vehicles described herein may be lyophilised or frozen.

It may also be desirable to include other ingredients in the liposome—such as diagnostic markers including radiolabels, dyes, chemiluminescent and fluorescent markers; contrasting media; imaging aids; agents and so forth.

In one aspect lipids suitable for use in imaging applications may incorporated in the liposome. The imaging lipid may a lipid selected from fluorescent lipids, magnetic resonance imaging lipids, nuclear magnetic resonance imaging lipids, electron microscopy and image processing lipids, electron spin resonance lipids and radioimaging lipids. Suitable and preferred lipids in each of these classes are given below.

### Fluorescent Lipids

Examples of fluorescent lipids are 1,2-Dioleoyl-sn-Glycero-3-Phosphoethanolamine-N-(5-dimethylamino-1-naphthalenesulfonyl, 1,2-Dioleoyl-sn-Glycero-3-Phosphoethanolamine-N-(1-pyrenesulfonyl),1,2-Dioleoyl-sn-Glycero-3-Phosphoethanolamine-N-(Carboxyfluorescein), 1-Oleoyl-2-[6-[(7-nitro-2-1,3-benzoxadiazol-4-yl)amino]hexanoyl]-sn-Glycero-3-Phospho-L-Serine, 25-{N-[(7-nitrobenz-2-oxa-1,3-diazol-4-yl)-methyl]amino}-27-norcholesterol, -Oleoyl-2-[6-[(7-nitro-2-1,3-benzoxadiazol-4-yl)amino]hexanoyl]-sn-Glycero-3-Phosphoethanolamine and 1,2-Dioleoyl-sn-Glycero-3-Phosphoethanolamine-N-(Lissamine Rhodamine B Sulfonyl).

### Lipids for Magnetic Resonance Imaging and Nuclear Magnetic Resonance Imaging

Examples of such lipids are Gd-DTPA-bis(stearylamide) (Gd-BSA); Gd-DTPA-bis(myrisitylamide) (GdDTPA-BMA); 1,2-Dimyristoyl-sn-Glycero-3-PhosphoEthanolamineDiethylene-TriaminePentaAcetate Gd3+ (DMPEDTPA:Gd3+); D35-1,2-Dihexanoyl-sn-Glycero-3-Phosphocholine; gadolinium (III) 2-{4,7-bis-carboxymethyl-10-[(N,N-distearylamidomethyl-N′-amido-methyl]-1,4,7,10-tetra-azacyclododec-1-yl}-acetic acid (Gd.DOTA.DSA); gadolinium (III) 1,4,7,10-tetraazacyclododecane-1,4,7,10-tetraacetic acid mono(N1-Cholesteryloxy-3-carbonyl-1,2-diaminoethane)amide (Gd.DOTA.Chol); gadolinium (III) 1,4,7,10-tetraazacyclododecane-1,4,7,10-tetraacetic acid mono(N1-distearoylphosphatidylethanolamine)amide (Gd.DOTA.DSPE) and gadolinium (III) Diethylene triamine 1,1,4,7,10-penta(acetic acid)-10-acetic acid mono(N1-Cholesteryloxy-3-carbonyl-1,2-diaminoethane)amide (Gd.DTPA.Chol).

### Electron Microscopy and Image Processing

An example of a suitable lipid is 1,2-Dioleoyl-sn-Glycero-3-{[N(5-Amino-1-Carboxypentyl)iminodiAcetic Acid]Succinyl}-(Nickel Salt).

### Electron Spin Resonance

An example of a suitable lipid is 1,2-Diacyl-sn-Glycero-3-Phosphotempocholine, 1-Palmitoyl-2-Stearoyl(n-DOXYL)-sn-Glycero-3-Phosphocholine.

### Radioimaging

An example of a suitable lipid is (99m)Tc-DTPA-bis(stearylamide); (99m)Tc-DTPA-bis(myrisitylamide).

### Amphiphilic Compounds

One or more amphiphilic compounds can optionally be incorporated into the liposomes in order to modify their surface properties. Amphiphilic compounds useful in this invention include, among many others; neoglycolipids—such as GLU4 and GLUT (see WO01/48233, FIG. 22), polyethyleneglycol lipids—such as N—(O-methoxy(polyoxyethylene) oxycarbonyl)-phosphatidylethanolamine, N-monomethoxy(polyoxyethylene) succinylphosphatidylethanol-amine and polyoxyethylene cholesteryl ether; nonionic detergents such as alkyl glycosides, alkyl methyl glucamides, sucrose esters, alkyl polyglycerol ethers, alkyl polyoxyethylene ethers and alkyl sorbitan oxyethylene ethers and steroidal oxyethylene ethers; block copolymers such as polyoxyethylene polyoxypropylene block copolymers

It may also be useful to incorporate aminoxy lipids (see, e.g. WO02/48170) coupling to PEG aldehydes (see, e.g. WO2006/016097) into the liposomes of the present invention.

### Delivery Vehicle

The lipids of the present invention may be used to form delivery vehicles for agents, in particular therapeutic agents. Such delivery vehicles may comprise (i) the lipid of the present invention and (ii) one or more agents.

The delivery vehicle may be based on liposomes comprising the lipid of the present invention and optional further lipids or additives as described above, which liposomes further comprise one or more agents. Such delivery vehicles may be made by contacting a liposome with one or more agents and any other components to be included in the liposome. Such agents may be included in the liposomes or by dissolving or dispersing the agent(s) in a suitable solvent and added to the liposome mixture prior to mixing.

Suitable delivery vehicles may also be described using the ABCD nanoparticle concept (Kostarelos, K. & Miller, A. D. Synthetic, self-assembly ABCD nanoparticles; a structural paradigm for viable synthetic non-viral vectors. Chem. Soc. Reviews 34, 970-994 (2005)). Such ABCD nanoparticles comprise one or more agents, such as a nucleic acid (A), condensed within functional concentric layers of chemical components designed for biological targeting (D), biological stability (C) and cellular entry/intracellular trafficking (B). Component B typically comprises lipids. Thus in the delivery vehicle of the present invention, component B comprises the lipid of the present invention, and optionally further comprises one or more polymers, or other lipids and additives as described above for the liposome. For example, component B may comprise DODAG/DOPE, DODAG/DOPC, DODAG/DOPC/Chol, etc.

The lipids of component B may typically comprise from 1 to 100 mol % of the lipid of the present invention. In one embodiment, the lipids of component B comprise from 10 to 50 mol % of the lipids of the present invention, preferably 15 to 30 mol %, such as about 20 mol %.

Components C and D are optional, such that the nanoparticles may comprise AB core nanoparticles (for example comprising one or more agents encapsulated by liposomes/micelles B in a non-covalent manner), ABC, ABD or ABCD particles.

Component C comprises a chemical component having stealth/biocompatibility properties, typically a polymer. As used herein, the term “polymer” refers to any polymer that comprises one or more functional groups that interact, bind or are coupled with one or more lipids contained as component B in a liposome.

The polymer C may be a naturally occurring polymer or a derivative thereof.

The polymer C may be a chemically modified polymer in which the polymer has been modified to include one or more functional groups.

In a preferred embodiment, one or more lipids B of the liposome are coupled to one or more polymers C. Advantageously, the lipid(s) that are coupled to the polymer(s) are exposed at the surface of the liposome such that the polymer remains at the liposome surface. Without being bound by any particular theory, it is believed that the polymer(s) will effectively coat the surface of the liposome through a plurality of interactions between the lipid(s) of the liposome and the polymer.

The coupling between the lipids and the polymers may be mediated by any type of interaction—such as hydrogen bonding interaction, a charge interaction, a hydrophobic interaction, a covalent interaction, a Van Der Waals interaction, or a dipole interaction. In a preferred embodiment, the interaction is mediated via a covalent interaction. Preferably, the covalent interaction occurs between one or more groups (e.g. functional groups) of the polymer and one or more lipids of the liposome.

One skilled in the art would be able to select suitable groups to achieve the desired interaction between one or more groups (e.g. functional groups) of the polymer and one or more lipids of the liposome.

In one preferred aspect, the covalent interaction occurs between one or more groups of the polymer and one or more functional groups of one or more lipids of the liposome selected from amine, thiol, alcohol, aminoxy, hydrazine, hydrazide, azides, isothiocyanate, aldehydes, ketones, isocyanates, maleimide, halides, tosylates, and esters. In one embodiment, aminoxy groups and hydrazine groups are preferred.

In one embodiment, the covalent interaction may occur between one or more aldehyde and/or ketone groups of the polymer and one or more aminoxy functional groups of one or more lipids of the liposome (e.g. present in addition to the lipid of the present invention).

Advantageously, the provision of a lipid comprising an aminoxy group allows for simple linking of polymers to the lipid via the aminoxy group. When reacted with a polymer comprising an aldehyde or ketone group, a compound is provided in which the polymer and lipid are linked via an amide group. Such a linkage may be simply prepared in a “one-pot” reaction. This methodology avoids extensive purification procedures by simple dialysis or excess, non-reacted reagents.

Preferably the polymer is selected from mono or bifunctional poly(ethylene glycol) (“PEG”), poly(vinyl alcohol) (“PVA”); other poly(alkylene oxides) such as poly(propylene glycol) (“PPG”); and poly(oxyethylated polyols) such as poly(oxyethylated glycerol), poly(oxyethylated sorbitol), and poly(oxyethylated glucose), and the like.

The polymers can be homopolymers or random or block copolymers and terpolymers based on the monomers of the above polymers, straight chain or branched, or substituted or unsubstituted similar to mPEG and other capped, monofunctional PEGs having a single active site available for attachment to a linker.

Specific examples of suitable polymers include poly(oxazoline), poly(acryloylmorpholine) (“PacM”), and poly(vinylpyrrolidone)(“PVP”). PVP and poly(oxazoline) are well known polymers in the art and their preparation and use in the syntheses described for mPEG should be readily apparent to the skilled artisan. PAcM and its synthesis and use are also described in the art.

In a preferred embodiment, the polymer is polyethylene glycol (PEG) with a functional aldehyde and/or ketone group or a chemical derivative thereof.

In a preferred embodiment, the polymer has a molecular weight of from 1000 to 5000, preferably about 2000. In a preferred embodiment, the polyethylene glycol (PEG) has a molecular weight of from 1000 to 5000, preferably about 2000.

In a preferred embodiment, the polymer has one or more functional groups capable of coupling to the one or more lipids. In a preferred embodiment, the polymer has one or two and only one or two functional groups capable of coupling to the one or more lipids.

Different sizes of PEG may be used, and may include mono- and bis-aldehyde PEG.

In one embodiment, the delivery vehicles of the present invention incorporate aminoxy lipids (see, e.g. WO02/48170) coupling to PEG aldehydes (see, e.g. WO2006/016097).

When present, component C, e.g. a polymer such as PEG, is typically incorporated in an amount of 0.1 to 15 mol % of the combined lipid components (B). Examples of ABC formulations include DODAG/DOPE/Chol-PEG⁵⁰⁰⁰ and DODAG/DOPC/Chol/DOPE-PEG²⁰⁰⁰.

Component D comprises a chemical component designed for biological targeting. Targeted delivery may therefore be achieved by the addition of one or more components D (e.g. targeting moieties)—such as peptides, proteins, carbohydrates, antibodies and/or other ligands—to the liposome, preferably the surface of the liposome. Advantageously, the component(s) D are coupled to the surface of the liposome via an interaction between the component(s) D and one or more lipids B of the liposome that are exposed at the liposome surface.

Advantageously, this may enable delivery of the agent A (e.g. siRNA) to specific cells, organs and tissue that can bind the component(s) D (such as the targeting moiety). Typically, the binding between the cells, organs and tissues will be via a specific binding between cells, organs and/or tissues and the component(s) D.

In one embodiment, the cells, organs and tissues may be or may be derived from liver. As used herein, the term “liver cell” refers to a cell that is located in the liver. Liver cells may include but are not limited to cancerous liver cells, hepatocytes, Kupffer cells, Ito cells endothelial cells lining the hepatic sinusoids, vascular endothelial cells lining the hepatic blood vessels, and any cells of any origin which happen to reside in the liver (e.g., metastic cancer cells of ectopic origin). Preferably, the liver cell is hepatocyte (e.g. HepG2 cells). Thus, the non-viral delivery vehicles described herein may exhibit a strong accumulation in such cells, organs and tissues.

In another embodiment, the cells, organs and tissues may be or may be derived from the spleen, lung and/or lymph nodes.

The interaction between one or more cells, organs and/or tissues and component(s) D is typically dependent upon the presence of a particular structural feature of the cells, organs and/or tissues—such as an antigeneic determinant or epitope—that is recognised by the component(s) D, thereby allowing an interaction (eg. binding) to occur.

The component(s) D may be selected from the group consisting of a carbohydrate and/or another ligand.

Preferably, the carbohydrate is a sugar selected from the group consisting of glucose, mannose, lactose, fructose, maltotriose, maltoheptose.

Many different ligands may be employed, depending upon the site targeted for liposome delivery.

The ligand may be designed or obtained from a library of compounds which may comprise peptides, as well as other compounds such a small organic molecules.

By way of example, the ligand may be a natural substance, a biological macromolecule, or an extract made from biological materials such as bacteria, fungi, or animal (particularly mammalian) cells or tissues, an organic or an inorganic molecule, a synthetic ligand, a semi-synthetic ligand, a structural or functional mimetic, a peptide, a peptidomimetic, a derivatised ligand, a peptide cleaved from a whole protein, or a peptide synthesised synthetically (such as, by way of example, either using peptide synthesiser or by recombinant techniques or combinations thereof, a recombinant ligand, an antibody, a natural or a non-natural ligand, a fusion protein or equivalent thereof and mutants, derivatives or combinations thereof).

Preferably, the ligand is an antibody.

As used herein, the term “antibody” refers to complete antibodies, bi-specific antibodies or antibody fragments, and includes Fv, ScFv, Fab′ and F(ab′)₂, monoclonal and polyclonal antibodies, engineered antibodies including chimeric, CDR-grafted and humanised antibodies, and artificially selected antibodies produced using phage display or alternative techniques.

A chimeric antibody refers to a genetically engineered fusion of parts of a mouse antibody with parts of a human antibody. Generally, chimeric antibodies contain approximately 33% mouse protein and 67% human protein.

Humanised antibodies may be obtained by replacing the constant region of a mouse antibody with human protein, but by also replacing portions of the antibody's variable region with human protein. Generally, humanised antibodies are 5-10% mouse and 90-95% human.

A more sophisticated approach to humanised antibodies involves not only providing human-derived constant regions, but also modifying the variable regions as well. This allows the antibodies to be reshaped as closely as possible to the human form.

Preparation of antibodies is performed using standard laboratory techniques antibodies may be obtained from animal serum, or, in the case of monoclonal antibodies or fragments thereof, produced in cell culture. Recombinant DNA technology may be used to produce the antibodies according to established procedure, in bacterial or preferably mammalian cell culture. The selected cell culture system preferably secretes the antibody product.

Antibodies may be selected and generated using phage display technology. Methods for the construction of bacteriophage antibody display libraries and lambda phage expression libraries are well known in the art.

Advantageously, when the ligand is an antibody, the lipoplex coupled antibody demonstrates substantially all of its activity.

Preferably, the ligand is targeted to a receptor. Such a ligand could be RGD peptide (integrin receptor), folate (folate receptor) and/or transferrin (transferrin receptor).

When present, the amount of component D will depend on the desired application. Typically, component D is incorporated in an amount of 0.05 to 10 mol % of the combined lipid components (B).

In one embodiment, the delivery vehicle comprises AB nanoparticles. These may consist of one or more agents A and one more lipids according to the present invention as component B.

The nanoparticles may be made by a “pre-modification” method, wherein a liposome comprising the desired B and optional C components is formulated prior to addition of the one or more agents. ABC nanoparticles comprising PEG may also be formulated using a “post-modification” method, which involves first forming AB core nanoparticles and then mixing with PEG-lipid in the form of micelles. Alternatively, the C component, such as PEG-polymer, may be equipped with reactive functional groups that bioconjugate in aqueous conditions with complementary functional groups presented on the outside surface of the AB nanoparticle. Such a “post-coupling” process may extend by analogy to formulation of ABD or ABCD nanoparticles.

Typically, hydrophilic active agents are encapsulated into liposomes or delivery vehicles by hydrating the dry lipid film with an aqueous solution of active agent. In this manner, the agent is passively encapsulated in the interlamellar spaces of the liposome or delivery vehicle. Alternatively, hydrophilic, water-soluble active agents may be encapsulated in liposomes or delivery vehicles by a reverse loading technique. This method involves the dispersal of neutrally charged drugs or other active agents in the aqueous phase of a liposome or delivery vehicle preparation, which allows the uncharged drugs or other active agents to permeate into liposomes or delivery vehicles via the lipid bilayer. The pH of the liposome or delivery vehicle solution is adjusted to create a charge on the active agent, rendering the active agent unable to pass back through the bilayer and into the external medium, thereby entrapping the active agent in the liposome or delivery vehicle. Lipophilic active agents (e.g., hydrophobic drugs or other active agents or water-insoluble drugs or other active agents) may be incorporated into liposomes or delivery vehicles by partitioning. In this respect, the agent is dissolved along with the lipophilic ingredients in a suitable non-polar solvent. The resulting solution can either be dried and mixed with a polar solvent as described above, or directly added to the aqueous phase and extracted. In this manner, the agent is incorporated into the lipid portion of the liposome or delivery vehicle bilayer. The agent may be dissolved in a third solvent or solvent mix and added to the mixture of polar solvent with the lipid film prior to homogenising the mixture.

The lipids may be part of a pre-formed liposome or delivery vehicle comprising one or more, preferably, two or more lipid constituents. The final complex may be stored at approximately −20° C.

Surprisingly, it has been found that freezing the liposomes or delivery vehicles prior to use can enhance the quality (e.g. enhance or improve the bio-distribution) of the liposomes or delivery vehicles described herein.

Accordingly, a process for enhancing or improving the bio-distribution of a liposome or delivery vehicle may comprise the steps of: (a) providing a liposome or delivery vehicle as described herein; (b) freezing the liposome or delivery vehicle; and (c) thawing the liposome or delivery vehicle prior to use.

In a further aspect, a process for enhancing the efficiency of delivery vehicle-mediated delivery may comprise the steps of: (a) providing a delivery vehicle as described herein; (b) freezing the delivery vehicle; and (c) thawing the delivery vehicle prior to use.

Preferably, the liposome or delivery vehicle is frozen at a temperature of at least about −16° C., more preferably at least about −17° C., more preferably, at least about −18° C., more preferably, at least about −19° C. and most preferably, at least about −20° C.—such as from about −20° C. to about −25° C.

Typically, the liposome or delivery vehicle is frozen using liquid nitrogen or carbo-ice and is brought below −20° C. The sample is then kept at about −20° C. before re-using. The liposome or delivery vehicle can be kept at about −20° C. for an extended period of time prior to usage. Suitably, the liposome or delivery vehicle is stable at room temperature or at about 4° C. for at least one month.

It is possible to combine the components of the delivery vehicle in any order. Where further components are to be added, they may be added at any stage, for example together with the one or more agents.

Advantageously, the delivery vehicles described herein have a high loading capacity for the one or more agents. By way of example, the final siRNA concentration may be up to about 0.5 mg/ml or more (e.g. about 0.25 mg/ml or about 0.1 mg/ml, for example from 0.05-0.3 mg/ml).

Although the delivery vehicles may be particularly well suited for pharmaceutical use, they are not limited to that application, and may be designed for food use, agricultural use, for imaging applications, and so forth as described herein.

Preferably, the lipid:agent ratio is about 1-30:1 (w/w), for example 1-15:1 (w/w), such as 1-10:1 (w/w) or 1-5:1 (w/w).

Preferably, the lipid:siRNA ratio is about 1-30:1 (w/w), preferably 1-15:1 (w/w), preferably 1-10:1 (w/w), more preferably 1-5:1 (w/w).

### Agent

The micelles or liposomes described herein may be formulated with one or more agents in order to prepare a delivery vehicle that is suitable for the delivery of one or more agents in vivo or in vitro. Delivery vehicles comprising the lipid of the present invention and one or more agents are also described herein.

The agent may typically be present in the interior of the micelle/liposome/delivery vehicle structure, or it may be incorporated with the lipid, e.g. in the lipid bilayer.

The agent may be an agent that can be entrapped in lipid vesicles, including water-soluble agents that can be stably encapsulated in the aqueous compartment of the vesicles, lipophilic compounds that stably partition in the lipid phase of the vesicles, or agents that can be stably attached, e.g., by electrostatic attachment to the outer vesicle surfaces. Exemplary water-soluble compounds include small, water-soluble organic compounds, peptides, proteins, nucleic acid (eg, DNA, RNA, mRNA, siRNA, antisense oligonucleotides). These compounds may be natural or synthetic.

The agent may be an organic compound or other chemical. The agent may be a compound, which is obtainable from or produced by any suitable source, whether natural or artificial. The agent may be an antibody, for example, a polyclonal antibody, a monoclonal antibody or a monoclonal humanised antibody.

The agent may be a drug, compound or analogue thereof, a bioactive agent or a biochemical. For example, the agent may be a known drug or compound or an analogue thereof.

The agent may be designed or obtained from a library of compounds, which may comprise peptides, as well as other compounds, such as small organic molecules.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a peptide cleaved from a whole protein, or a peptide synthesised synthetically (such as, by way of example, either using a peptide synthesiser or by recombinant techniques or combinations thereof), a recombinant agent, an antibody, a natural or a non-natural agent, a fusion protein or equivalent thereof and mutants, derivatives or combinations thereof.

The agent may be an organic compound. For some instances, the organic compound will comprise two or more hydrocarbyl groups.

The agent may contain halo groups—such as fluoro, chloro, bromo or iodo groups.

The agent may contain one or more of alkyl, alkoxy, alkenyl, alkylene and alkenylene groups—which may be unbranched- or branched-chain.

The agent may exist as stereoisomers and/or geometric isomers—eg. the agent may possess one or more asymmetric and/or geometric centres and so may exist in two or more stereoisomeric and/or geometric forms. The present invention contemplates the use of all the individual stereoisomers and geometric isomers of those agents, and mixtures thereof.

The agent may be any chemical or substance that is desired to be applied, administered or used in a liposome, and may include, but is not limited to pesticides, herbicides, cosmetic agents and perfumes, food supplements including vitamins and minerals, flavourings, and other food additives, imaging agents, dyes, fluorescent markers, radiolabels, plasmids, vectors, viral particles, toxins, catalysts, and so forth.

The agent may include one or more biologically active agents and includes any molecule that acts as a beneficial or therapeutic compound, when administered to an animal, preferably a mammal, more preferably a human, in order to prevent, alleviate or treat a disease. This may include: preventing the disease from occurring in a subject which may be predisposed to the disease but has not yet been diagnosed as having it; inhibiting the disease, i.e. arresting its development; or relieving the disease, i.e. causing regression of the disease.

Examples of agents include, but are not limited to, anti-inflammatory agents; anti-cancer and anti-tumour agents; anti-microbial and anti-viral agents, including antibiotics; anti-parasitic agents; vasodilators; bronchodilators, anti-allergic and anti-asthmatic agents; peptides, proteins, glycoproteins, and lipoproteins; carbohydrates; receptors; growth factors; hormones and steroids; neurotransmitters; analgesics and anaesthetics; narcotics; catalysts and enzymes; vaccines or genetic material.

Suitable drugs include, but are not limited to hydrophilic drugs, hydrophobic drugs, and water-insoluble drugs. A hydrophilic drug or other active agent is readily dissolved in water. A hydrophobic drug or other active agent has a low affinity for water, and does not readily dissolve in aqueous solutions. The dissolution of hydrophobic drugs or other active agents in water, however, is not impossible, and can be achieved under certain conditions that are known to those skilled in the art. Hydrophobic drugs or other active agents typically are dissolved in non-polar (e.g., lipophilic) solvents. In contrast, a water-insoluble drugs or other active agent cannot dissolve in water under any circumstances. In this case, organic solvents can be used to dissolve water-insoluble drugs or other active agents. Hydrophilic active agents may be included in the interior of the liposomes such that the liposome bilayer creates a diffusion barrier preventing it from randomly diffusing throughout the body.

Preferably, the drug is a hydrophobic or water-insoluble drug. Advantageously, such drugs are well solubilised in the lipid bilayer of liposomes.

In one preferred embodiment, the agent comprises a nucleic acid or a polynucleotide (which may be single or double-stranded), for example DNA, RNA, mRNA, siRNA or antisense olignucleotides. These may be naturally occurring or synthetic.

In one embodiment, the liposome/delivery vehicle preferably comprises two or more agents (e.g., drugs or other active agents). The two or more agents may be any combination of one or more hydrophobic agent(s), one or more water-insoluble agent(s), and/or one or more hydrophilic agents. Each of the hydrophilic agent(s) is present in the aqueous cavity of the liposome/delivery vehicle, whereas each of the hydrophobic agent(s) and/or water-insoluble agent(s) is present in the lipid bilayer of the liposome/delivery vehicle.

In a preferred embodiment, the liposome/delivery vehicle comprises at least one hydrophilic agent and at least one water-insoluble agent. Alternatively, the liposome/delivery vehicle can comprise at least one hydrophilic agent and one hydrophobic agent. Most preferably, the liposome/delivery vehicle comprises one hydrophilic agent in combination with one water-insoluble agent or one hydrophobic agent. Thus, in such liposome/delivery vehicle compositions, the water-soluble agent is present in the aqueous cavity of the liposome/delivery vehicle, while the water-insoluble agent or the hydrophobic agent is present in the lipid bilayer of the liposome/delivery vehicle.

In another embodiment, the liposome/delivery vehicle comprises two or more agents, each of which is hydrophilic. Each of the two or more agents is present in the aqueous cavity of the liposome/delivery vehicle, while no agents are present in the lipid bilayer of the liposome/delivery vehicle.

In another embodiment, the liposome/delivery vehicle comprises two or more water-insoluble or hydrophobic agents. In such liposome/delivery vehicle compositions, each of the two or more agents is present in the lipid bilayer of the liposome/delivery vehicle, while no agents are present in the aqueous cavity of the liposome/delivery vehicle.

The combination of the two or more agents may include drugs, nutritional supplements, vitamins, hormones, minerals, enzymes, proteins, and/or peptides. One or more of the agents in the composition can be selected from this group. In one embodiment, the combination of agents—such as the two or more drugs or other active agents—may be cytotoxic to a particular cell or cell type, and preferably the combination is cytotoxic to cancer (e.g. tumour) cells. Thus, the combination of the two or more agents can include two or more drugs or other agents that are cytotoxic to cancer cells.

In one embodiment, the drugs or other active agents are preferably anticancer agents—such as chemotherapeutic agents—in that they are capable of inducing (either directly or indirectly) cancer cell or tumour cell cytotoxicity. Examples of such anticancer agents include, but are not limited to, mitoxantrone (as described in WO02/32400), taxanes (as described in WO01/70220 and WO00/01366), paclitaxel, camptothecin, camptothecin derivaties (as described in WO02/058622 and WO04/017940), topotecan, gemcitabine (as described in WO04/017944), vinorelbine (as described in WO03/018018), vinblastine, anthracyclines, adria, adriamycin, adriamycine, capecitabine, doctaxel, doxorubicin; didanosine (ddl), stavudine (d4T), antisense oligonucleotides—such as c-raf antisense oligonucleotide (RafAON) (as described in U.S. Pat. No. 6,126,965 and U.S. Pat. No. 6,559,129), antibodies—such as herceptin, immunotoxins, hydroxyurea, melphalan, chlormethine, extramustinephosphate, uramustine, ifosfamide, mannomustine, trifosfamide, streptozotocin, mitobronitol, mitoxantrone, methotrexate, 5-fluorouracil, cytarabine, tegafur, idoxide, taxol, daunomycin, daunorubicin, bleomycin, amphotericin (e.g., amphotericin B), carboplatin, cisplatin, BCNU, vincristine, camptothecin, mitomycin, doxorubicin, etopside, histermine dihydrochloride, tamoxifen, cytoxan, leucovorin, oxaliplatin, irinotecan (as described in WO03/030864), 5-irinotecan, raltitrexed, epirubicin, anastrozole, proleukin, sulindac, EKI-569, erthroxylaceae, cerubidine, docetaxel, cytokines—such as interleukins (e.g. interleukin-2), ribozymes, interferons, oligonucleotides, and functional derivatives of the foregoing.

In a preferred embodiment, at least one of the two or more agents present in the liposome/delivery vehicle is a nucleic acid, preferably siRNA.

In another preferred embodiment, at least one of the two or more agents present in the liposome/delivery vehicle is a hydrophobic drug, preferably taxol.

In another preferred embodiment, at least two of the two or more agents present in the liposome/delivery vehicle are siRNA and taxol.

Other suitable agents may be selected from, for example, proteins, enzymes, hormones, nucleotides, polynucleotides, nucleoproteins, polysaccharides, glycoproteins, lipoproteins, polypeptides, steroids, terpenoids, retinoids, anti-ulcer H2 receptor antagonists, antiulcer drugs, hypocalcemic agents, moisturizers, cosmetics, etc. Active agents can be analgesics; anesthetics; anti-arrythmic agents, antibiotics; antiallergic agents, antifungal agents, antihypertensive agents (e.g. dihydropyridines, antidepressants, cox-2 inhibitors); anticoagulants; antidepressants; antidiabetic agents, anti-epilepsy agents, antiinflammatory corticosteroids.

The agents or drugs can be nephrotoxic, such as cyclosporins and amphotericin B, or cardiotoxic, such as amphotericin B and paclitaxel. Additional examples of drugs which may be delivered include but are not limited to, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isofluorophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17a-hydroxyprogesterone acetate, 19-norprogesterone, norgestrel, norethindrone, norethisterone, norethiederone, progesterone, norgesterone, norethynodrel, aspirin, indomethacin, naproxen, fenoprofen, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, haloperidol, zomepirac, ferrous lactate, vincamine, diazepam, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat captopril, ramipril, famotidine, nizatidine, sucralfate, etintidine, tetratolol, minoxidil, chlordiazepoxide, diazepam, amitriptyline, and imipramine. Further examples are proteins and peptides which include, but are not limited to, bone morphogenic proteins, insulin, heparin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, somatotropins (e.g., bovine somatotropin, porcine somatotropin, etc.), oxytocin, vasopressin, GRF, somatostatin, lypressin, pancreozymin, luteinizing hormone, LHRH, LHRH agonists and antagonists, leuprolide, interferons, interleukins, growth hormones (e.g. human growth hormone and its derivatives such as methione-human growth hormone and des-phenylalanine human growth hormone, bovine growth hormone, porcine growth hormone, insulin-like growth hormone, etc.), fertility inhibitors such as the prostaglandins, fertility promoters, growth factors such as insulin-like growth factor, coagulation factors, pancreas hormone releasing factor, analogues and derivatives of these compounds, and pharmaceutically acceptable salts of these compounds, or their analogues or derivatives.

The term “derivative” or “derivatised” as used herein includes chemical modification of an agent. Illustrative of such chemical modifications would be replacement of hydrogen by a halo group, an alkyl group, an acyl group or an amino group.

The agent may be a modified agent—such as, but not limited to, a chemically modified agent.

The chemical modification of an agent may either enhance or reduce hydrogen bonding interaction, charge interaction, hydrophobic interaction, Van Der Waals interaction or dipole interaction.

The present invention also encompasses the use of variants, homologues, derivatives and fragments thereof of the nucleotide sequences described herein. Here, the term “homologue” means an entity having a certain homology with the subject nucleotide sequences. Here, the term “homology” can be equated with “identity”. These terms are well understood by those skilled in the art and are described, for example in WO 2006/016097.

siRNA

In one preferred embodiment, the agent is siRNA.

The siRNA agent may comprise partially purified RNA, substantially pure RNA, synthetic RNA, or recombinantly produced RNA, as well as altered RNA that differs from naturally-occurring RNA by the addition, deletion, substitution and/or modification of one or more nucleotides.

Such alterations can include the addition of non-nucleotide material—such as modified nucleotides—to, for example, the end(s) of the siRNA or to one or more internal nucleotides of the siRNA, including modifications that make the siRNA resistant or even more resistant to nuclease digestion.

A number of different types of modifications are known in the art. These include methylphosphonate and phosphorothioate backbones and/or the addition of acridine or polylysine chains at the 3′ and/or 5′ ends of the molecule. The nucleotide sequences may be modified by any method available in the art. Such modifications may be carried out to enhance the in vivo activity or life span of the siRNA.

One or both strands of the siRNA may comprise a 3′ overhang.

Thus, the siRNA may comprise at least one 3′ overhang of, for example, from 1 to about 6 nucleotides (which includes ribonucleotides or deoxynucleotides) in length. If both strands of the siRNA molecule comprise a 3′ overhang, the length of the overhangs can be the same or different for each strand.

In order to enhance the stability of the siRNA, the 3′ overhangs may be stabilised against degradation. The overhangs may be stabilised by including purine nucleotides—such as adenosine or guanosine nucleotides. Alternatively, substitution of pyrimidine nucleotides by modified analogues may be tolerated and may not affect the efficiency of RNAi degradation.

Typically, the siRNA will be in the form of isolated siRNA comprising short double-stranded RNA from about 17 nucleotides to about 29 nucleotides in length—such as approximately 19-25 contiguous nucleotides in length—that are targeted to a target mRNA. The siRNA comprise a sense RNA strand and a complementary antisense RNA strand annealed together by standard Watson-Crick base-pairing interactions. The sense strand comprises a nucleic acid sequence which is identical to a target sequence contained within the target mRNA.

As used herein, the term “isolated siRNA” means that the siRNA is altered or removed from the natural state through human intervention. An isolated siRNA can exist in substantially purified form, or can exist in a non-native environment such as, for example, a cell into which the siRNA has been delivered.

The sense and antisense strands of the siRNA can comprise two complementary, single-stranded RNA molecules or can comprise a single molecule in which two complementary portions are base-paired and are covalently linked by a single-stranded hairpin.

It is understood that human mRNA may contain target sequences in common with their respective alternative splice forms, cognates or mutants. A single siRNA comprising such a common targeting sequence can therefore induce RNAi-mediated degradation of different RNA types which contain a common targeting sequence. Techniques for designing siRNA are known in the art and are described, for example, in WO 2006/016097. Further methods for the design of siRNA may be found at the websites of, for example, QIAGEN, Ambion and Ocimum Biosolutions.

Although siRNA silencing is highly effective by selecting a single target in the mRNA, it may be desirable to design and employ two or more independent siRNA duplexes to control the specificity of the silencing effect.

siRNA may be obtained using a number of techniques known to those of skill in the art. For example, the siRNA may be chemically synthesized using appropriately protected ribonucleoside phosphoramidites and a conventional DNA/RNA synthesiser. The siRNA may be synthesized as two separate, complementary RNA molecules, or as a single RNA molecule with two complementary regions.

siRNA may also be purchased from several companies—such as Dharmacon (USA), Qiagen GmbH (Hilden, Germany) and Sigma (USA).

The siRNA may be labelled. By way of example, the siRNA may be labelled with a 3′-FITC label anti-GFP.

siRNA may be recombinantly produced using methods known in the art. For example, siRNA may be expressed from recombinant circular or linear DNA plasmids using any suitable promoter. The recombinant plasmids of the invention can also comprise inducible or regulatable promoters for expression of the siRNA in a particular tissue or in a particular environment (e.g. particular intracellular environment)—such as the blood or blood stream.

The siRNA expressed from recombinant plasmids can either be isolated from cultured cell expression systems by standard techniques. siRNA may be expressed from a recombinant plasmid either as two separate, complementary RNA molecules, or as a single RNA molecule with two complementary regions.

siRNA sequences may include those that are of therapeutic and/or diagnostic application—such as sequences encoding cytokines, chemokines, hormones, antibodies, engineered immunoglobulin-like molecules, a single chain antibody, fusion proteins, enzymes, immune co-stimulatory molecules, immunomodulatory molecules, a transdominant negative mutant of a target protein, a toxin, a conditional toxin, an antigen, a tumour suppressor protein, a growth factor, a membrane protein, a vasoactive proteins and peptides, an anti-viral protein and/or a ribozyme.

The target mRNA may be or may be derived from the anti-apoptotic protein livin-2 (U73857), which is used for stimulating caspase-3, resulting in the onset of apoptosis in the cell line transfected with siRNA. One example of such a siRNA sequence which targets this mRNA is 5′-GGG CGU GGU GGG UUC UUG AGC-3′(SEQ ID NO: 15).

The target mRNA may be or may be derived from HBV, HCV and/or P-pg.

The siRNA may be targeted to a target mRNA that is or is derived from HBV, HCV and/or P-glycoprotein.

In particular, the sequences of Hepatitis B virus include Hepatitis B virus isolate 2-AII-BR large S protein (S) gene (Accession number AY344099.1); Hepatitis B virus isolate 6-AIII-BR large S protein (S) gene (Accession number AY344104.1); Hepatitis B virus isolate j13 small surface protein (S) gene (Accession number AY639927.1); Hepatitis B virus isolate j7 small surface protein (S) gene (Accession number AY639924.1); Hepatitis B virus isolate 17993 (Accession number AY217367.1); and/or Hepatitis B virus isolate Q7-1 (Accession number AY217365.1).

Preferably, the HBV siRNA sequences are directed against the conserved sequence of the HBV core gene. More preferably, the HBV siRNA sequences are selected from the group consisting of: 5′-GCGGGACGUCCUUUGUUUACG (SEQ ID NO: 1) (siRNA 1407) and 5′-GGUCUGCGCACCAUCAUCAUG (SEQ ID NO: 11) (siRNA 1794). These sequences were obtained using siDIRECT directed against a conserved sequence of the HBV genome (RNAi Co). All of these 21 nt RNA sequences with their appropriate complementary sequences have been chemically synthesized by Dharmacon (Colorado, USA). All sequences were PAGE purified. Both sequences revealed a potent pattern of downregulation of the HBV surface antigen.

The HCV virus is a positive stranded RNA virus containing a single, long open reading frame that encodes structural and non-structural proteins. Translation of the viral genome is mediated by an internal ribosomal entry site (IRES) which is located in the untranslated region at the 5′ terminus (5′-UTR; Accession Number D31603), which is also conserved in 99.6% of all virus strains. Therefore, it constitutes an ideal target for an siRNA drug. Similarly, the 3′-UTR (accession No D63922) is highly conserved and has been demonstrated to exhibit an important role for the virus replication in vivo.

The sequences of Hepatitis C virus include human hepatitis virus C capsid and envelope:x proteins (Accession number M55970.1); the 5′-UTR region (341 nt) that is conserved throughout all HCV isolates (Accession number M55970.1); and/or non structural proteins—such as NS3, NS4, NS5A and NS5B.

The sequences of Hepatitis C virus include modified forms of hepatitis C virus NS3 protease (Accession number BD270935.1).

Preferably, the one or more siRNA sequences are directed towards the untranslated region at the 5′ terminus (5′-UTR; Accession Number D31603) or the 3′-UTR (Accession No D63922) of HCV. More preferably, the HCV sequences are selected from the group consisting of:

HCVIA146:

| | |
|---|---|
| 5′-GUCACGGCUAGCUGUGAAAdTdT;; | (SEQ ID NO: 16) |
| HCVIA185: | |
| 5′-UGCAGAGAGUGCUGAUAUTdTdT;; | (SEQ ID NO: 17) |
| HCVIA205: | |
| 5′ UGGCCUCUCUGCAGAUCAUdTdT;; | (SEQ ID NO: 18) |
| HCVIA56-5′-UTR: | |
| 5′ UACUGUCUUCACGCAGAAAdTdT;; | (SEQ ID NO: 19) |
| HCVIA210-5′-UTR | |
| 5′ CGCUCAAUGCCUGGAGAUUdTdT;; | (SEQ ID NO: 20) |
| HCVIA211-5′-UTR | |
| 5′ GCUCAAUGCCUGGAGAUUUdTdT;; | (SEQ ID NO: 21) |
| and | |
| HCVIA258-5′-UTR: | |
| 5′-GUAGUGUUGGGUCGCGAAAdTdT. | (SEQ ID NO: 22) |

All of the these 19 nt RNA sequences have been chemically synthesised by Dharmacon (Colorado, USA) with two DNA base pairs dTdT overhangs at both 3′ strands. All sequences were PAGE purified. The efficacies of the individual sequences were compared to siRNA331 (5′-GGUCUCGUAGACCGUGCAC; SEQ ID NO:23) described by Yokota et al, (2003) EMBO Reports 4(6),602ff.

The sequences of P-glycoprotein (MDR1 gene product) includes the Homo sapiens P-glycoprotein (ABCB1) (Accession number AF399931.1).

Typically, the siRNA will be termed “therapeutic siRNA”. As used herein, the term “therapeutic siRNA” refers to any siRNA that has a beneficial effect on the subject. Thus, “therapeutic siRNA” embraces both therapeutic and prophylactic siRNA.

The agent and/or the siRNA may be prepared by chemical synthesis techniques, as described for example in WO 2006/016097.

### Therapeutic Uses

The lipid of the present invention, or the micelles, liposomes or delivery vehicles formed therefrom, may be used in therapy or diagnosis. Thus, the present invention provides a lipid, micelle, liposome or delivery vehicle as described herein for use in therapy or diagnosis. The present invention further provides a lipid, micelle, liposome or delivery vehicle as described herein for use in treatment of a disorder or condition or disease, such as a genetic disorder or condition or disease, for delivery of one or more agents or for imaging. The present invention also provides use of a lipid, micelle, liposome or delivery vehicle as described herein in the manufacture of a medicament for treatment of a (genetic) disorder or condition or disease, for delivery of one or more agents or for imaging.

The present invention also provides a method for delivery of one or more agents to cells, for example for improving the efficiency of delivery, said method comprising: prior to administering the one or more agents to a patient, formulating said agent(s) with an effective amount of a delivery vehicle comprising a lipid as described herein. Preferably, the agent comprises a therapeutic agent.

The present invention also provides a method for delivering one or more agents to one or more cells comprising administering a composition comprising a) a lipid as described herein and b) one or more agents. Preferably, the agent comprises a therapeutic agent.

The present invention also provides a method for treatment of a disorder or condition or disease, such as a genetic disorder or condition or disease, in a patient in need thereof, comprising administering a pharmaceutical composition comprising a) a lipid as described herein and b) one or more agents. Preferably, the agent comprises a therapeutic agent.

### Diseases

Aspects of the present invention may be used for the treatment and/or prevention of diseases such as those listed in WO-A-98/09985.

For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; diseases associated with viruses and/or other intracellular pathogens; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue. Specific cancer related disorders include but not limited to: solid tumours; blood born tumours such as leukemias; tumor metastasis; benign tumours, for example hemangiomas, acoustic neuromas, neurofibromas, trachomas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; wound granulation; corornay collaterals; cerebral collaterals; arteriovenous malformations; ischeniic limb angiogenesis; neovascular glaucoma; retrolental fibroplasia; diabetic neovascularization; heliobacter related diseases, fractures, vasculogenesis, hematopoiesis, ovulation, menstruation and placentation.

Aspects of the present invention may be used for the treatment and/or prevention of diabetes—such as diabetes I and II.

Aspects of the present invention may also be used for the treatment and/or prevention of cancer.

Aspects of the present invention may also be used for the treatment and/or prevention of a disease, disorder or condition that is or is associated with liver disease and/or liver damage.

Liver damage may be associated with exposure to alcohol, hepatotoxic drugs and combinations thereof. For example, damaging agents may include anti-convulsants, phenyloin, carbamazepine and phenobarbital, recreations drugs—such as ecstasy (3,4-methylenedioxymethamphetamine), antituberculosis agents and chemotherapeutic agents—such as isoniazid and rifampicin.

Liver damage may also be associated with infectious agents—such as bacterial, parasitic, fungal and viral infections. For example, liver damage may result from Aspergillus fungal infections, Schistosoma parasitic infections and a variety of viral infections—such as adenovirus, retrovirus, adeno-associated virus (AAV), hepatitis virus A, hepatitis virus B, hepatitis virus C, hepatitis virus E, herpes simplex virus (HSV), Epstein-Barr virus (EBV) and paramyxovirus infections.

Liver diseases may include, but are not limited to, acute hepatitis, fulminant hepatitis, chronic hepatitis, hepatic cirrhosis, fatty liver, alcoholic hepatopathy, drug induced hepatopathy (drug addiction hepatitis), congestive hepatitis, autoimmune hepatitis, primary biliary cirrhosis, hepatic porphyria, pericholangitis, sclerosing cholangitis, hepatic fibrosis and chronic active hepatitis.

Preferably, the disease is a disease that can be treated via circulation of the delivery vehicles in the blood stream.

Preferably, the disease is a disease that can be treated via parenteral administration.

In another embodiment, the disease is an inflammatory disease—such as inflammatory bowel disease (eg. Crohn's disease, ulcerative colitis, IBS) or gastritis—or chromic inflammation (eg. Rheumatoid Arthritis—such as Rheumatoid Arthritis).

In another embodiment, the disease is a brain-related disease—such as Alzheimer's disease, Parkinson's disease, Huntington's disease, Amyotrophic Lateral Sclerosis and Multiple sclerosis.

### Cancer

In one preferred embodiment, the disease to be treated is cancer, since delivery vehicle formulations described herein accumulate in vivo in subcutaneous tumour xenografts. The cancer may be selected from the group consisting of acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia, neuroblastoma and Wilms' tumor.

Preferably, the delivery vehicles described herein accumulate in tumours at about 1, 2, 3, 4, 5, 6, 7, 8, 9 or 10% or more of the total injected dose/gram tumour. More preferably, the delivery vehicles described herein accumulate in tumours at about 1-5% or more, more preferably, 2-5% or more, more preferably, 3-5% or more preferably, 4-5% or more, most preferably, 4.5% or more of the total injected dose/gram tumour.

Preferably, the delivery vehicles accumulate in tumours after 1 hour and remain there for at least 2, 3, 4, 5, 6, 7, 8, 9, 10, 11, 12, 13, 14, 15, 16, 17, 18, 19, 20, 21, 22, 23, 24 or 25 hours. More preferably, the delivery vehicles accumulate in tumours after 1 hour and remain there for at least 2-20 hours, more preferably, at least 5-20 hours, more preferably, at least 10-20 hours, more preferably, at least 15-20 hours, more preferably, at least 17-20 hours, more preferably at least 18-20 hours, more preferably, at least 19-20 hours, or most preferably, at least 20 hours.

### Hepatitis B Virus

In one embodiment, the present invention relates to compositions (delivery vehicles) useful for preventing or treating Hepatitis B virus (HBV) and related disorders.

Thus, in one embodiment there is provided a composition (delivery vehicle) for modulating gene expression in a liver cell, the composition (delivery vehicle) including
a short interfering nucleic acid (siNA) molecule capable of modulating gene expression in a liver cell by RNA interference; and
a cationic hepatotropic lipid vector comprising DODAG (CDAN DOGS).

Modulating gene expression may be by inhibiting gene expression.

In one embodiment there is provided a composition (delivery vehicle) for modulating the inhibition of cellular proliferation in a liver cell, the composition (delivery vehicle) including
a short interfering nucleic acid molecule capable of modulating cellular proliferation in a liver cell by RNA interference; and
a cationic hepatotropic lipid vector comprising DODAG (CDAN DOGS).

Modulating cellular proliferation may be by inhibiting cellular proliferation.

In one embodiment there is provided a composition (delivery vehicle) for modulating viral replication in a liver cell, the composition (delivery vehicle) including
a short interfering nucleic acid molecule capable of modulating viral replication in a liver cell by RNA interference; and
a cationic hepatotropic lipid vector comprising DODAG (CDAN DOGS).

Modulating viral replication may be by inhibiting viral replication or viral gene expression.

The viral gene expression or replication may be Hepatitis B Virus (HBV) gene expression or Hepatitis B Virus (HBV) replication.

The liver cell may be a cultured liver cell, i.e. in vitro, or the liver cell may form part of a liver in a mammal, i.e. in vivo.

The short interfering nucleic acid molecule may be RNA, e.g siRNA. The short interfering RNA may be in the form of an siRNA duplex or double-stranded siRNA.

The short interfering nucleic acid molecule may include a nucleotide sequence complementary to HBV nucleic acid, or to a portion thereof. The HBV sequence may be derived from the sequence of GenBank accession code AY233274.

The short interfering nucleic acid duplex sequence may comprise SEQ ID NO. 1 or 2 (Table 1; siRNA 1407); a nucleic acid sequence complementary to SEQ ID NO. 1 or 2; a nucleic acid sequence which hybridizes specifically to SEQ ID NO. 1 or 2; a homologous sequence of a hepadnavirus; or a nucleic acid sequence which has at least 90% sequence identity to one of said sequences. In a preferred embodiment, the short interfering nucleic acid duplex sequence comprises SEQ ID NOs. 1 and 2.

The short interfering nucleic acid duplex sequence may comprise SEQ ID NO. 3 or 4 (Table 1); a nucleic acid sequence complementary to SEQ ID NO. 3 or 4; a nucleic acid sequence which hybridizes specifically to SEQ ID NO. 3 or 4; a homologous sequence of a hepadnavirus; or a nucleic acid sequence which has at least 90% sequence identity to one of said sequences. In a preferred embodiment, the short interfering nucleic acid duplex sequence comprises SEQ ID NOs. 3 and 4.

The short interfering nucleic acid duplex sequence may comprise SEQ ID NO. 5 or 6 (Table 1); a nucleic acid sequence complementary to SEQ ID NO. 5 or 6; a nucleic acid sequence which hybridizes specifically to SEQ ID NO. 5 or 6; a homologous sequence of a hepadnavirus; or a nucleic acid sequence which has at least 90% sequence identity to one of said sequences. In a preferred embodiment, the short interfering nucleic acid duplex sequence comprises SEQ ID NOs. 5 and 6.

The short interfering nucleic acid duplex sequence may comprise SEQ ID NO. 7 or 8 (Table 1); a nucleic acid sequence complementary to SEQ ID NO. 7 or 8; a nucleic acid sequence which hybridizes specifically to SEQ ID NO. 7 or 8; a homologous sequence of a hepadnavirus; or a nucleic acid sequence which has at least 90% sequence identity to one of said sequences. In

The short interfering nucleic acid duplex sequence may comprise SEQ ID NO. 9 or 10 (Table 1); a nucleic acid sequence complementary to SEQ ID NO. 9 or 10; a nucleic acid sequence which hybridizes specifically to SEQ ID NO. 9 or 10; a homologous sequence of a hepadnavirus; or a nucleic acid sequence which has at least 90% sequence identity to one of said sequences. In a preferred embodiment, the short interfering nucleic acid duplex sequence comprises SEQ ID NOs. 9 and 10.

The short interfering nucleic acid duplex sequence may comprise SEQ ID NO. 11 or 12 (Table 1; siRNA 1794); a nucleic acid sequence complementary to SEQ ID NO. 11 or 12; a nucleic acid sequence which hybridizes specifically to SEQ ID NO. 11 or 12; a homologous sequence of a hepadnavirus; or a nucleic acid sequence which has at least 90% sequence identity to one of said sequences. In a preferred embodiment, the short interfering nucleic acid duplex sequence comprises SEQ ID NOs. 11 and 12.

The short interfering nucleic acid duplex sequence may comprise SEQ ID NO. 13 or 14 (Table 1); a nucleic acid sequence complementary to SEQ ID NO. 13 or 14; a nucleic acid sequence which hybridizes specifically to SEQ ID NO. 13 or 14; a homologous sequence of a hepadnavirus; or a nucleic acid sequence which has at least 90% sequence identity to one of said sequences. In a preferred embodiment, the short interfering nucleic acid duplex sequence comprises SEQ ID NOs. 13 and 14.

TABLE 1
Anti-HBV siRNAs sequences

| | HBV | | SEQ |
|---|---|---|---|
| | target | | ID |
| SiRNA | sequence | siRNA sequence | NO: |
| 1407 | 1407-1429 | 5′- GCGGGACGUCCUUUGUUUACG -3′ | 1 |
| | | 3′- AGCGCCCUGCAGGAAACAAAU -5′ | 2 |
| 1410 | 1410-1432 | 5′- GGACGUCCUUUGUUUACGUCC | 3 |
| | | 3′- GCCCUGCAGGAAACAAAUGCA -5′ | 4 |
| 1741 | 1741-1763 | 5′- GGGGAGGAGAUUAGGUUAAAG -3′ | 5 |
| | | 3′- ACCCCCUCCUCUAAUCCAAUU -5′ | 6 |
| 1755 | 1755-1777 | 5′- GUUAAAGGUCUUUGUAUUAGG -3′ | 7 |
| | | 3′- UCCAAUUUCCAGAAACAUAAU -5′ | 8 |
| 1778 | 1778-1800 | 5′- GCUGUAGGCAUAAAUUGGUCU -3′ | 9 |
| | | 3′- UCCGACAUCCGUAUUUAACCA -5′ | 10 |
| 1794 | 1794-1816 | 5′- GGUCUGCGCACCAUCAUCAUG -3′ | 11 |
| | | 3′- AACCAGACGCGUGGUAGUAGU -5′ | 12 |
| 1795 | 1795-1817 | 5′- GUCUGCGCACCAUCAUCAUGC -3′ | 13 |
| | | 3′- ACCAGACGCGUGGUAGUAGUA -5′ | 14 |

The short interfering nucleic acid molecules may be at least 85% identical to one of said sequences mentioned above.

The siRNA duplex or double-stranded siRNA may have a 5′ overhang. The siRNA duplex or double-stranded siRNA may have a 3′ overhang. The overhang may be 2 nucleotides.

Each strand of the short interfering nucleic acid molecule may have a length in the range of 21 to 30 nucleotides. Preferably, the short interfering nucleic acid molecule has a length of 21 nucleotides.

The cellular proliferation may be cirrhosis or hepatocellular carcinoma.

In one embodiment, there is provided a composition (delivery vehicle) for inhibiting replication of Hepatitis B virus (HBV), which includes a short interfering nucleic acid (siNA) molecule capable of inhibiting HBV replication by RNA interference and a cationic hepatotropic lipid vector comprising DODAG (CDAN DOGS).

In one embodiment, there is provided a composition (delivery vehicle) comprising one or more siNA molecules and a cationic hepatotropic lipid vector comprising DODAG (CDAN DOGS) that modulates the expression of gene(s) encoding HBV and/or cellular proteins associated with the maintenance or development of HBV infection, cirrhosis or hepatocellular carcinoma.

In one embodiment there is provided the use of a composition (delivery vehicle) as described above in the manufacture of a medicament for the prevention or treatment of a virally caused liver disease.

In one embodiment there is provided the use of a composition (delivery vehicle) as described above for the manufacture of a medicament for the prevention or treatment of virally caused liver cellular proliferation.

In one embodiment there is provided the use of a composition (delivery vehicle) as described above in the manufacture of a medicament for the prevention or treatment of a HBV caused liver disease.

In one embodiment there is provided the use of a composition (delivery vehicle) as described above in the manufacture of a medicament for the prevention or treatment of HBV caused liver cellular proliferation.

In one embodiment there is provided the use of a composition (delivery vehicle) as described above for the prevention or treatment of a virally caused liver disease.

In one embodiment there is provided the use of a composition (delivery vehicle) as described' above for the prevention or treatment of virally caused liver cellular proliferation.

In one embodiment there is provided the use of a composition (delivery vehicle) as described above for the prevention or treatment of a HBV caused liver disease.

In one embodiment there is provided the use of a composition (delivery vehicle) as described above for the prevention or treatment of HBV caused liver cellular proliferation.

In one embodiment there is provided a substance or composition (delivery vehicle) for use in a method of preventing or treating a virally caused liver disease in a subject, said substance or composition (delivery vehicle) comprising a composition (delivery vehicle) as described above, and said method comprising administering an effective amount of said substance or composition (delivery vehicle) to said subject thereby to prevent or treat said liver disease.

In one embodiment there is provided a substance or composition (delivery vehicle) for use in a method of preventing or treating virally caused liver cellular proliferation in a subject, said substance or composition (delivery vehicle) comprising a composition (delivery vehicle) as described above, and said method comprising administering an effective amount of said substance or composition (delivery vehicle) to said subject thereby to prevent or treat said liver cellular proliferation.

In one embodiment there is provided a substance or composition (delivery vehicle) for use in a method of preventing or treating a HBV caused liver disease in a subject, said substance or composition (delivery vehicle) comprising a composition (delivery vehicle) as described above, and said method comprising administering an effective amount of said substance or composition (delivery vehicle) to said subject thereby to prevent or treat said HBV caused liver disease.

In one embodiment there is provided a method of preventing or treating a virally caused liver disease in a subject which includes administering an effective amount of a composition (delivery vehicle) as described above to said subject thereby to prevent or treat said liver disease.

In one embodiment there is provided a method of preventing or treating virally caused liver cellular proliferation in a subject which includes administering an effective amount of a composition (delivery vehicle) as described above to said subject thereby to prevent or treat said liver cellular proliferation.

In one embodiment there is provided a method of preventing or treating a HBV caused liver disease in a subject which includes administering an effective amount of a composition (delivery vehicle) as described above to said subject thereby to prevent or treat said HBV caused liver disease.

The cellular proliferation or disease may be cirrhosis or hepatocellular carcinoma.

In one embodiment there is provided a pharmaceutical composition (delivery vehicle) comprising a composition (delivery vehicle) as described above in an acceptable carrier or diluent.

In one embodiment there is provided a medicament comprising the composition (delivery vehicle) as described above.

In one embodiment, there is provided a method of modulating expression of a target gene in a liver cell comprising introducing an effective amount of a composition (delivery vehicle) into the cell, the composition (delivery vehicle) including a short interfering nucleic acid (siNA) molecule capable of modulating expression of said target gene in the liver cell by RNA interference and a cationic hepatotropic lipid vector comprising DODAG (CDAN DOGS), such that the siNA molecule modulates expression of the target gene.

Modulating expression of the target gene may be by inhibiting expression of the target gene.

By target is meant a sequence within a target RNA that is “targeted” for cleavage mediated by a siNA.

By “modulate” is meant the expression of the gene, such that expression is greater than or less than that observed in the absence of the modulator. The term modulate can mean inhibit, but the use of the word modulate is not limited to this definition.

In one embodiment there is provided a method of target validation for the assessment of modulation of gene expression in a liver cell, the method including the steps of providing a composition (delivery vehicle) including a short interfering nucleic acid (siNA) molecule capable of modulating gene expression in a liver cell by RNA interference and a cationic hepatotropic lipid vector comprising DODAG (CDAN DOGS), the siNA molecule including a sequence complementary to RNA of a target gene; introducing the composition (delivery vehicle) into a biological system under conditions suitable for modulating expression of the target gene in the biological system; and determining the function of the gene by assaying for any phenotypic change in the biological system.

In one embodiment there is provided a method of target validation for the assessment of the modulation of HBV gene expression in a liver cell, the method including the steps of providing a composition (delivery vehicle) including a short interfering nucleic acid (siNA) molecule capable of modulating gene expression in a liver cell by RNA interference and a cationic hepatotropic lipid vector comprising DODAG (CDAN DOGS), the siNA molecule including a sequence complementary to RNA of a target gene; introducing the composition (delivery vehicle) into a biological system under conditions suitable for modulating expression of the target gene in the biological system; and determining the function of the gene by assaying for any phenotypic change in the biological system.

In one embodiment there is provided a kit for use in a method of target validation for the assessment of the modulation of gene expression in a liver cell, the kit including a composition (delivery vehicle) as described above.

In one embodiment there is provided a kit for use in a method of target validation for the assessment of the modulation of HBV gene expression in a liver cell, the kit including a composition (delivery vehicle) as described above.

The kit may include suitable reagents and instructions.

### Modulating Expression or Activity

In certain embodiments of the present invention, a subject (e.g. a mammal, preferably, a human) has a condition that is amenable to gene silencing therapy. As used herein, “gene silencing therapy” refers to administration to the subject of a delivery vehicle comprising one or more agents—such as nucleic acid material encoding a therapeutic siRNA and subsequent expression of the administered nucleic acid material in situ (e.g. in vitro or in vivo). Thus, the phrase “condition amenable to siRNA therapy” embraces a variety of conditions—such as genetic diseases (ie., a disease condition that is attributable to one or more gene defects), acquired pathologies (ie., a pathological condition that is not attributable to an inborn defect), cancers, diseases and prophylactic processes (ie., prevention of a disease or of an undesired medical condition). A gene “associated with a condition” is a gene that is either the cause, or is part of the cause, of the condition to be treated.

As used herein, “acquired pathology” refers to a disease or syndrome manifested by an abnormal physiological, biochemical, cellular, structural, or molecular biological state. For example, the disease could be, for example, a viral disease—such as hepatitis—or cancer.

In one embodiment of the present invention, the term “modulating” means up-regulating, enhancing or increasing—such as up-regulating, enhancing or increasing the expression and/or activity of a nucleotide sequence.

In one embodiment of the present invention, the term “modulating” means down-regulating, supressing or decreasing—such as down-regulating, supressing or decreasing the expression and/or activity of a nucleotide sequence.

### Pharmaceutical Salt

The delivery vehicles may be administered in the form of a pharmaceutically acceptable salt.

Pharmaceutically-acceptable salts are well known to those skilled in the art. Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines such as diethanolamine, salts.

In one preferred aspect the lipid is provided in the form of a salt. Preferably the salt is a chloride, TFA, bromide, iodide or acetate salt. In one preferred aspect the salt is a tri-HCl salt.

The delivery vehicles may be administered as a pharmaceutically acceptable salt. Typically, a pharmaceutically acceptable salt may be readily prepared by using a desired acid or base, as appropriate. The salt may precipitate from solution and be collected by filtration or may be recovered by evaporation of the solvent.

### Pharmaceutical Compositions

Pharmaceutical compositions of the present invention may comprise a therapeutically effective amount of the delivery vehicles.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

If the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions may be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or the pharmaceutical compositions can be injected parenterally, for example, intravenously, intramuscularly or subcutaneously. For parenteral administration, the compositions may be best used in the form of a sterile aqueous solution which may contain other substances, for example, enough salts or monosaccharides to make the solution isotonic with blood. For buccal or sublingual administration the compositions may be administered in the form of tablets or lozenges which can be formulated in a conventional manner.

In one embodiment of the present invention it is preferred that the delivery vehicles or compositions described herein are administered orally.

The delivery vehicles may be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98155148.

The pharmaceutical composition comprising the delivery vehicles may also be used in combination with conventional disease treatments.

### Administration

The delivery vehicles may be administered alone but will generally be administered as a pharmaceutical composition—eg. when the delivery vehicles are in admixture with a suitable pharmaceutical excipient, diluent or carrier selected with regard to the intended route of administration and standard pharmaceutical practice.

For example, the delivery vehicles may be administered in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents—such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) may include, but are not limited to, one or more of oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

In one embodiment of the present invention, it is preferred that the route for administration (delivery) is parenteral (e.g. by an injectable form).

### Dose Levels

The delivery vehicles may be included in a pharmaceutical preparation in dosage units. This means that the preparations are in the form of individual parts, for example capsules, pills, suppositories and ampoules, of which the content of the liposome/delivery vehicle corresponds to a fraction or a multiple of an individual dose. The dosage units can contain, for example, 1, 2, 3 or 4 individual doses or a fraction of (e.g. ½, ⅓, or ¼, etc.) of an individual dose. An individual dose typically contains the amount of the liposome/delivery vehicle which is given in one administration and which usually corresponds to a whole, a half, a third, or a quarter of a daily dose.

The liposome/delivery vehicle may be present in a pharmaceutical preparation at a concentration of about 0.01 to 5 wt. %, about 0.05 to 1 wt. %, about 0.1 to 1.5 wt. %, about 0.2 to 1 wt. %, or about 0.5 to 1 wt. % relative to the total mixture.

In some cases, it may be necessary to deviate from these dosages and in particular to do so as a function of the nature and body weight of the subject to be treated, the nature and the severity of the illness, the nature of the preparation and if the administration of the medicine, and the time or interval over which the administration takes place.

Thus it can suffice in some cases to manage with less then these amounts of active compound, whilst in other cases the amounts of active compound may be exceeded.

In animal studies, a typical dosage is 1 to 2 mg/kg or less. Typically, a physician or veterinarian will determine the actual dosage, which will be most suitable for an individual subject.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

### Formulation

The delivery vehicles may be formulated into a pharmaceutical composition, such as by mixing with one or more of a suitable carrier, diluent or excipient, by using techniques that are known in the art.

A delivery vehicle comprising a liposomal formulation comprising paclitaxel and carboplatin as active agents may be used for treating lung cancer.

A liposomal formulation comprising two or more agents selected from irinotecan, paclictaxel, and carboplatin as active agents may be useful for treating patients with lung cancers—such as non-small cell lung carcinoma.

A liposomal formulation comprising gemcitabine and epirubicin as active agents may be useful for treating patients with urothelial carcinoma.

A liposomal formulation useful for treating ovarian carcinoma include a liposomal formulation comprising gemcitabine and cisplatin as active agents; a liposomal formulation comprising gemcitabine and carboplatin as active agents; a liposomal formulation comprising gemcitabine and paclitaxel as active agents; a liposomal formulation comprising gemcitabine and topotecan as active agents and a liposomal formulation comprising gemcitabine and doxorubicin as active agents. Two or more agents selected from the group consisting of gemcitabine, cisplatin, carboplatin, paclitaxel, topotecan, and doxorubicin may be used to treat ovarian carcinoma.

A liposomal formulation useful for treatment of melanoma includes a liposomal formulation comprising interleukin-2 and histermine dihydrochloride as active agents or tamoxifen, and cisplatin as active agents. Two or more agents selected from the group consisting of interleukin-2, histermine dihydrochloride, tamoxifen and cisplatin may be used to treat melanoma

A liposomal formulation useful for treatment of breast cancer includes a liposomal formulation comprising herceptin and paclitaxel as active agents. Another liposomal formulation comprises adriamycin, cytoxin, and herceptin as active agents. Another liposomal formulation comprises anastrozole and tamoxifen as active agents. Another liposomal formulation comprises proleukin and herceptin as active agents. Thus, two or more agents selected from the group consisting of herceptin, paclitaxel, adriamycin, cytoxin, anastrozole, tamoxifen and proleukin can be used to treat breast cancer.

A liposomal formulation useful for treatment of colorectal cancer includes a liposomal formulation comprising 5-fluorouricil, leucovorin, and oxaliplatin as active agents.

Another useful liposomal formulation comprises 5-irinotecan, 5-fluorouracil, and leucovorin as active agents. Another useful liposomal formulation comprises oxaliplatin and irinotecan as active agents. Another useful liposomal formulation comprises sulindac and EKI-569 as active agents. Two or more agents selected from the group consisting of 5-fluorouracil, leucovorin, oxaliplatin, 5-irinotecan, irinotecan, sulindac and EKI-569 may be used to treat colorectal cancer.

A liposomal formulation useful for treatment of liver cancer includes a liposomal formulation comprising Doxorubicin (Adriamycin), epidoxorubicin and/or cisplatin as active agents.

Suitable amounts of the agents used in the delivery vehicles described herein are those amounts that can be stably incorporated into the liposome/delivery vehicle. The agents may each be present in the liposome/delivery vehicle in amounts of from at least 1 to 50 wt. %—such as 2 to 25 wt. %.

The loading capacity of the liposomes/delivery vehicles is typically between about 90% to 100% of the agent(s).

If the lipid coupled polymer is not introduced into the liposome/delivery vehicle by post-modification but instead by pre-modification, the loading capacity is typically 50-60%.

Preferably, the liposomes/delivery vehicles are loaded with one or more agents at low pH, preferably, between about pH 3.5-4.5.

### Kits

The present invention also provides liposomes or delivery vehicles as described herein in kit form. The kit will typically be comprised of a container, which is compartmentalised for holding the various elements of the liposomes or delivery vehicles. The kit will contain the liposomes or delivery vehicles of the present invention, preferably in dehydrated form, with instructions for their rehydration and administration.

### Further Aspects

The liposomes/delivery vehicles described herein may be used to efficiently transfect cells—such as eukaryotic cells, in particular mammalian cells, with one or more agents—such as siRNA.

The liposomes/delivery vehicles described herein may be used for the oral delivery or administration of one or more agents—such as siRNA.

The liposomes/delivery vehicles described herein may be used to efficiently transfect one or more agents into the blood stream in order to treat blood directly or to access organs and tumours etc.

The liposomes/delivery vehicles may be used in a variety of applications—such as gene therapy, DNA vaccine delivery and in vitro transfection studies.

The delivery vehicles may be used in a variety of delivery applications—such as gene therapy, DNA vaccine delivery and in vitro transfection studies—of the blood.

The delivery vehicles may also be used to administer therapeutic genes to a patient suffering from a disease.

In a further aspect, there is provided a method for introducing one or more agents—such as siRNA—into a cell, said method comprising contacting said cell with a delivery vehicle as described herein.

In a further aspect, there is provided a method for silencing the expression of a nucleic acid sequence, said method comprising administering to a mammalian subject an effective amount of a therapeutically effective amount of a delivery vehicle as described herein.

In a further aspect, there is provided a method for the in vivo delivery of one or more agents—such as siRNA—said method comprising administering to a mammalian subject a delivery vehicle as described herein.

In a further aspect, there is provided a method for in vivo delivery of one or more agents—such as siRNA—to a cell—such as a liver cell—said method comprising administering to a mammalian subject a delivery vehicle as described herein.

In a further aspect, there is provided a method of treating a disease in a mammalian subject, said method comprising administering to said subject a therapeutically effective amount of a delivery vehicle as described herein.

In a further aspect, there is provided a method of treating a disease in a mammalian subject, said method comprising administering to said subject a therapeutically effective amount of a delivery vehicle as described herein, wherein said disease is associated with expression of a gene comprising a target sequence for said siRNA.

Preferred features and characteristics of one aspect of the invention are applicable to other aspects mutatis mutandis.

The invention will now be further described by way of Examples, which are meant to serve to assist one of ordinary skill in the art in carrying out the invention and are not intended in any way to limit the scope of the invention.

### DESCRIPTION OF THE FIGURES

FIG. 1: A. Cholesteryloxycarbonyl-3,7-diazanonane-1,9-diamine (CDAN) 1; dioleoyl L-α-phosphatidylethanolamine (DOPE) 2; cholesteryl-PEG³⁵⁰-aminoxy lipid (CPA) 3; cholesteryl-aminoxy lipid (CA) 4. B. Scheme showing synthesis of DODAG (or “CDAN-DOGS”). Reagents and conditions: i) PMe₃ plus H₂O in THF, r.t., 2.5 h., 83%; ii) Methylacrylate, MeOH, r.t., 12 h., 54%; iii) Boc₂O, NEt₃, CH₂Cl₂, r.t., 12 h, 25%; iv) LiOH, THF, 4° C., 12 h., 91%; v) Pd/C-10%, Boc₂O, MeOH, H₂, r.t., 12 h, 92%; yl) LiOH, THF, 4° C., 12 h., 97%; vii) HBTU, DMAP, CHCl₃, r.t., 12 h, 70%; viii) TFA, CH₂Cl₂, N₂, r.t., 2 h, 92%; ix) HBTU, DMAP, CHCl₃, r.t., 24 h, 62%; x) TFA, CH₂Cl₂, r.t., 3 h, 97%.

FIG. 2: Agarose gel shift assays with lipid:siRNA ratios between 0 and 1.8. All samples were vortexed and the whole reaction mixture pipetted to the individual wells of 0.8% pre-cast agarose gels and run at 50V/10 mA for 20 mins. Note that above lipid:siRNA ratios of 1.8, the siRNA was completely retarded in the well.

FIG. 3: Down-regulation of the cyclophilin B gene in vitro using CDAN, DODAG or Transfectam. 250,000 HeLa cells were seeded on 6-well plates and grown over night. A specific anti-cyclophilin siRNA (Dharmacon) was delivered using the three above mentioned lipids under optimal conditions for each lipid and the mRNA levels for CyPB analysed after 48 h using real time PCR.

FIG. 4: Down-regulation of the apoB gene in vivo. Female Balb/C mice were injected with a specific anti-apoB siRNA formulated with either CDAN or DODAG at 5 mg siRNA/kg animal or 15 mg siRNA/kg (CDAN only). The animals were sacrificed after 48 h, the livers extracted and immediately mashed in RNAlater (Ambion). The total RNA was extracted according to established protocols using the RNAeasy kit (Qiagen). 1 μg of total RNA for each well was reversed transcribed and 1/40 of the generated cDNA used for real time PCR (Sybr green). Data are plotted as normalized against β-actin.

FIG. 5: Serum concentrations of LDH 4 d post-administration of saline control or various DODAG-siRNA AB nanoparticle formulations to mice (relative to saline control, data were not significantly different).

FIG. 6: Serum concentrations of ALT 4d post-administration of saline control or various DODAG-siRNA AB nanoparticle formulations to mice (relative to saline control, data were not significantly different).

FIG. 7. Effect of DODAG-siRNA AB nanoparticle formulations on markers of HBV replication in murine hydrodynamic injection (MHI) models: Serum concentrations of HBsAg measured at 48 and 96 h in MHI mice treated with saline, DODAG-siRNA AB nanoparticle formulations or naked siRNAs by intravenous administration (single dose: 1 mg/kg siRNA per animal, tail vein injection) (relative to saline control, data were not significantly lower except for both DODAG-siRNA 1407 and DODAG-siRNA 1794 data; P values<0.05).

FIG. 8: Effect of DODAG-siRNA AB nanoparticle formulations on markers of HBV replication in murine hydrodynamic injection (MHI) models: Circulating viral particle equivalents (VPEs, virions/ml) measured at 96 h in MHI mice treated as in FIG. 7 (relative to naked siRNA control data, both DODAG-siRNA 1794 and DODAG-siRNA 1407 data were significantly lower; P values<0.05).

FIG. 9. Markers of viral replication and immunostimulation in HBV transgenic mice. Circulating VPEs measured at 28 d in HBV transgenic mice treated (every 3 d) with saline, DODAG-siRNA-AB nanoparticle formulations or naked siRNAs by intravenous administration over a period of 4 weeks (each dose: 1 mg/kg/day siRNA per animal, tail vein injection) in comparison to the HBsAg concentration at 28 d resulting from lamivudine administration (daily dose: 200 mg/kg/day, i.p.) (relative to saline control, only DODAG-siRNA 1407 data were significantly lower; P values<0.05).

FIG. 10: Markers of viral replication and immunostimulation in HBV transgenic mice. Intrahepatic concentrations of HBV mRNA isolated from HBV transgenic mice at 28 d treated as described in FIG. 9. Graphical representation to indicate the ratio of HBV surface to housekeeping glyceraldehyde 3-phosphate dehydrogenase (GAPDH) mRNA (relative to saline control, only DODAG-siRNA 1407 were significantly lower; P values<0.05).

FIG. 11: Markers of viral replication and immunostimulation in HBV transgenic mice. Intrahepatic concentrations of OAS1 and IFNβ mRNA 28 d post-administration of saline, siRNA 1407 alone, siRNA 1794 alone, DODAG-siRNA 1407, or DODAG-siRNA 1794. As positive control, mice were treated with poly I:C at 6 h before sacrifice, hepatic RNA extraction and measurement of OAS1 and IFN-β markers of interferon response induction (relative to saline control, all data were not significantly different except for Poly I:C data).

FIG. 12: A. Retardation gel assay: comparison of DODAG/DOPE (1:1 m/m) with CDAN/DOPE (1:1 m/m). B. Collodial stability of lipoplexes as a function of the cationic lipid-DNA weight ratio.

FIG. 13: In vitro transfection efficiency of pDNA-AB nanoparticles prepared from DODAG/DOPE (1:1, m/m) liposomes and luciferase expression plasmid (pCMV-luc): luciferase activity versus lipid/pDNA (w/w) ratio.

FIG. 14: Transfection activity on Hela cells with DODAG/DOPE 1:1 liposomes:cytotoxicity assay.

FIG. 15: In vivo transfection efficiency of pDNA-ABC nanoparticles formulated from cationic liposome DODAG/DOPE (1:1, m/m) combined with pDNA (pUMVC1-nt-β-gal) (lipid:pDNA ratio of 0.5 or 2, w/w) and added Chol-PEG⁵⁰⁰⁰ (Chol-PEG⁵⁰⁰⁰:pDNA ratio of 2, w/w): βgal activity; (a) non-treated; (b) CDAN/DOPE (w/w=2)/pDNA (25 μg)/Chol-PEG⁵⁰⁰⁰; (c) DODAG/DOPE (w/w=2)/pDNA (25 μg)/Chol-PEG⁵⁰⁰⁰; (d) invivojetPEI/pDNA (25 μg), N/P=8; (e) AdbetaGal, titer=10¹³, 1:50 dilution.

FIG. 16: In vivo gene delivery towards the mouse airway by intranasal instillation of pDNA-ABC nanoparticles formulated from cationic liposome DODAG/DOPE (1:1, m/m): in toto X-gal staining.

FIG. 17: In vivo gene delivery towards the mouse airway by intranasal instillation of pDNA-ABC nanoparticles formulated from cationic liposome DODAG/DOPE (1:1, m/m): histological analysis after X-gal staining (without counterstaining).

FIG. 18: A. In vivo gene delivery towards the mouse airway by intranasal instillation of pDNA-ABC nanoparticles formulated from cationic liposome DODAG/DOPE (1:1, m/m): histological analysis after X-gal staining (with Eosin counterstaining, low and intermediate power view). B. In vivo gene delivery towards the mouse airway by intranasal instillation of pDNA-ABC nanoparticles formulated from cationic liposome DODAG/DOPE (1:1, m/m): histological analysis after X-gal staining (with Eosin counterstaining, high power view).

FIG. 19: Levels of Itk in human primary T lymphocytes after treatment with siRNA-AB (1, 2, 4 and 6) and siRNA-ABC (3 and 5) nanoparticle formulations. CD4+ cells were isolated from freshly collected blood using negative selection and seeded at 5×10³ cells/well. Cells were incubated for 4 h together with particles in Optimem before full media were added on the cells. 24 h post-siRNA delivery cells were analysed with Taqman RT-PCR for Itk levels.

FIG. 20: GAPDH knockdown in Jurkat human T-lymphocytes. Cells were seeded at 10⁵ cells/well and transfection was allowed for 4 h in Optimem. Then cells were lysed and analysed for GAPDH content using a fluorimetric assay (KD-alert, Ambion) and a Varioskan plate fulorimeter.

FIG. 21: Effect of the charge (N/P) ratio on zeta potential of DODAG-containing siRNA ABC nanoparticles. DODAG formulations were prepared with 20 and 50 mol % and siRNA at various N/P ratios. Zeta potential measurements were measured using a Zetasizer.

FIG. 22: Effect of charge ratio on siRNA loading efficiency of DODAG-containing siRNA ABC nanoparticles. Unencapsulated siRNA % was calculated from YOYO-1 fluorimetric assays. All DODAG-containing siRNA ABC nanoparticles achieved encapsulation efficiency above 80%. Nanoparticles were prepared with 20 and 50 mol % of DODAG as appropriate.

FIG. 23: Effect of charge ratio on uptake of FAM-labeled siRNA ABC nanoparticles by DU145 cells after 2 and 4 h incubation assessed by FACS. Histograms were given a different color for different cell treatment.

Upper panels: line a represents free FAM-siRNA uptake at 2 and/or 4 h incubation. Upper and lower panels: line b=untreated cells; line c=cells treated with FAM-labeled siRNA ABC nanoparticles formulated with N/P ratio of 1.5:1; line d=cells treated with nanoparticles formulated with N/P ratio of 2:1; line e=cells treated with nanoparticles formulated with N/P ratio of 4:1; line f=cells treated with nanoparticles formulated with N/P ratio of 8:1.

FIG. 24: Liver and tumour sections after administration of FAM-siRNA 20 mol % DODAG-containing siRNA ABC nanoparticles (N/P 4:1). Animals were sacrificed at 24 h post i.v. injection. Upper left panel shows fluorescent signal related to Rhodamine-DOPE incorporated at 1 mol % in nanoparticles. Upper right panel shows fluorescence related to FAM-siRNA. Note DODAG-containing siRNA ABC nanoparticle appear to be abundant in Kupffer cells. Lower left panel shows fluorescent signal related to Rhodamine-DOPE in the tumour section. Lower right panel shows fluorescent signal related to FAM-labeled siRNA (arrow).

### EXAMPLES

### Example 1

### Synthesis

### Synthesis of H₂N—(CH₂)₂—HN—(CH₂)₃—HN—(CH₂)₂—C(═O)NH—CH₂—C(═O)—N[(CH₂)₁₇CH₃]₂ (“DODAG”/“CDAN-DOGS”)

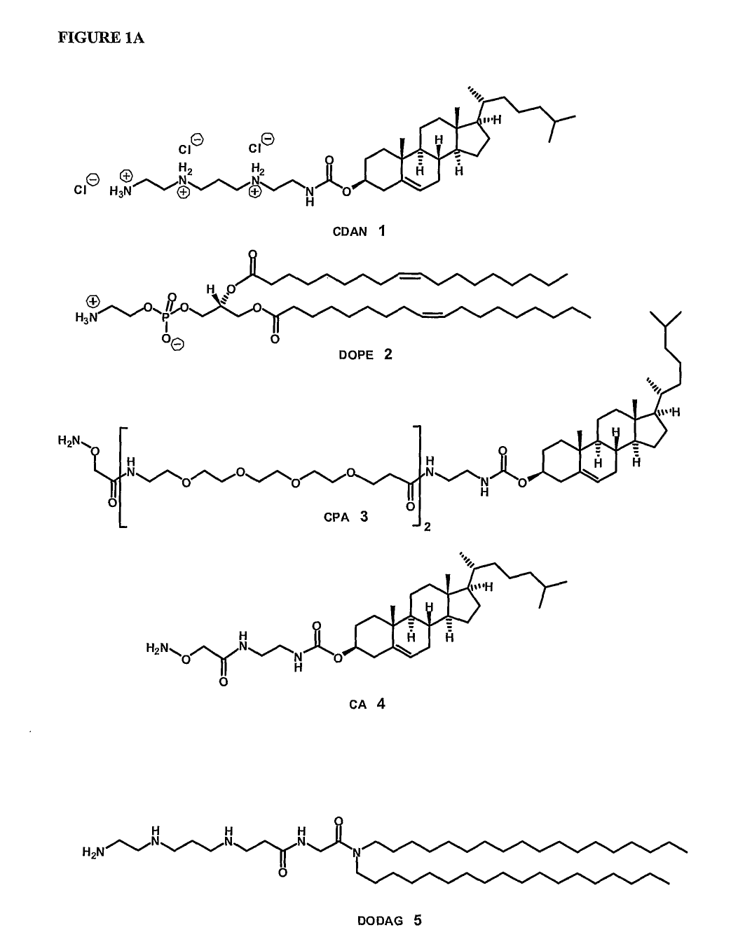

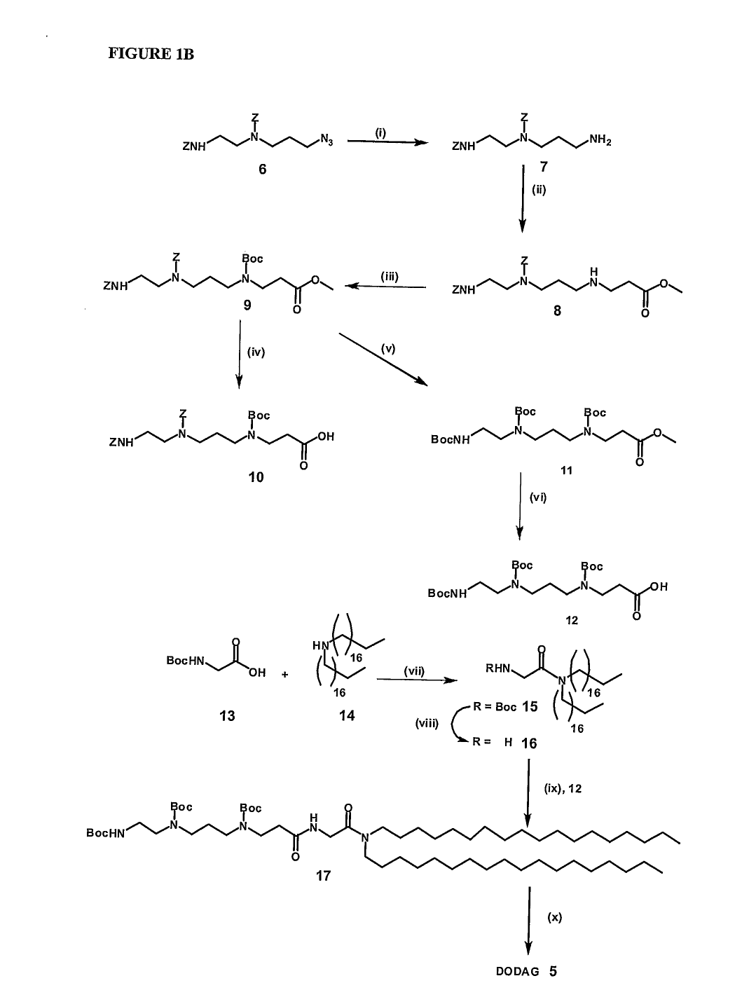

The synthesis of DODAG 5 was completed in two stages as shown (FIG. 1B; Z=benzyloxycarbonyl; BOC=tert-butyloxycarbonyl). The azide 6, readily available from a previous synthesis of CDAN 1 (Keller, M., Jorgensen, M. R., Perouzel, E. and Miller, A. D. Thermodynamic aspects and biological profile of CDAN/DOPE and DC-Chol/DOPE lipoplexes, Biochemistry, 42, 6067-6077 (2003)), was smoothly converted into polyamine 7 using a trimethylphosphine adaptation of the Staudinger reaction. Afterwards, a Michael addition of polyamine 7 to methylacrylate was used to give methyl ester 8 in moderate yield. N-Boc protection of 8 then resulted in methyl aminodecanoate 9 that was subject to methyl ester hydrolysis to give the acid 10. Compound 10 was initially seen as a useful synthon for DODAG 5 synthesis, but the mixed protecting groups proved difficult to completely remove. Therefore, 9 was subject alternatively to protecting group exchange resulting in the uniformly protected ester 11. This was followed by methyl ester hydrolysis to give the uniformly protected aminodecanoic acid 12. In the second stage of DODAG 5 synthesis, N-Boc-glycine 13 was converted using dioctadecylamine 14 into tertiary N-Boc-glycine amide 15 and then α-N-deprotection was carried out to give the key tertiary glycine amide 16 intermediate, in excellent yield. Conjugation of 16 with uniformly protected aminodecanoic acid 12 gave fully protected DODAG 17 and final deprotection resulted in DODAG 5 in excellent yield. This was lyophilised in several cycles from aqueous acetonitrile giving the desired compound.

### Synthesis of Me₂N—(CH₂)₂—NMe—(CH₂)₃—NMe—(CH₂)₂—C(═O)NH—CH₂—C(═O)—N[(CH₂)₁₇CH₃]₂

Methylation of the amino groups of DODAG was achieved by treatment with a formaldehyde/formic acid mixture at elevated temperature. The product was obtained in 51% yield as a hydroscopic white powder after workup, column chromatography and lyophilization and shown to be of analytical grade purity.

### Detailed Description of Synthesis Methods

### General Synthesis

All chemicals were purchased from Sigma Aldrich (Dorset, UK) unless otherwise stated. Dried dichloromethane was distilled with phosphorus pentoxide; other solvents were purchased pre-dried or as required from Sigma-Aldrich (Dorset, UK) or BDH Laboratory Supplies (Poole, UK). HPLC-grade acetonitrile was purchased from Fisher Chemicals (Leicester, UK) and other HPLC-grade solvents from BDH Laboratory Supplies (Poole, UK). Thin layer chromatography (TLC) was performed on pre-coated Merck-Kieselgel 60 F₂₅₄ aluminium backed plated and revealed with ultraviolet light, iodine, acidic ammonium molybdate (IV), acidic ethanolic vanillin, or other agents as appropriate. Flash column chromatography was accomplished on Merck-Kieselgel 60 (230-400 mesh). Mass spectra were recorded using Bruker Esquire 3000, VG-7070B or JEOL SX-102 instruments. ¹H- and ¹³C-NMR were recorded on Advance Brucker 400 Ultrashield™ machine using residual isotopic solvent as an internal reference (s=singlet, d=doublet, t=triplet, q=quartet, quin=quintet, br=broad singlet). Analytical HPLC (Hitachi-LaChrom L-7150 pump system equipped with a Polymer Laboratories PL-ELS 1000 evaporative light scattering detector) was conducted with a Vydac C4 peptide column with gradient 0.1% aqueous trifluoroacetic acid (TFA) to 100% acetonitrile (0.1% TFA) [0-15 min.], then 100% acetonitrile (0.1% TFA) [15-25 min], then 100% methanol [25-45 min].

### 4,6-benzyloxycarbonyl-4-aza-1,6-hexane diamine 7

Trimethylphosphine in THF (1 M, 3 mL, 3 mmol) was added to a solution of azide 6 (950 mg, 2.3 mmol) in THF (28 mL) under argon. The reaction was stirred at room temperature and monitored by TLC, until completion (˜2.5 h). A solution of water (4.2 mL) and NH₃ (aq) (4.2 mL) was added to the reaction mixture. The reaction was stirred at room temperature for a further 1 h. The solvents were removed in vacuo rendering a yellow oil. This was purified by silica gel column chromatography (NH₃:MeOH:CH₂Cl₂ 0.6:4.2:95.2 to 1:7:92, v/v/v). Evaporation of the solvents afforded the desired product (721 mg, 83%) as a yellow oil; FTIR (film) υₘₐₓ 3355, 2931, 2093, 1651, 1524, 1455, 1247, 1139 cm⁻¹; ¹H NMR (CDCl₃) δ 1.45-1.51 (2H, m, CH₂CH₂NH₂), 1.52-1.61 (2H, m, CH₂CH₂NH₂) 2.65-2.45 (2H, m, NH₂), 3.1-3.35 (6H, m, CH₂CH₂N(Cbz)CH₂), 5.05 (2H, s, PhCH₂O), 5.2 (2H, s, PhCH₂O), 5.35-5.7 (1H, m, NHamide), 7.10-7.35 (10H, m, aromatic); ¹³C NMR (CDCl₃) δ 33.4 (CH₂CH₂CH₂), 39.6 (CH₂NH₂), 40.5 (NCH₂CH₂N), 42.1 (NCH₂CH₂CH₂), 46.1 (NCH₂CH₂N), 66.3 (PhCHH₂O), 67.7 (PhCH₂O), 126.2-127.3 (10×CH, aromatic), 137.6 (2×C), 157.1 (2×C═O); m/z (ESI+ve) 386 (M+H), 342 (CbzHN(CH₂)₂N(Cbz)-); FAB-MS m/z calculated for C₂₁H₂₈N₃N₄ (M+H) 386.2092, found 386.2079.

### Methyl 8,10-benzyloxycarbonyl-4,8-diaza-10-aminodecanoate 8

Polyamine 7 (144.6 mg, 0.38 mmol) was dissolved in methanol (7 mL) and stirred at room temperature under argon. Methylacrylate (37.2 μL, 0.41 mmol) was added to the reaction and the resulting solution was stirred at room temperature. The progress of reaction was monitored by means of TLC. When the reaction had gone to completion (˜12 h) the solvent and excess methylacrylate were removed in vacuo to rendering a dark yellow oil. This was purified by silica gel column chromatography (MeOH:Ethylacetate:CH₂Cl₂ 2:3:15 to 4:3:13, v/v/v). Evaporation of the solvents afforded the desired product (91.5 mg, 54%) as a yellow oil; ¹H NMR (CDCl₃) δ 1.7-1.85 (2H, m, CH₂CH₂CH₂), 2.3-2.55 (4H, m, CH₂NHCH₂), 2.65-2.8 (2H, m, CH₂COOCH₃), 3.1-3.35 (6H, m, CH₂CH₂N(Cbz)CH₂), 3.60 (3H, s, OCH₃), 4.95 (1H, s, PhCH₂O), 5.05 (1H, s, PhCH₂O), 5.15-5.45 (1H, m, NH amide), 7.15-7.32 (10H, m, aromatic); ¹³C NMR (CDCl₃) δ 34.7 (CH₂CH₂CH₂), 40.3 (CH₂COOCH₃), 45.3-47.5 (CH₂CH₂N(Cbz) CH₂CH₂CH₂N(Cbz) CH₂), 52.0 (OCH₃), 67.0 (OCH₂Ph), 67.7 (OCH₂Ph), 128.9-128.3 (10×CH, aromatic), 136.91 (2×C), 156.89 (2×COOCH₂Ph), 173.59 (COOMe); m/z (ESI+ve) 472 (M+H).

### Methyl 4-tert-butyloxycarbonyl-8,10-benzyloxycarbonyl-4,8-diaza-10-aminodecanoate 9

To a solution of methyl ester 8 (150 mg, 0.32 mmol) and Boc₂O (69.5 mg, 0.32 mmol) in dry CH₂Cl₂ (80 mL) was added triethylamine (133 μL, 0.96 mmol). The progress of the reaction was monitored by means of TLC. When the reaction had reached completion (˜12 h), the reaction mixture was poured onto saturated NaHCO₃ solution (20 mL). This was then poured into H₂O (50 mL). The organic layer was separated and the aqueous layer washed with CH₂Cl₂ (3×100 mL). The combined organic layers were dried (MgSO₄). The solvents were removed in vacuo rendering a yellow oil. The oil was purified by silica gel column chromatography [Petroleum ether:Diethylether 8:2 to 2:8, v/v]. Evaporation of solvents afforded the desired product (45 mg, 25%) as a yellow oil; ¹H NMR (CDCl₃) δ 1.40 (9H, s, (CH₃)₃), 1.53-1.72, (2H, m, CH₂CH₂CH₂), 2.33-2.52 (2H, m, CH₂COOCH₃), 2.91-2.52 (10H, m, NHCH₂CH₂N(Cbz)CH₂CH₂CH₂N(Cbz)CH₂), 3.60 (3H, s, OCH₃), 4.9-5.05 (2H, m, 2×PhCH₂O), 5.10-5.57 (1H, m, NH amide), 7.2-7.35 (10H, m, aromatic); ¹³C NMR (CDCl₃) δ 28.8 (C(CH₃)₃), 30.1 (CH₂CH₂CH₂), 34.1 (CH₂COOCH₃), 40.5-46.1 (CH₂CH₂N(Cbz) CH₂CH₂CH₂N(Cbz) CH₂), 52.1 (OCH₃), 66.9 (2×OCH₂Ph), 67.7 (C(CH₃)₃), 128.3-128.9 (10×CH, aromatic), 136.9 (2×C, aromatic), 155.6 (NHCO), 157.0 (COBoc, COCbz), 172.0 (COOMe); m/z (ESI+ve) 572 (M+H).

### 4-tert-butyloxycarbonyl-8,10-benzyloxycarbonyl-4,8-diaza-10-aminodecanoic acid 10

Methyl aminodecanoate 9 (15 mg, 0.03 mmol) was dissolved in THF (3 mL) and LiOH solution 10% (w/v) (3 mL) was added. The resulting solution was stirred at 4° C. until TLC indicated the reaction had gone to completion (˜12 h). The reaction mixture was poured into 10% citric acid solution (w/v) (3 mL), and subsequently extracted with CH₂Cl₂ (3×10 mL) and CHCl₃:MeOH 2:1, v/v (2×10 mL). The combined organic layers were back extracted with water (3×10 mL). The organic layers were combined, dried (MgSO₄) and concentrated in vacuo, rendering the title compound (13 mg, 91%) as a yellow oil; ¹H NMR (CDCl₃) δ 1.43 (9H, s, C(CH₃)₃), 1.57-1.73 (2H, m, CH₂CH₂CH₂), 2.4-2.62 (2H, m, CH₂CO₂H), 3.01-3.52 (10H, m, CH₂CH₂N(Cbz)CH₂CH₂CH₂N(Boc)CH₂), 4.93-5.17 (4H, m, 2×OCH₂Ph), 7.20-7.56 (10H, m, aromatic); m/z (ESI+ve) 579 (M+Na).

### Methyl-4,8,10-tert-butyloxycarbonyl-4,8-diaza-10-aminodecanoate 11

Methyl aminodecanoate 9 (113 mg, 0.198 mmol) was dissolved in MeOH (10 mL). Pd/C—10% (22.6 mg, 20% eq. w/w) and Boc₂O (107.8 mg, 0.245 mmol) were added to the solution. The reaction mixture was stirred at room temperature under H₂ (g), until TLC indicated the reaction had gone to completion (˜12 h). Pd/C—10% was removed by filtration over celite, and the filtrate was concentrated in vacuo. The resulting residue was dissolved in CH₂Cl₂ (40 mL) and extracted with water (3×40 mL). The combined aqueous extracts were back extracted with CH₂Cl₂ (3×20 mL) and CHCl₃:MeOH 2:1, v/v (2×40 mL). The organic extracts were combined, dried (MgSO₄) and concentrated. The resulting oil was purified by silica gel column chromatography [Petroleum ether:Diethylether 1:1→Petroleum ether:Diethylether 1:3, v/v], giving the title compound (91.5 mg, 92%) as a yellow oil: FTIR υₘₐₓ 3358, 3024, 2975, 2925, 1739, 1695, 1597, 1518, 1479, 1452, 1416, 1390, 1366, 1249, 1166, 1071 cm⁻¹; ¹H NMR (CDCl₃) δ 1.23-1.59 (27H, m, Boc CH₃'s), 1.61-1.79 (2H, m, CH₂CH₂CH₂), 2.43-2.59 (2H, m, CH₂CO₂H), 3.02-3.30 (8H, m, CH₂N(Boc)CH₂CH₂CH₂N(Boc)CH₂), 3.31-3.42 (2H, m, BocNHCH₂), 3.60 (3H, s, OCH₃); m/z (ESI+ve) 504 (M+H).

### 4,8,10-tert-butyloxycarbonyl-4,8-diaza-10-aminodecanoic acid 12

Uniformly protected ester 11 (93 mg, 0.185 mmol) was dissolved in THF (3 mL) and LiOH solution 10% (w/v) (3 mL) was added. The resulting solution was stirred at 4° C. until TLC indicated the reaction had gone to completion (˜12 h). The reaction mixture was poured into 10% citric acid solution (w/v) (30 mL), and subsequently extracted with CH₂Cl₂ (3×30 mL) and CHCl₃:MeOH 2:1, v/v (2×30 mL). The combined organic layers were back extracted with water (3×30 mL). The organic layers were combined, dried (MgSO₄) and concentrated in vacuo, rendering the title compound (88 mg, 97%) as a yellow oil; ¹H NMR (CDCl₃) δ 1.31-1.57 (27H, m, Boc CH₃), 1.63-1.75 (2H, m, CH₂CH₂CH₂), 2.45-2.57 (2H, m, CH₂CO₂H), 3.08-3.29 (8H, m, CH₂N(Boc)CH₂CH₂CH₂N(Boc)CH₂), 3.39-3.41 (2H, m, BocNHCH₂); ¹³C NMR (CDCl₃) δ 27.2 (9 C3×Boc C(CH₃)₃), 32.7 (CH₂CH₂CH₂), 38.2 (CH₂COOH), 46.5-42.7 (CH₂CH₂N(Boc)CH₂CH₂CH₂N(Boc)CH₂), 78.3 (2×NCOOC(CH₃)₃), 78.7 (NCOOC(CH₃)₃), 154.2 (NCOOC(CH₃)₃), 154.9 (NCOOC(CH₃)₃), 155.3 (NCOOC(CH₃)₃), 174.2 (COOH); m/z (ESI+ve) 490 (M+H).

### N′,N′-dioctadecyl-N-tert-butyloxycarbonyl-glycine amide 15

N-Boc-glycine 13 (307 mg, 1.75 mmol) and dioctadecylamine 14 (915 mg, 1.75 mmol) was dissolved in dry chloroform (30 mL) under anhydrous conditions. 2-(1H-benzotriazol-1-yl)-1,1,3,3-tetramethyluronium hexafluorophosphate (HBTU) (797.6 mg, 2.10 mmol) and 4-dimethylaminopyridine (DMAP) (642.3 mg, 5.26 mmol) were added to the solution. The reaction was stirred at room temperature under argon until TLC indicated the reaction had gone to completion (˜12 h). Solvents were removed in vacuo. The residue dissolved in CH₂Cl₂ (50 mL) and extracted with water (3×50 mL). The combined aqueous extracts were back extracted with CH₂Cl₂ (3×50 mL) and CHCl₃:MeOH 2:1, v/v (2×50 mL). The organic extracts were combined, dried (MgSO₄) and concentrated. The resulting yellow oil was purified by silica gel column chromatography (Petroleum→Petroleum:Diethylether 8:2, v/v), rendering the title compound (801 mg, 70%) as a yellow oil; FTIR (film) υₘₐₓ 3422, 2923, 2855, 2250, 1704, 1647, 1498, 1465, 1368, 1248, 1170, 1098 cm⁻¹; ¹H NMR (CDCl₃) δ 0.78-0.91 (6H, m, 2×CH₃), 1.1-1.34 (60H, m, alkylchain CH₂'s), 1.35-1.60 (13H, m, C(CH₃)₃ and N(CH₂CH₂-alkylchain)₂), 3.01-3.12 (2H, m, NCH₂), 3.23-3.41 (2H, m, NCH₂), 3.85-3.96 (2H, m, NHCH₂CO), 5.50-5.59 (1H, br, s, amide NH); ¹³C NMR (CDCl₃) δ 14.5 (2×CH₃), 23.1 (N(CH₂CH₂-alkylchain)₂), 27.3-32.3 (30 CH₂, alkylchain), 42.5 (NHCH₂CO), 46.5 (N(CH₂CH₂-alkylchain)₂), 47.3 (N(CH₂CH₂-alkylchain)₂), 79.8 (C(CH₃)₃), 156.2 (C(CH₃)₃COCO), 167.9 (CON-alkylchains); m/z (ESI+ve) 679 (M+H).

### N,N-dioctadecyl-glycine amide 16

N-Boc-glycine amide 15 (260 mg, 0.38 mmol) was dissolved in dry CH₂Cl₂ (5 mL) under anhydrous conditions. Trifluoroacetic acid (3 mL) was added to the solution cautiously. The reaction was stirred at room temperature under a positive flow of nitrogen until TLC indicated the reaction had gone to completion (˜2 h). The solvents were removed in vacuo rendering the desired compound (200 mg, 92%) as an off-white solid: FTIR (nujol mull) υₘₐₓ 2914, 1732, 1667, 1589, 1463, 1376, 1318, 1275, 1209, 1181, 1125 cm⁻¹; ¹H NMR (CDCl₃) δ 0.85-0.92 (6H, t, 2×CH₃), 1.17-1.43 (60H, m, 30 CH₂'s alkylchain), 1.45-1.60 (4H, m, N(CH₂CH₂-alkylchain)₂), 3.01-3.11 (2H, m, NCH₂CH₂-alkylchain), 3.21-3.3 (2H, m, N(CH₂CH₂-alkylchain), 3.85-3.95 (2H, m, CH₂NH₂); ¹³C NMR (CDCl₃) δ 14.5 (2×CH₃), 23.1 (2×CH₂CH₃), 27.1-32.4 (30 CH₂, alkylchain), 40.5 (CH₂NH₂), 46.9 (N(CH₂CH₂-alkylchain), 47.6 (N(CH₂CH₂-alkylchain), 165.5 (C═O); m/z (FAB+ve) 579 (M⁺); FAB MS calculated for C₃₈H₇₉N₂O (M+H) 579.6194, found 579.6192.

### N′,N′-dioctadecyl-N-4,8,10-tert-butyloxycarbonyl-4,8-diaza-10-aminodecanoylglycine amide 17

Uniformly protected aminodecanoic acid 12 (88 mg, 0.18 mmol) and tertiary glycine amine 16 intermediate (104 mg, 0.18 mmol) was dissolved in dry chloroform (10 mL) under anhydrous conditions. DMAP (77 mg, 0.63 mmol) and HBTU (88.6 mg, 0.23 mmol) were added to the solution. The reaction was stirred at room temperature under argon and progress was monitored by means of TLC, After 24 h the reaction had reached completion. The solvents were removed in vacuo and the remaining residue was dissolved in CH₂Cl₂ (50 mL) and extracted with water (3×50 mL). The combined aqueous extracts were back extracted with CH₂Cl₂ (3×50 mL) and CHCl₃:MeOH 2:1, v/v (2×50 mL). The organic layers were combined, dried (MgSO₄) and concentrated. The resulting yellow oil was purified by silica gel column chromatography (Petroleum ether:Diethylether 3:7→Diethylether:ethyl acetate 9:1, v/v), affording the title compound (110 mg, 62%) as a yellow oil: FTIR. (film) υₘₐₓ 3398, 2923, 2854, 2251, 1771, 1684, 1642, 1508, 1467, 1392, 1367, 1249, 1164, 1072 cm⁻¹; ¹H NMR (CDCl₃) δ 0.79-0.83 (6H, t, 2×CH₃ (J=6.7 Hz)), 1.14-1.33 (60H, m, Alkylchain CH₂'s), 1.36-1.49 (31H, m, Boc C(CH₃)₃'s and N(CH₂CH₂-alkyl chain)₂), 1.63-1.69 (2H, m, CH₂CH₂CH₂), 2.42-2.46 (2H, m, CH₂CH₂CONH), 3.07-3.40 (14H, m, BocNHCH₂CH₂N(Boc)CH₂CH₂CH₂N(Boc)CH₂ and N(CH₂CH₂-alkyl chain)₂), 3.41-3.49 (2H, m, BocNHCH₂CH₂), 3.93-3.97 (2H, m, CH₂CONAlkylchain); ¹³C NMR (CDCl₃) δ 13.0 (2×CH₃), 21.6 (2×CH₂CH₃), 25.8 (CH₂CH₂CH₃), 25.9 (CH₂CH₂CH₃), 26.5 ((Boc)NCH₂CH₂CH₂N(Boc), 27.0-29.2 (26 CH₂'s Alkyl chains and 3×C(CH₃)₃), 30.8 ((Boc)NCH₂CH₂CONH), 33.1 ((Boc)NCH₂CH₂CONH), 34.7 (N(CH₂CH₂-alkyl chain)₂), 38.5 (BocNHCH₂), 40.1 (NHCH₂N-alkyl chains), 42.6 ((Boc)NCH₂CH₂CH₂N(Boc)), 44.2, 44.7 ((N(CH₂-alkyl chain)₂), 45.1 ((Boc)NCH₂CH₂CH₂N(Boc)), 45.8 ((Boc)NHCH₂CH₂), 77.9 (NHCOOC(CH₃)₃), 78.60, 78.7 (NCOOC(CH₃)₃), 154.2 (NHCOOC(CH₃)₃), 155.0 (2×NCOOC(CH₃)₃), 166.0 (CONHCH₂), 169.8 (CON(Alkylchains)); m/z (FAB+ve) 1051 (M+H); FAB-MS m/z calculated for C₆₁H₁₁₉N₅O₈ (M+H) 1050.9124, found 1050.9136.

### N′,N′-dioctadecyl-N-4,8-diaza-10-aminodecanoylglycine amide (DODAG(“CDAN-DOGS”)) 5

Fully protected DODAG 17 (100 mg, 0.095 mmol) was dissolved in dry CH₂Cl₂ (20 mL) under anhydrous conditions. Trifluoroacetic acid (7 mL) was added to the solution cautiously. The reaction was stirred under a positive flow of nitrogen until TLC indicated that the reaction had gone to completion (˜3 h). The solvents were removed in vacuo rendering a pale solid. This was re-dissolved in CH₂Cl₂ (20 mL) and concentrated in vacuo repeatedly (×8). The resulting solid was then freeze-dried from acetonitrile:water 1:1, v/v, giving the title compound (70 mg, 97%) as a white fluffy powder: FTIR (nujol mull) υₘₐₓ 3289, 2920, 2852, 1669, 1463, 1376, 1198, 1169, 1137 cm⁻¹; ¹H NMR (CD₃OD) δ 0.80 (6H, t, 2×CH₃ (J=7.2 Hz)), 1.14-1.31 (60H, m, 30×CH₂-alkyl chains), 1.40-1.47 (2H, m, N(CH₂CH₂-alkylchain), 1.48-1.56 (4H, m, N(CH₂CH₂-alkylchain), 2.02-2.10 (2H, quin, NHCH₂CH₂CH₂NH (J=7.7 Hz)), 2.63-2.66 (2H, t, NHCH₂CH₂CONH (J=6.2 Hz)), 3.06-3.12 (4H, m, N(CH₂CH₂-alkylchain)₂), 3.18-3.27 (10 H, m, H₂NCH₂CH₂NHCH₂CH₂CH₂NHCH₂), 3.99 (2H, s, br, NHCH₂CO); ¹³C NMR (CD₃OD) δ 14.9 (2×CH₃), 24.2 (2×CH₂CH₃), 24.7 (2×CH₂CH₂CH₃), 29.0-31.2 (26 CH₂'s Alkyl chains), 32.3 (NHCH₂CH₂CH₂NH), 33.5 (H₂NCH₂), 37.5 (NHCH₂CH₂CO), 42.3 (N(CH₂CH₂-alkyl chain)₂), 45.5 (N(CH₂CH₂-alkyl chain)₂), 46.3 (NHCH₂CH₂CO), 46.4 (NHCH₂CH₂CH₂NH), 46.8 (NHCH₂CH₂CH₂NH), 48.1 (NHCH₂CO), 48.8 (H₂NCH₂CH₂), 170.5 (CONH), 172.8 (CON-alkyl chains); m/z (FAB+ve) 750 (M⁺); FAB-MS m/z calculated for C₄₆H₉₅N₅O₂ (M⁺) 750.7549, found 750.7564.

### N′,N′-dioctadecyl-N-4,8-dimethyldiaza-10-dimethylaminodecanoylglycine amide (permethyl DODAG)

DODAG 5 (44 mg, 0.0587 mmoles) was stirred in a 25 mL round bottom flask in an ice bath and then formic acid (3 mL) was added drop-wise. Then, aqueous formaldehyde solution (3 mL) was also added and after removal of the ice bath, the resulting mixture was refluxed gently for 3 h. The solution was concentrated in vacuo and the resulting residue was taken up in NaOH (5M, 3 mL) and extracted into CH₂Cl₂ (5×8 mL) followed by drying over MgSO₄, The organic layer was concentrated in vacuo affording a slightly yellow oily residue that was purified by flash column chromatography using a dichloromethane/methanol/water affording the pure permethyl DODAG in 51% yield. Mass spectrometry [M+H]=806.5 and [M−H]=804.7 and by HPLC showing a peak with a retention time=28.13 min (99% purity). ¹H NMR (400 MHz, CDCl₃) δ=0.87 (t, 6H, J=6.8 Hz, 2×CH₃), 1.26 (m, 60H, 30×CH₂), 1.47-1.52 (m, 4H, 2×CH₂), 1.72 (sept, 2H, CH₂), 2.25-2.35 (m, 12H, 4×NCH₃), 2.4-2.6 (m, 10H, 5×CH₂), 2.67 (t, 2H, J=8 Hz, CH₂), 3.17 (t, 2H, J=8 Hz, CH₂), 3.31 (t, 2H, J=8 Hz, CH₂), 4.05 (d, 2H, J=4 Hz, CH₂), 8.25 (t, 1H, J=4 Hz, NH). ¹³C NMR (400 MHz, CDCl₃) δ=14.10 (CH₃), 22.67 (CH₂), 26.88 (CH₂), 26.91 (CH₂), 27.05 (CH₂), 27.64 (CH₂), 28.77 (CH₂), 29.35-29.69 (CH₂), 31.91 (CH₂), 33.28 (CH₂), 41.28 (CH₂), 41.39 (CH₃), 42.25 (CH₃), 46.20 (CH₂), 46.96 (CH₂), 53.49 (CH₂), 55.15 (CH₂), 55.36 (CH₂), 56.12 (CH₂), 56.92 (CH₂), 167.44 (C═O), 172.32 (C═O); FAB-MS+ found [M+H]⁺, 806.815018, C₅₀H₁₀₄N₅0₂ required [M+H]⁺, 806.819003. HPLC analysis: t_{R}=28.1 min, column Vydac C-4 peptide: gradient H₂O (0.1% TFA)/MeCN (0.1% TFA)/MeOH, 0 min [100/0/0], 1-15 min [0/100/0], 25 min [0/100/0], 25.1 min [0/0/100], 45 min [0/0/100], 45.1 min [100/0/0], 55 min [100/0/0]; flow 1 mL/min.

### Examples 2-8

### General Methodology

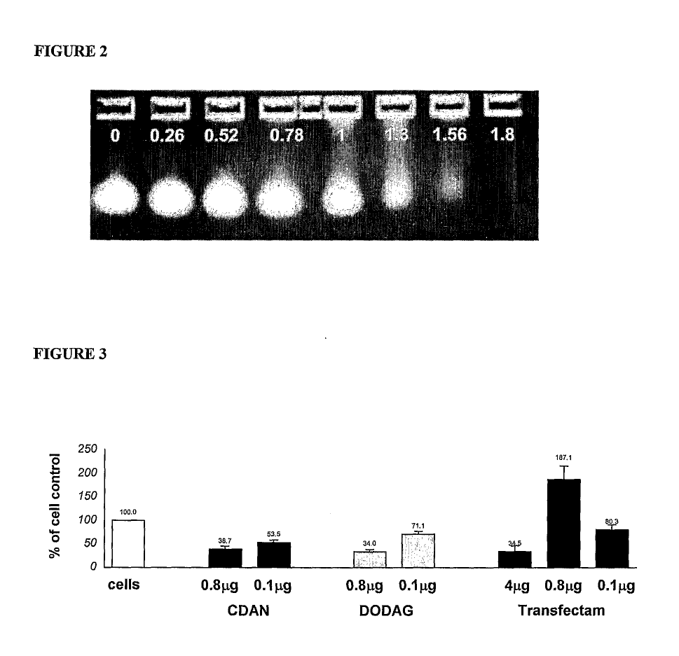

Gel retardation assay. In order to define the optimal ratio between lipid:siRNA to achieve full complexation of the nucleic acid which is manifested by a complete retardation in an electrical field, different ratios of lipid:siRNA (0-3) were produced and aliquots run on precast 0.8% agarose gels (Invitrogen). For this purpose, 1 μg siRNA were pipetted into 0.5 mL eppendorf tubes complemented with distilled water to 10 μL. The appropriate amount of lipid from a stock in water (2 mg/mL) were added to generate lipid:siRNA ratios between 0 and 3. All samples were vortexed and the whole reaction mixture pipetted to the individual wells of 0.8% pre-cast agarose gels and run at 50V/10 mA for 20 mins (FIG. 2). As can be seen from FIG. 2, complete retardation of the siRNA is achieved above ratios of 1.8 lipid:siRNA. Therefore, for all future in vivo experiments, a ratio of 3:1 (lipid:siRNA) was chosen.

In vivo assessment of efficacy of anti HBV siRNAs. The murine hydrodynamic tail vein injection (MHI) method was initially employed to determine the effects of siRNAs on the expression of HBV genes in vivo. Experiments on animals were carried out in accordance with protocols approved by the University of the Witwatersrand Animal Ethics Screening Committee. A saline solution comprising 10% of the mouse's body mass was injected via the tail vein over 5-10 seconds. The saline solution included a combination of 1 μg target DNA (pCH-9/3091) or 10 μg pLTR β-gal (Wang, C. & Stiles, C. D. Platelet-derived growth factor alpha receptor gene expression: isolation and characterization of the promoter and upstream regulatory elements. Proc. Natl. Acad. Sci. USA 91, 7061-7065 (1994), a control for hepatic DNA delivery, which encodes constitutively active β-galactosidase marker gene). Post-hydrodynamic injection (8 h), DODAG (or “CDAN-DOGS”) siRNA-AB nanoparticles (tail vein injection [approx 200 μl]; dose 1 mg/kg siRNA per animal) were injected under low pressure via the tail vein. Control mice were treated with naked siRNA solution (tail vein injection [approx 200 μl]; dose 1 mg/kg siRNA per animal) in which the siRNA was not complexed to non-viral delivery vehicles. Blood was collected from the tail vein over a period of 5 days and HBsAg was measured using the electrochemiluminescence assay (ECLIA) from Roche Diagnostics (Mannheim, Germany) according to the manufacturer's instructions. Animals were sacrificed after 4 days.

Markers of HBV replication in vivo. To measure effects of DODAG siRNA-AB nanoparticle formulations on circulating virion DNA, total DNA was isolated from 50 μl of the serum of mice on days 3 and 5 after hydrodynamic injection and viral particle equivalents determined using qPCR according to previously described methods (Carmona, S. et al. Effective inhibition of HBV replication in vivo by anti-HBx short hairpin RNAs. Mol. Ther. 13, 411-421 (2006)) with EuroHep calibrating standards (Heermann, K. H., Gerlich, W. H., Chudy, M., Schaefer, S. & Thomssen, R. Quantitative detection of hepatitis B virus DNA in two international reference plasma preparations. Eurohep Pathobiology Group. J. Clin. Microbiol. 37, 68-73 (1999)). Measurement of concentrations of mRNA encoding HBV and IFN response-related genes was also measured according to previously described methods (Carmona, S. et al. Effective inhibition of HBV replication in vivo by anti-HBx short hairpin RNAs. Mol. Ther. 13, 411-421 (2006)). All real time PCRs were carried out using the Roche Lightcycler V.2. Controls included water blanks and RNA extracts that were not subjected to reverse transcription. Taq readymix with SYBR green (Sigma, Mo., USA) was used to amplify and detect DNA during the reaction. Thermal cycling parameters consisted of a hotstart for 30 sec at 95° C. followed by 50 cycles of 58° C. for 10 sec, 72° C. for 7 sec and then 95° C. for 5 sec. Specificity of the PCR products was verified by melting curve analysis and agarose gel electrophoresis. Fixed and unfixed frozen liver sections were processed respectively for immunohistochemical HBV Core antigen (HBcAg) detection or for β-galactosidase staining (Sanes, J. R., Rubenstein, J. L. & Nicolas, J. F. Use of a recombinant retrovirus to study post-implantation cell lineage in mouse embryos. EMBO J. 5, 3133-3142 (1986)). A rabbit polyclonal antibody against HBcAg (Signet Laboratories Inc., MA, USA) and horseradish peroxidase-conjugated secondary antibody (Dako, Denmark) were used to detect the viral antigen in paraffin embedded sections according to standard procedures.

Experimental Toxicity. NMR1 mice were injected with DODAG siRNA-AB nanoparticle solutions into the tail vein as previously described. Four days after the injection, the mice were anaesthetized, and blood samples collected by cardiac puncture before sacrifice. The blood samples were submitted for haematological analysis, urea and electrolyte concentration determination, alanine transaminase (ALT) and lactate dehydrogenase (LDH) activity determination. Assays were performed in the accredited Haematology and Chemical Pathology Department laboratories of the South African National Health Laboratory Services (NHLS) in Johannesburg.

HBV Transgenic mice. HBV transgenic mice with greater than genome length HBV sequence stably integrated into their genomes that constitutively generate HBV particles (Marion, P. L. et al. in Frontiers in Viral Hepatitis pp 197-202 (Elsevier Science Amsterdam, 2003)) were used to assess anti viral efficacy of formulations. All procedures were approved by the Animal Care Committee at Stanford University. DODAG siRNA-AB nanoparticle formulations were prepared as described above and injected via the tail vein (all siRNA doses: 1 mg/kg/day per animal added at the indicated days). Serum HBsAg was measured using a quantitative sandwich ELISA from Abbott Laboratories, and HBeAg was determined using the electrochemiluminescence assay (ECLIA) from Roche Diagnostics (Mannheim, Germany) according to the manufacturer's instructions. Circulating viral particle equivalents were determined using real time PCR according to procedures described above.

Statistical Analysis. Data are expressed as the mean±standard error of the mean. Statistical difference was considered significant when P<0.05 and was determined according to the Dunnett's multiple comparison test and calculated with the GraphPad Prism software package (GraphPad Software Inc., CA, USA).

### Example 2

DODAG (or “CDAN-DOGS”) was formulated with an siRNA (5′-GGAAAGACUGLTUCCAAAA; SEQ ID NO. 24) specific against murine & human CyPB (accession no X58990, murine; CR456829, human) at a lipid:siRNA ratio of 13:1 (w/w). Lower lipid:siRNA ratios proved unsuccessful in mediating specific down-regulation in vitro, despite total retardation of the siRNA on agarose gel above ratios of 1.8:1 (FIG. 2). DODAG-mediated siRNA delivery in DODAG-siRNA AB nanoparticles was compared with CDAN- and with DOGS-(Transfectam, Promega)-mediated delivery in equivalent lipid-siRNA AB nanoparticles. For CDAN, a lipid:siRNA ratio of 13:1 was used which was found to be optimal for in vitro, and for DOGS (Transfectam), a ratio of 5:1 was used as recommended by the manufacturer. Therefore, for the head to head comparison, each of the three lipids was used to deliver siRNA at the optimal conditions.

Experimental 4 μg (3.74 μL) siRNA were complemented with 770 μL OptiMEM and 52 μg (26 μL) DODAG (2 mg/mL, water) added under vortexing. The DODAG-siRNA AB nanoparticles were allowed to stand for 5 mins at room temperature. 200 μL of the DODAG-siRNA AB nanoparticle solution were pipetted to two wells of a six well plate containing HeLa cells (250,000), and the cells incubated at 37° C. for 3 h under CO₂ regime. The cells were then washed with warm PBS and incubated for another 48 h before lysing and extracting the total RNA using RNAeasy extraction columns (Qiagen). The same protocol was employed with CDAN. For DOGS, the protocol as recommended by the manufacturer was applied. The quality of the total RNA was verified on a 2% agarose formaldehyde gel, and the RNA quantified by UV (260 nm). 1 μg RNA of each sample was reverse transcribed to generate cDNA which was real time PCRed using standard protocols with Sybr green. The CyPB levels were standardized to β-actin levels for each sample (FIG. 3).

### Example 3

General The mouse apoB gene (accession no M35186) was targeted with a specific siRNA sequence 5′-GUCAUCACACUGAAUACCAU-3′ (SEQ ID NO: 25) that has previously been demonstrated to be an efficient siRNA to downregulate the apoB gene (Soutschek, J. et al. Therapeutic silencing of an endogenous gene by systemic administration of modified siRNAs. Nature 432, 173-178 (2004)).

Lipid-siRNA AB nanoparticles with CDAN and DODAG were prepared at final lipid:siRNA ratios of 2.5 and 3, respectively. These values correspond to the optimal ratios to completely retard the siRNA on a precast 0.8% agarose gel (Invitrogen). All lipid-siRNA AB nanoparticle complexes were made in water at a final concentration of 0.5 mg siRNA/mL in water.

Experimental The appropriate amounts of CDAN and DODAG from stock solutions in water (5 mg/mL and 2 mg/mL, respectively) were pipetted into a 25 mL falcon plastic tube. The appropriate amount of anti-apoB siRNA from a stock solution of 10 mg/mL was added slowly to the respective lipid solution while vortexing the falcon tube. The appropriate amount of a solution of trehalose (100%, w/v in water) was added and the sample vortexed heavily for 10 s. The samples were then sonicated in a water bath for 15 mins to generate homogenous emulsions. 15 mins prior to the injection of the samples into Balb/C mice, 1.5M NaCl was added so that each lipid-siRNA AB nanoparticle preparation contained a final concentration of 150 mM NaCl. 200 μL aliquots were injected via tail vein and the animals left for 48 h before sacrificing. The liver was extracted and immediately put into a solution of RNAlater (Ambion). The total RNA was extracted using RNAeasy columns (Qiagen), and quantified both by UV absorption (λ=260 nm) and by densiometry of the bands on a 2% agarose formaldehyde gel.

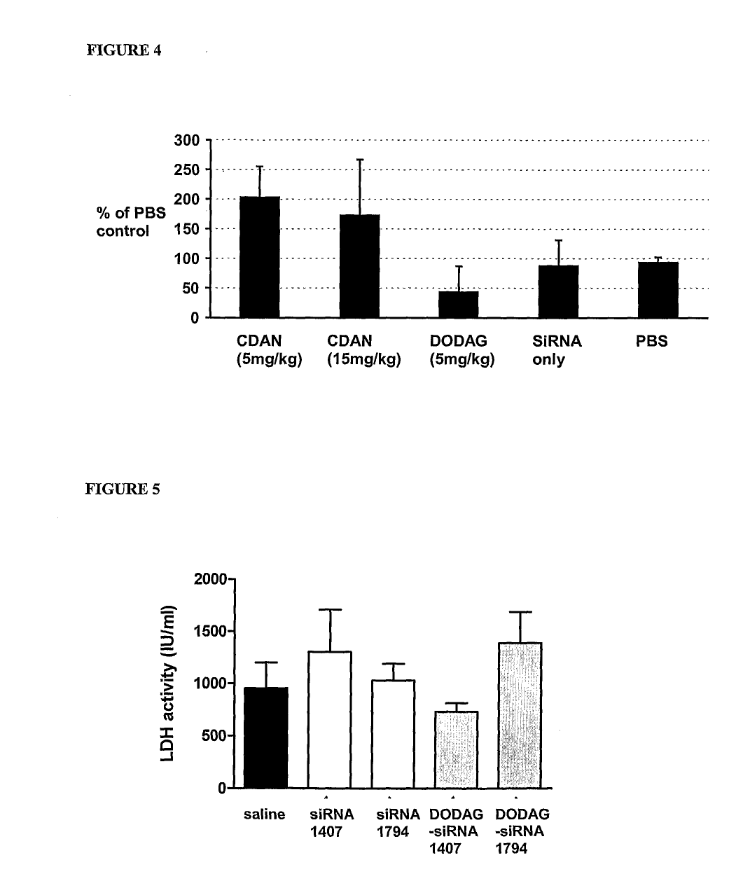

1 μg of the total RNA was reversed transcribed to generate cDNA in a total volume of 20 μL. After completion of the reaction, the total reaction volume was supplemented to 40 μL with water. From this solution, 1 μL was used to do real time PCR (Sybr green) using the forward primer 5′-GCCATGGGCAACTTTACCTATGA (SEQ ID NO: 26) and the reverse primer 5′-CTGCAGGGCGTCAGTGACAAATG (SEQ ID NO:27) on an Applied Biosystem 7700 machine. The data were analysed using the ABI prism software (FIG. 4). As can be seen from FIG. 4, DODAG-mediated delivery effected efficient down-regulation of the apoB mRNA in vivo (>60%) at a dose of 5 mg siRNA/kg animal, whereas CDAN at both 5 and 15 mg siRNA/kg animal did not mediate down-regulation, but rather surprisingly, up-regulation of the apoB gene.

### Example 4

### Formation of Micelles

The formation of DODAG-siRNA AB nanoparticles was achieved with relative ease. siRNA dispersed in water (1 mg/ml) was combined with an appropriate aliquot of DODAG 5 aqueous solution (2 mg/ml) by rapid vortex mixing (final lipid:siRNA ratio was 3:1 w/w), followed by bath sonication (15 min) in the presence of the osmolyte trehalose (5% w/v final concentration). This lipid:siRNA ratio translates into a cytofectin to nucleotide molar ratio, [cyt]/[nt], of 1.25, and hence an N/P ratio of approx. 1.8 assuming that each DODAG 5 molecule possesses a cationic charge of 1.5 at neutral pH, in line with the behaviour of the parent molecule CDAN 1 (Keller, M., Jorgensen, M. R., Perouzel, E. and Miller, A. D. Thermodynamic aspects and biological profile of CDAN/DOPE and DC-Chol/DOPE lipoplexes, Biochemistry, 42, 6067-6077 (2003)). Agarose gel retardation assays showed that an N/P ratio of 1.8 was that at which the movement of siRNA was entirely retarded, consistent with its complete encapsulation by DODAG 5 (FIG. 2). The modest excess positive charge was hoped to be sufficient to encourage hepatotrophism of the AB nanoparticles, but low enough to be masked by the presence of the osmolyte trehalose to obviate colloidal instability on the way to the liver post injection. In effect, the trehalose was intended to provide a “bolus”-stabilisation effect in serum, akin to a temporary stealth/biocompatibility polymer layer (C-layer) surrounding AB core particles in the blood and shielding these particles from immediate serum effects. Accordingly DODAG-siRNA AB nanoparticles may also be described as siRNA AB+C nanoparticles. Nanoparticle sizes are dependent on the duration of sonication, but typically 70±20 nm (as determined by photon correlation spectroscopy post preparation) was selected given the target organ preference. Nanoparticle suspensions were either lyophilized post formulation and reconstituted in saline prior to use, or else could be prepared fresh, supplemented with saline solution and i.v. injected immediately.

### Example 5

### Delivery Vehicle Hepatotropism and Experimental Toxicology Assessment

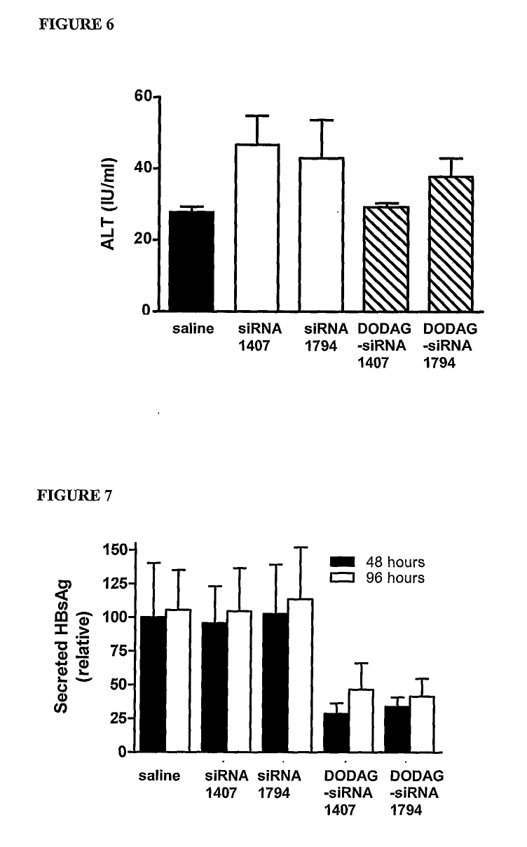

Previously, pharmacokinetic studies were performed with [¹⁴C]-labeled siRNA-ABC nanoparticles injected i.v. (1 mg/kg siRNA, tail vein) with variable levels of incorporation of PEG²⁰⁰⁰ (0.1-5.0 mol %) and these accumulated in the liver within 1 h (>85% of that injected) (Carmona, S. et al. Controlling chronic HBV replication in vivo with ‘tailor-made’ siRNA-ABC nanoparticles, in submission (2007)). Fluorescence microscopy with anti-GFP siRNA, 5′-labeled with cyanine dye Cy3 (red) was used to confirm delivery of siRNA to the liver, mostly to murine hepatocytes but also to Kupfer cells (liver macrophages). In general, the subcellular localization of 5′-Cy3-labelled siRNA within hepatocytes in vivo appeared similar to that observed in cells in vitro post siFECTamine® mediate delivery of 3′-fluorescein-labelled siRNA (Spagnou, S., Miller, A. D. & Keller, M. Lipidic Carriers of siRNA: Differences in the Formulation, Cellular Uptake, and Delivery with Plasmid DNA. Biochemistry 43, 13348-13356 (2004)). We considered it reasonable to expect DODAG-siRNA AB nanoparticles to have similar dynamic properties, at least during the first hour post-injection. This assumption could be proven through the analyses of toxicity and siRNA functional effects in vivo. With respect to toxicity, we examined for the serum activity of lactate dehydrogenase (LDH) a general marker of cell lysis (FIG. 5) and of alanine transaminase (ALT), a liver specific indicator of hepatocyte damage (FIG. 6). Except in the case of DODAG siRNA-1794 AB nanoparticles, neither of these activities was elevated significantly in the sera of either naked siRNA or nanoparticle treated animals compared with controls, The DODAG siRNA-1794 AB nanoparticles may indeed be somewhat toxic (see later results). However, renal function was not significantly affected by the delivery vehicles as determined by an absence of any significant perturbation in the concentrations of urea, creatinine and electrolytes compared with controls. Furthermore, the assessment of stained liver sections and peripheral blood smears from animals that had been treated regularly with DODAG-siRNA AB nanoparticles for a 4 week period did not show evidence for harmful effects.

### Example 6

### Antiviral Efficacy of siRNAs in Culture and in Murine Hydrodynamic Injection Models of Viral Replication

Anti-HBV siRNA sequences had already been screened in vitro, yielding two chemically unmodified sequences siRNA 1407 and siRNA 1794 that appeared to be of significant utility (Carmona, S. et al. Controlling chronic HBV replication in vivo with ‘tailor-made’ siRNA-ABC nanoparticles, in submission (2007)). The capacity of DODAG siRNA-AB nanoparticles for the effective delivery of these functional siRNAs was examined in a manner parallel to the previous study.

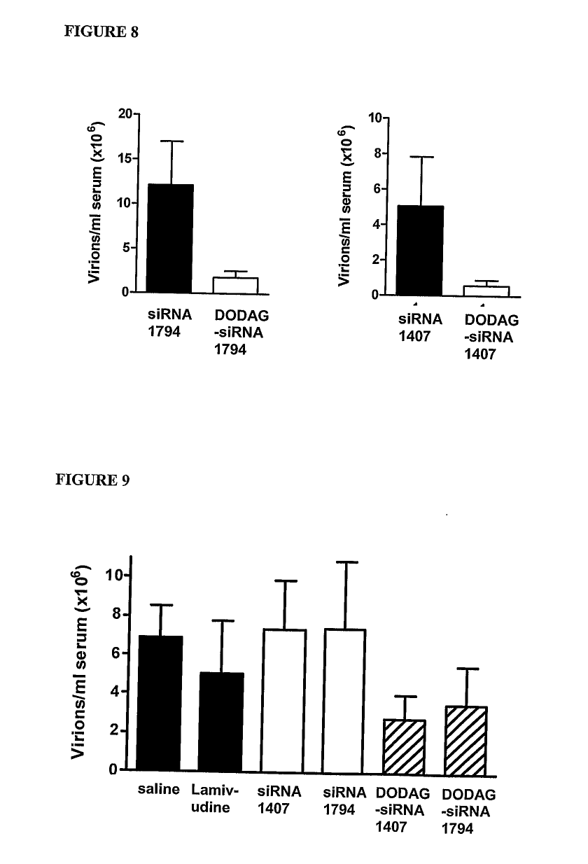

In order to assess HBV gene knockdown in vivo, a murine hydrodynamic injection (MHI) model of viral replication was employed initially. Efficiency of liver delivery of HBV replication competent plasmid (pCH-9/3091) were shown to be equivalent between MHI mice, as determined by staining for β-galactosidase activity expressed from a co-injected reporter plasmid (pLTR β-gal) (data not shown). At 8 h post MHI-mediated delivery of HBV genome, DODAG siRNA-AB formulations and naked siRNA samples were administered i.v. (single dose: 1 mg/kg siRNA, tail vein injection) then hepatitis B virus surface antigen (HBsAg) levels and viral particle equivalents were measured thereafter. Compared to controls, administration of siRNA 1407 or siRNA 1794 within DODAG siRNA-AB nanoparticles resulted in significant knockdown of serum levels of HBsAg at time points of 2 and 4 d post i.v. administration (FIG. 7). This inhibitory effect was confirmed by a significant knockdown of circulating viral particle equivalents (VPEs, virions/ml) at 96 h after hydrodynamic injection (FIG. 8). As noted previously, these results (FIGS. 7 and 8) were obtained by administration of a single dose of siRNA within 24 h of MHI-mediated delivery of HBV genome, thereby ensuring that delivered siRNA would be operating against a substantial background of transgene expression, akin to an acute HBV infection scenario in the murine liver (Carmona, S. et al. Controlling chronic HBV replication in vivo with ‘tailor-made’ siRNA-ABC nanoparticles, in submission (2007)). Overall, siRNA delivered in DODAG siRNA-AB nanoparticles appears to outperform siRNA delivered in our original siRNA-ABC nanoparticles, at least where treatment of HBV in this acute model of infection is concerned.

### Example 7

### Antiviral Efficacy of DODAG (“CDAN-Dogs”)-Mediated Delivery of siRNA in HBV Transgenic Mice

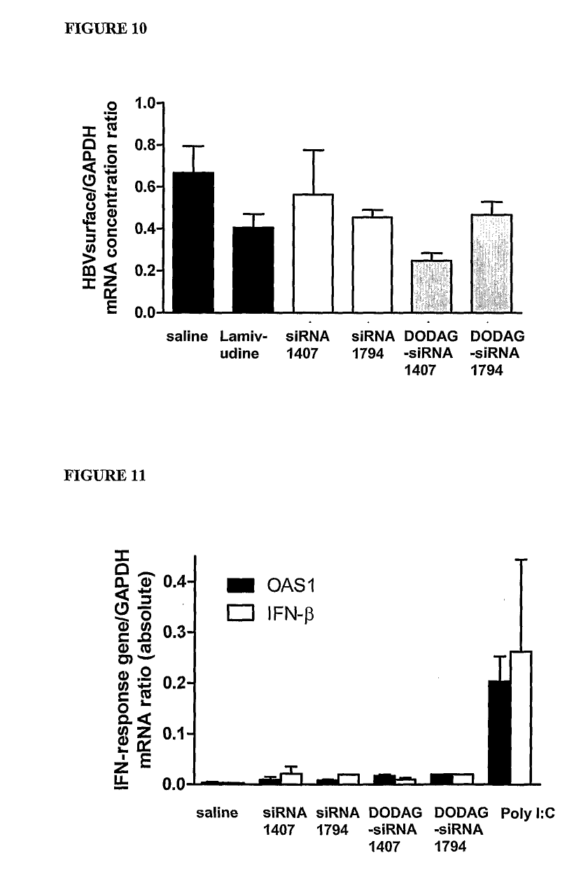

To assess knockdown in a model of HBV replication that more closely resembles the situation in patients who are chronic carriers of HBV, non-viral delivery vehicle formulations were also administered to HBV transgenic mice. These animals had been propagated after stable integration of greater than genome length HBV sequences (Marion, P. L. et al. in Frontiers in Viral Hepatitis pp 197-202 (Elsevier Science Amsterdam, 2003)). HBV particles are constitutively produced and the number of circulating viral particle equivalents is in the range from 0.5-1.0×10⁷ per ml. The DODAG siRNA-AB formulations and naked siRNA samples were administered i.v. every third day over a 4 week period (each dose: 1 mg/kg siRNA, tail vein injection). Both siRNA 1407 and 1794 nanoparticles resulted in a minimal decrease in HBsAg blood levels (at 28 days), as compared with saline administered controls. Similar observations were made with respect to Hepatitis B E-Antigen (HBeAg) levels. However a suppression of VPEs by 2-3 fold relative to controls at 28 d was seen with the administration of the DODAG siRNA-AB formulation containing siRNA 1407, a result that appears statistically significant (FIG. 9). The DODAG siRNA-AB formulation containing siRNA 1794 appeared to perform similarly but results were at the border of statistical significance compared with control data. Overall, the DODAG siRNA-AB nanoparticle mediated delivery of siRNA appeared to have an impact on VPE levels similar to that of lamivudine administration (FIG. 9). With respect to the knock-down of HBsAg mRNA in liver as determined by quantitative RT-PCR measurements (FIG. 10), data obtained with DODAG siRNA-AB formulation containing siRNA 1407 was consistent with a statistically meaningful reduction of 2-3 fold in mRNA message in liver, coupled with a statistically meaningful improvement on the performance of lamivudine administration.

### Example 8

### (Negative Control)—Interferon Response In Vivo, Oligoadenylate Synthase-1 (OAS-1) and Interferon-IFN-β)

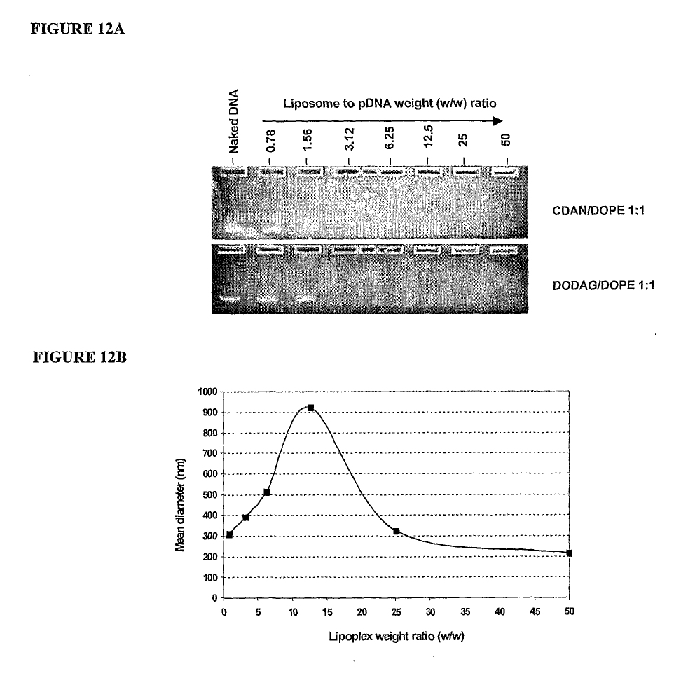

mRNA concentrations were measured in the livers of treated transgenic mice to determine whether there was evidence for significant activation of the interferon (IFN) response caused by siRNA administration (FIG. 11). The positive control was a group of mice that had been treated for 6 h with poly I:C, an inducer of the IFN response, using the hydrodynamic injection procedure. Comparisons to controls revealed that both siRNAs administered within DODAG siRNA-AB nanoparticles did not induce significant activation of the IFN response genes. This supports an interpretation that inhibitory effects on markers of HBV replication were probably not due to any unintended toxic immuno-stimulatory IFN response mechanisms.

### Example 9

### Preparation of DODAG/DOPE (pDNA)-AB Delivery Vehicles

### Preparation of Cationic Liposome Formulation.

For the preparation of DODAG/DOPE cationic liposomes, a mixture of cationic lipid DODAG and DOPE (1:1, m/m) in chloroform was prepared, and the solvent evaporated under vacuum (rotary evaporator). The thin film of DODAG cationic lipid and DOPE was then resuspended in a 5% Glucose buffer solution (pH 7.4) to give a final total lipid concentration of 5 mg/mL. The mixture was subsequently sonicated (Langford ultrasonics Sonomatic®) for 60 min to form small sized liposomes and filtered through a 0.22 μm filter (Millex GS, Millipore). Liposomes of around 100-120 nm were obtained and stored at 4° C. prior to use.

### Preparation of Plasmid DNA-AB Delivery Vehicles for Cellular Transfection In Vitro.

A plasmid DNA (pDNA)-AB nanoparticle delivery vehicle (also known generally as a cationic liposome-pDNA [LD; lipoplex] system) was prepared for in vitro transfection of cells in the following way. For each transfection well, plasmid DNA (1 μg/well) (pCMV-Luc) and the desired amount of cationic lipsomes (from above, 5 mg/ml starting concentration) were each diluted into separate aliquots of Dulbecco's modified Eagle's medium (DMEM) (50 μL) without fetal calf serum (FCS) and vortex-mixed. These two solutions were then combined, and the resulting pDNA-AB nanoparticle delivery vehicle solution was incubated for 30 min at ambient temperature prior to administration to cells.

Size determination was performed using dynamic light scattering analysis conducted on a N4 Plus (Beckman Coulter) apparatus at a detection angle of 90°. Mean particle diameters were determined by multimodal fit analysis.

Results. DODAG-containing cationic liposomes were formulated from the widely used neutral helper lipid DOPE. DODAG/DOPE (1:1, m/m) cationic liposome interactions with pDNA were investigated by gel retardation assay in comparison to commercially available CDAN/DOPE (1:1, m/m) (FIG. 12A). In both cases, pDNA mobility was completely retarded once a lipid:pDNA ratio of 4:1 w/w was reached. The complexation between DODAG/DOPE (1:1, m/m) cationic liposomes and pDNA resulted in a three zone model of colloidal stability as function of lipid:pDNA weight ratios in different pDNA-AB nanoparticles (FIG. 12B). In the first zone (lipid:pDNA weight ratio ranging from 0 to 2 w/w) nanoparticles possessed a mean diameter of 200-300 nm. In the second zone (lipid:pDNA ratio 2 to 7.5 w/w), nanoparticles increased to a mean diameter of 900 nm. In the third zone (lipid:pDNA ratio 7.5 to 25 w/w), nanoparticle mean diameter decreased to 200-300 nm once more.

### Example 10

### Preparation of DODAG/DOPE (pDNA)-ABC Delivery Vehicles

Preparation of plasmid DNA-ABC delivery vehicles for lung transfection in vivo. A plasmid DNA (pDNA)-ABC nanoparticle delivery vehicle was prepared for in vivo gene transfection of mouse airways as follows. DODAG/DOPE (1:1, m/m) cationic liposomes (5 mg/ml) were prepared in neutral medium (5% Glucose, pH 7.4) and then combined with Chol-PEG⁵⁰⁰⁰ and pDNA (pUMVC1-nt-βgal) in appropriate amounts (as indicated). The resulting pDNA-ABC nanoparticle delivery vehicle solution was then incubated for 30 min at ambient temperature prior to administration to animals (final pDNA concentration 0.25 mg/ml; 250 μg/ml).

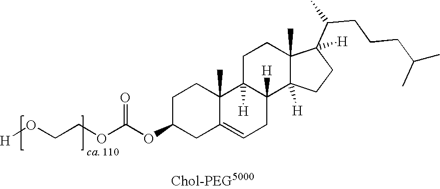

Size determination was performed using dynamic light scattering analysis conducted on a N4 Plus (Beckman Coulter) apparatus at a detection angle of 90°. Mean particle diameters were determined by multimodal fit analysis.

### Example 11

### In Vitro Transfection Efficiency of pDNA-AB Delivery Vehicles

Cells and Culture Conditions. The in vitro transfection efficiency of pDNA-AB nanoparticle systems were evaluated in transient transfection experiments with a variety of mammalian cell lines. The following cell lines were used: HeLa cells derived from a human epithelioid cervical carcinoma; A549 cells derived from a human lung carcinoma and IB-3 cells which are derived from an immortalized primary human culture of bronchial epithelia cells. All cells were grown for transfection in DMEM supplemented with 10% FCS, penicillin at 100 units/mL, and streptomycin at 100 μg/mL. Cells were routinely maintained on plastic tissue culture flasks at 37° C. in a humidified 5% CO₂/95% air atmosphere.

In Vitro Transfection, Luciferase Assay, and Determination of In vitro Cytotoxicity. In vitro transfection experiments were performed and luciferase activity was measured as follows. For each transfection well, Hela, A549 or IB-3 cells were incubated in serum free medium with pDNA-AB nanoparticle systems formulated from DODAG/DOPE 1:1 m/m cationic liposomes and pDNA (1 μg/well, pCMV-Luc) at different lipid:pDNA w/w ratios. After 4 h, each transfection mixture was replaced by a fresh 10% FCS-enriched medium and the cells in each well were incubated again for 24 h at 37° C. in a humidified 5% CO₂/95% air atmosphere. The cells were then lysed and luciferase activity determined. Data for luciferase activity were expressed as relative light units (RLU) per milligram of cell protein, the protein concentration being determined by a standard protein assay (Bio-Rad assay).

In vitro cytotoxicity of the DODAG-derived pDNA-AB nanoparticle systems was quantified by analyzing the total amount of cell protein and by the percentage (%) of LDH released in the medium from damaged cells as an index of cell viability. Cytotoxicity data are expressed as % of LDH release and as concentration of extractable total cell protein in the cell lysate (of fixed volume).

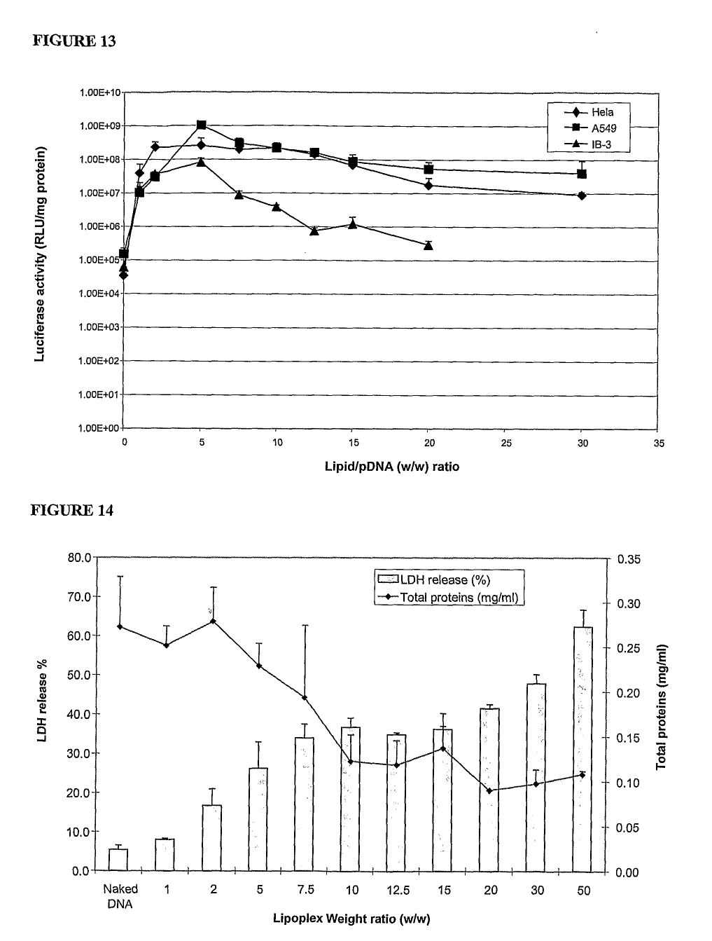

Results. The in vitro transfection efficiencies of pDNA-AB nanoparticles prepared from DODAG/DOPE (1:1, m/m) liposomes and luciferase expression plasmid (pCMV-luc), was examined in Hela, A549 and IB-3 cell lines employing a range of lipid:pDNA ratios (as indicated) and a fixed dose of 1 μg of pCMV-luc per transfection well. The dose response curve (FIG. 13) indicates efficient transfection in all cell lines tested. The optimal lipid:pDNA ratio for transfection, at 24 h post-transfection in Hela and A549 cell lines, appears to be above 5:1 w/w (transfection levels of 10⁸ luciferase RLUs for Hela and A549 were observed up to a 30:1 w/w lipid:pDNA ratio. By contrast, IB-3 cells exhibited a slight decrease in transfection efficiency at the highest lipid:pDNA weight ratios.

Cytotoxicity of pDNA-AB nanoparticles prepared from DODAG/DOPE (1:1, m/m) liposomes and luciferase expression plasmid (pCMV-luc), was examined in Hela cell lines employing a range of lipid:pDNA ratios (as indicated) and a fixed dose of 1 μg of pCMV-luc once again. Toxicity was conveniently quantified using the total amount of cell protein in the cell lysate per well as an index of cell number in parallel with the quantification of LDH release in the medium from damaged cells. As shown (FIG. 14) only a slight decline in total extractable cell protein and similarly a slight increase in LDH release was observed at lipid:pDNA ratios between 0 and 7.5. Substantial toxicities were observed only at very high lipid:pDNA weight ratios. Here, it's worthy to note that for optimal transfection, one needs to balance the efficiency of gene delivery against the cytotoxicity of the vector as cytotoxicity may limit transgene expression. However, our results suggest clearly that pDNA-AB nanoparticles prepared from DODAG/DOPE (1:1) cationic liposomes mediate high transfection activity with low apparent cellular in vitro toxicity.

### Example 12

### In Vivo Transfection Efficiency of pDNA-ABC Delivery Vehicles

Gene Delivery to Mouse Airways and beta-galactosidase Expression in Vivo. Female BALB/c mice (˜30 g body weight) were purchased from Charles River Ltd, UK. Intranasal administration of the lipoplexes was conducted as follow. Each mouse was briefly anesthetized with isoflurane and instilled intranasally with a solution (100 μL/animal) of pDNA-ABC nanoparticles formulated from cationic liposome DODAG/DOPE (1:1, mini) combined with pDNA (pUMVC1-nt-βgal) (lipid:pDNA ratio of 0.5 or 2, w/w) and added Chol-PEG⁵⁰⁰⁰ (Chol-PEG⁵⁰⁰⁰:pDNA ratio of 2, w/w) (Chol-PEG⁵⁰⁰⁰ amounts equivalent to 35 mol % and 15 mol % of total lipid present, respectively). Each animal received a single dose of 25 μg of pUMVC1-nt-βgal pDNA. At 24 h post-instillation, the animals were sacrificed by an i.p.-administered overdose of pentobarbital and the lungs were removed for beta-Galactosidase (βgal) expression analysis. In brief, tissue pieces were placed in a lysis buffer reagent (Roche Diagnostics, UK) and disrupted on ice for about 30 s using an Ultra-Turrax T8 homogenizer. Cells were then lysed by three freeze-thaw cycles, and the clear supernatant was obtained by centrifugation. The βgal concentration was determined using a beta Gal ELISA assay performed according to the manufacturer's instructions (Roche Diagnostics, UK). Enzyme levels were expressed as nanogram of βgal activity per 100 mg of total protein, the protein concentration being determined using the Bio-Rad assay. Results are expressed as mean+/−SEM values. The nonparametric Mann-Whitney test was used throughout when indicated for comparison between mice experimental groups.

Analysis of beta-galactosidase Expression. Left lung lobes from untreated and treated mice with pDNA-ABC nanoparticles from above were fixed for 10 min in 0.5% glutaraldehyde. After an extensive washing with phosphate-buffered saline (PBS), the lobes were incubated for 24 h at 37° C. in an X-Gal solution βgal staining. After completion of βgal staining and extensive washing with PBS, lung tissues were dehydrated in 70%, 95% and 100% ethanol respectively. Clearing of the lung tissues was performed by soaking in a mixture of benzyl benzoate:benzyl alcohol (BBBA) (2:1, v/v). After sufficient clearing, specimens were analysed under a dissection microscope and photographed. Lung tissues were then subjected to histochemical analyses post cryo-section. Briefly, lung tissues were de-cleared by a serial incubation in 100% methanol, methanol in PBS, and PBS respectively. After infiltration with 20% sucrose-containing PBS, the tissue specimens were embedded in a mixture of OCT and 20% sucrose—PBS followed by freezing in pre-cooled isopentane in liquid nitrogen. Cryostat sections of 9 μm were obtained and observed by use of a light microscope before and after eosin counterstaining.

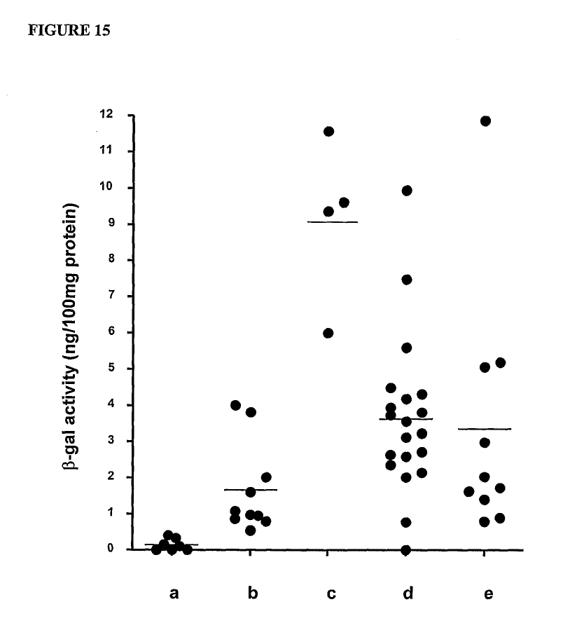

Results. The efficiency of pDNA-ABC nanoparticles formulated from cationic liposome DODAG/DOPE (1:1, m/m) combined with pDNA (pUMVC1-nt-βgal) (lipid:pDNA ratio of 0.5 or 2, w/w) and added Chol-PEG⁵⁰⁰⁰ (Chol-PEG⁵⁰⁰⁰:pDNA ratio of 2, w/w) was investigated by intranasal delivery to the airways of groups of mice. Two lipid:pDNA ratios (0.5:1 and 2:1 w/w) were studied using a fixed dose of 25 μg of pUMVC1-nt-βgal per animal. Mice lungs were removed from untreated and treated mice 24 h post-administration and the presence of βgal activity (ng βgal protein per 100 mg cell protein in lung homogenate) in lungs was analysed by a colorimetric Elisa assay. Transfection comparisons were made with an pDNA-ABC nanoparticle system prepared from CDAN/DOPE (1:1) cationic liposomes and pUMVC1-nt-βgal pDNA (lipid:pDNA ratio 2:1 w/w) with added Chol-PEG⁵⁰⁰⁰ (Chol-PEG⁵⁰⁰⁰:pDNA ratio of 2, w/w), a polymer-based nanoparticle system prepared from invivoJetPEI plus pUMVC1-nt-βgal pDNA (N/P ratio 8), and recombinant adenovirus expressing beta Galactosidase (Adβgal) at a titer of 10¹³. While evaluation of lung lysates of nontreated animals was negative, a high βgal activity was observed with our pDNA-ABC nanoparticles formulated from cationic liposome DODAG/DOPE (1:1, m/m) (FIG. 15). Interestingly, our results also suggested a significant improvement in βgal expression levels of around 5 times higher compared to levels obtained with pDNA-ABC nanoparticles formulated from cationic liposome CDAN/DOPE (1:1, m/m) and more than two times higher compared to levels obtained with invivoJetPEI-DNA and recombinant Adβgal.

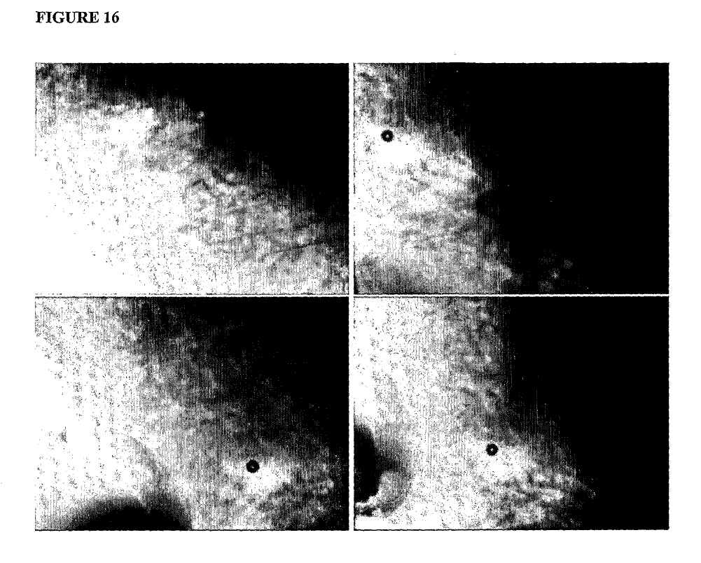

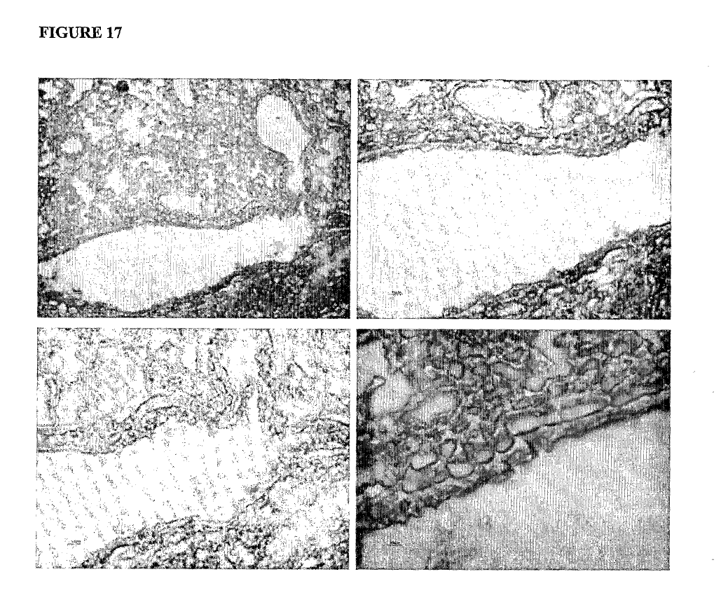

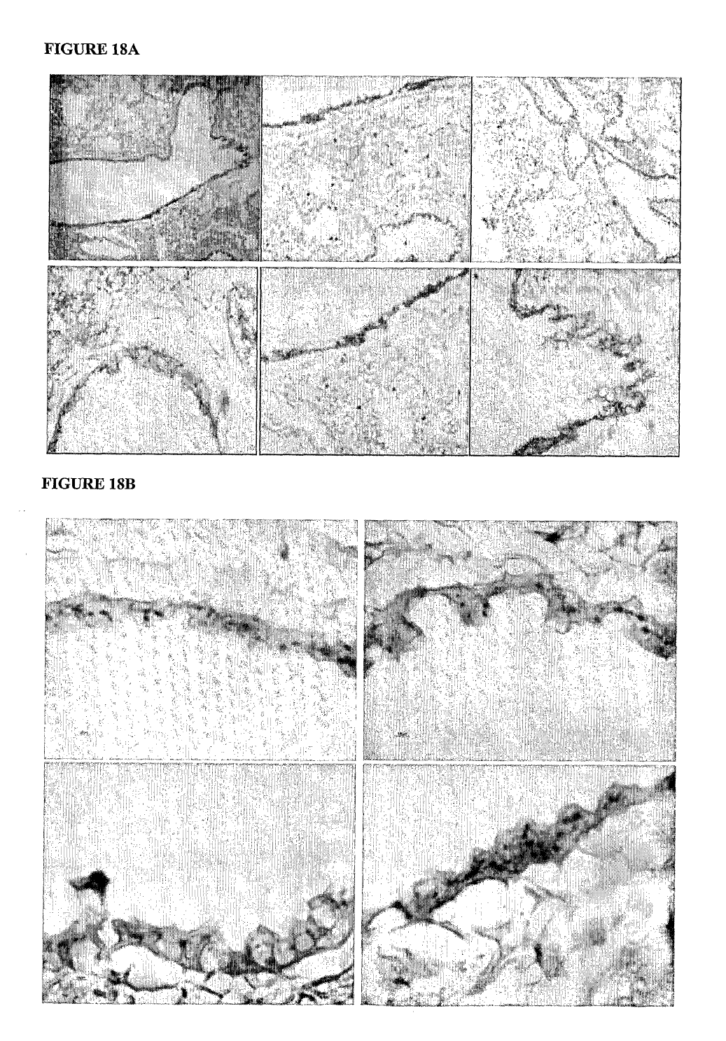

Consistent with the colorimetric data, βgal analysis of the lungs in toto of untreated animals did not show blue staining and no βgal activity was detected. In contrast, lungs of animals instilled with pDNA-ABC nanoparticles formulated from cationic liposome DODAG/DOPE (1:1, m/m) appeared intensely blue with βgal activity localized to the airways and interestingly also in the lung parenchyma (FIG. 16). A variety of different light microscopic histological methods on OCT-PBS embedded sections of lung tissues all suggested that after pDNA complex instillation, the bronchial epithelium of animals were subject to pDNA transfection and intracellular nuclear βgal activity in almost all airway segments evaluated (FIG. 17). Interestingly, lung parenchyma also showed a high net βgal activity indicative of strong transfection in terminal bronchioles and alveoli. In this regard, no nuclear βgal activity was observed in lung sections from untreated mice. Similarly, when counterstained with eosin, no blue staining was observed in either the airway epithelium or the parenchyma lung tissue sections of untreated animals, whereas a substantial nuclear βgal activity was detected in these areas in lung sections from mice treated with pDNA-ABC nanoparticles formulated from cationic liposome DODAG/DOPE (1:1, m/m) (FIGS. 18A and 18B).

Hence such pDNA-ABC nanoparticles formulated from DODAG/DOPE (1:1, m/m) cationic liposomes seem to be a promising candidate for efficient delivery of functional pDNA to lungs and hence for gene therapy applications.

### Example 13

### siRNA Delivery Vehicles for Primary Human T-Lymphocytes

Experimental. Primary T-lymphocytes were collected from heparinised whole blood supplied in blood bags. Blood was diluted 1:1 with DPBS. The blood was mixed thoroughly. Gradient tubes were prepared by adding 15 ml of lymphoprep to a 50 ml tube. With gradient tube held at a sloping angle, slowly & gently a layer of 25 mls of blood/D-PBS mixture was added onto the gradient. Tubes were centrifuged at RT at 2500 rpm for 25 mins with the centrifuge brake at its lowest setting. The buffy coat layer (thin layer between red blood cells and plasma) containing the PBMCs was carefully removed from each tube and washed twice in DPBS. The pellet was resuspended in 50 mls DPBS and cell number was determined. The Miltenyi Biotec protocol for negative selection of CD4+ T cells was followed. Cells were added on a coated 96 well flat bottom tissue culture plate with anti-CD3/anti-CD28 (in a 50 μL DPBS solution/well). Cells were added at 5×10³ cells/well concentration.

A set of six different siRNA-AB and siRNA-ABC nanoparticles was prepared from CDAN 1 or DODAG 5, DOPC and Cholesterol (Chol) with or without the inclusion of DSPE-PEG²⁰⁰⁰. In order to do this, the following cationic liposome systems were prepared first (at approx. 3 mg/ml):
1) DODAG/DOPC/Chol, 20:60:20 mol %
2) DODAG/DOPC/Chol, 50:30:20 mol %
3) DODAG/DOPC/Chol/DOPE-PEG²⁰⁰⁰, 50:30:19:1 mol %
4) CDAN/DOPE, 45:55 mol % (freshly prepared)
5) CDAN/DOPE/DOPE-PEG²⁰⁰⁰, 45:54:1 mol %
6) CDAN/DOPE, 45:55 mol % (stored at 4° C. for 6 months)

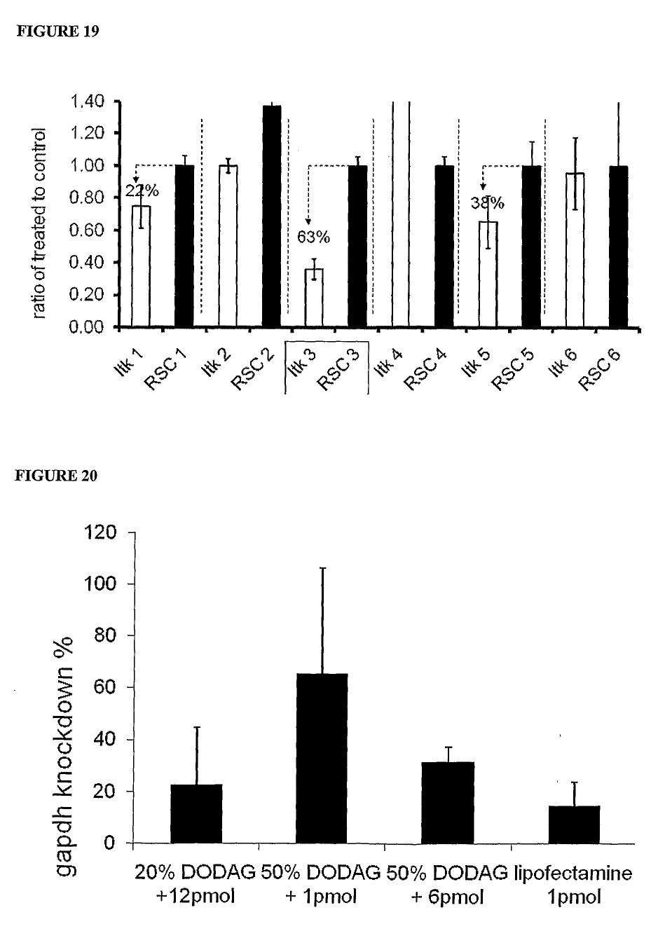

All the cationic liposome formulations above were prepared by dehydration and rehydration of lipid films in deionized water or low ionic strength buffer followed by incubation at ambient to 40° C. for 30 min with mild to strong sonication. Cationic liposomes 1, 2, 4 and 6 were combined with siRNA (lipid:siRNA, ratio 13:1, w/w) to form siRNA-AB nanoparticles 1, 2, 4 and 6 for delivery of siRNA to suspension cells (2 nM/well) (FIG. 19). Cationic liposomes 3 and 5 were combined with siRNA (lipid:siRNA, ratio 13:1, w/w) to form siRNA-ABC nanoparticles 3 and 5 for delivery of siRNA to suspension cells (2 nM/well) (FIG. 19). The siRNA used was either Dharmacon smart pool itk (interleukin tyrosine kinase) or RSC siRNA. Formulations were incubated in Optimem with T-lymphocytes at 37° C. in a shaker incubator at a minimum shaking. After 4 hrs full RPMI medium (+serum+p/s) was added on the cells. Cells were analyzed the next day for gene knockdown using Taqman RT-PCR.

Results. Delivery of siRNA to cells is a key obstacle for siRNA's therapeutic applications. T-cells that actively test their environment do not take up siRNA unless physical or chemical methods can be applied. In this experiment, siRNAs targeted against interleukin-2 inducible T-cell kinase (itk) were used in their “smartpool” anti-itk form. The levels of itk mRNA was determined 1d post delivery of anti-itk siRNA (FIG. 19). Data suggest that siRNA-ABC nanoparticles 3 and 5 exhibit the highest functional delivery of siRNA with the highest corresponding knockdown efficiency. In all cases, DODAG-containing siRNA-AB and siRNA-ABC nanoparticles are superior to their CDAN-containing counterparts for functional delivery of siRNA to T cells (i.e. formulation 3 is better than 5; formulation 1 out performs 6).

### Example 14

### siRNA Delivery in Jurkat Cells Using DODAG Containing siRNA-ABC Nanoparticles

Experimental. Jurkat cells (derived from a human acute T-cell leukemia line) are used extensively in studies on T cell signaling and cancer drug development. Here, Jurkat Cells (clone E6-1, ATCC) were seeded at 100,000 cells/well seeding density. Jurkat cells were cultured in suspension in RPMI+ fetal bovine serum. Two different siRNA-ABC nanoparticles were prepared from DODAG 5, DOPC and Cholesterol (Chol) with DSPE-PEG²⁰⁰⁰. In order to do this, the following cationic liposome systems were prepared first (at approx. 3 mg/ml):
1) DODAG/DOPC/Chol/DOPE-PEG²⁰⁰⁰, 20:60:19:1 mol %
2) DODAG/DOPC/Chol/DOPE-PEG²⁰⁰⁰, 50:30:19:1 mol %

Both the cationic liposome formulations above were prepared by dehydration and rehydration of lipid films in deionized water or low ionic strength buffer followed by incubation at ambient to 40° C. for 30 min with mild to strong sonication. Cationic liposomes 1 and 2 were combined with siRNA (lipid:siRNA, ratio 13:1, w/w) to form siRNA ABC nanoparticles for delivery of siRNA to suspension cells at different concentrations (1, 6 and 12 nM/well; 1, 6 and 12 pmol/well respectively). The siRNAs used were either anti-human Glyceraldehyde-3-phosphate dehydrogenase (anti-GAPDH, RNA+) or the corresponding RSC control (RNA−). Before siRNA delivery, Jurkat cells were transferred to Optimem and then cells were incubated for 4 h in the presence of either of the siRNA ABC nanoparticles described above, prior to the addition of full media. Cell lysis was performed 24 h after siRNA delivery and lysate was analysed for GAPDH content using an Ambion assay (KD-alert) and a Varioskan fluorescence plate reader. Gene knockdown was calculated as:

$$\% knockdown = 100 - ( 100 \times \frac{Δ {fluorescence}_{GAPDH}}{Δ {fluorescence}_{Neg}} )$$

Results. Jurkat cells are derived from a human acute T-cell leukemia line and are used extensively in the study of T cell signaling and cancer drug development. In this experiment (FIG. 20) Jurkat cells were incubated with various DODAG-containing siRNA-ABC nanoparticles. In general, efficient knockdown will cause a more dramatic reduction in target mRNA levels than in target protein levels (ambion KD-alert manual FIG. 1 comparison of KD-alert and mRNA detection by RT-PCR). This is due probably because protein knockdown is influenced by the rates of protein synthesis and turnover, in addition to the rates of target mRNA synthesis and turnover. For housekeeping genes such as GAPDH, the rate of cell division and concomitant synthesis of the housekeeping protein during the transfection experiment also have an impact on knockdown levels. Levels of GAPDH in cells post siRNA-ABC nanoparticle-mediated siRNA delivery are shown (Table 2). The most effective knock down was achieved with the siRNA-ABC nanoparticle system prepared with 50 mol % of DODAG (using cationic liposome 2, Example 13) at the lowest delivered dose of siRNA (1 pmol/well) (see FIG. 20). This was also the most effective siRNA-ABC nanoparticle formulation in Example 13. By way of benchmark, lipofectAMINE2000 (Invitrogen) failed to mediate functional siRNA delivery to Jurkat cells under any circumstances. This may be due to the relatively high cytotoxicity of lipofectamine formulations.

TABLE 2
Levels of GAPDH after application of DODAG nanoparticles in Jurkat cell
suspensions.

| | DODAG 20% + | DODAG 50% + | DODAG50% + | lipofect amine + |
|---|---|---|---|---|
| GAPDH | 12 pmol | 1 pmol | 6 pmol | 1 pmol |
| siRNA+ | 0.042 +/− 0.01 | 0.020 +/− 0.019 | 0.030 +/− 0.002 | 0.037 +/− 0.003 |
| siRNA − | 0.053 +/− 0.001 | 0.051 +/− 0.004 | 0.042 +/− 0.009 | 0.042 +/− 0.013 |

### Example 15

### Engineering DODAG-Containing siRNA-ABC Nanoparticle Formulations

Incorporation of DODAG at various molar ratios can provide stable particles of various size and charges (zeta potential). This may be used to engineer particles for delivery of siRNA to various types of cells and tissues, with a particular interest in tumour delivery.

Experimental. Two different siRNA-ABC nanoparticles were prepared from DODAG 5, DOPC and Cholesterol (Chol) with DSPE-PEG²⁰⁰⁰. In order to do this, the following cationic liposome systems were prepared first (at approx. 3 mg/ml):
1) DODAG/DOPC/Chol/DOPE-PEG²⁰⁰⁰, 20:60:19:1 mol %
2) DODAG/DOPC/Chol/DOPE-PEG²⁰⁰⁰, 50:30:19:1 mol %

Both the cationic liposome formulations above were prepared by dehydration and rehydration of lipid films in deionized water or low ionic strength buffer followed by incubation at ambient to 40° C. for 30 min with mild to strong sonication. Cationic liposomes 1 and 2 were combined with siRNA (N/P ratios 16 to 1.5) to form siRNA-ABC nanoparticles for size and zeta analysis. When 20 mol % DODAG is used, N/P ratios of 1.5, 2, 4 and 8 correspond with lipid:siRNA ratios of 13:1, 16:1, 32:1 and 64:1 w/w, etc. When 50 mol % DODAG is used, N/P ratios 1.5, 2, 4 and 8 correspond with lipid:siRNA ratios of 4.5:1, 6.5:1, 13:1 and 26:1 w/w, etc. Size was measured with Coulter N4 Photon Correlation spectrometer. Charge (i.e. zeta potential) was measured with a Malvern Zetasizer Nano ZS. All siRNA loading efficiencies of siRNA-ABC nanoparticles were assessed using a fluorimetric assay. YOYO-1 nucleic acid probe was used at a concentration of 2.5 μM. The various DODAG-containing siRNA-ABC nanoparticles formulated here were incubated with YOYO-1 solution for 10 min at RT before measurement of their fluorescent intensity at 509 nm. YOYO-1 is a DNA or RNA intercalator; hence the higher the fluorescence intensity the higher the amount of non-encapsulated siRNA.

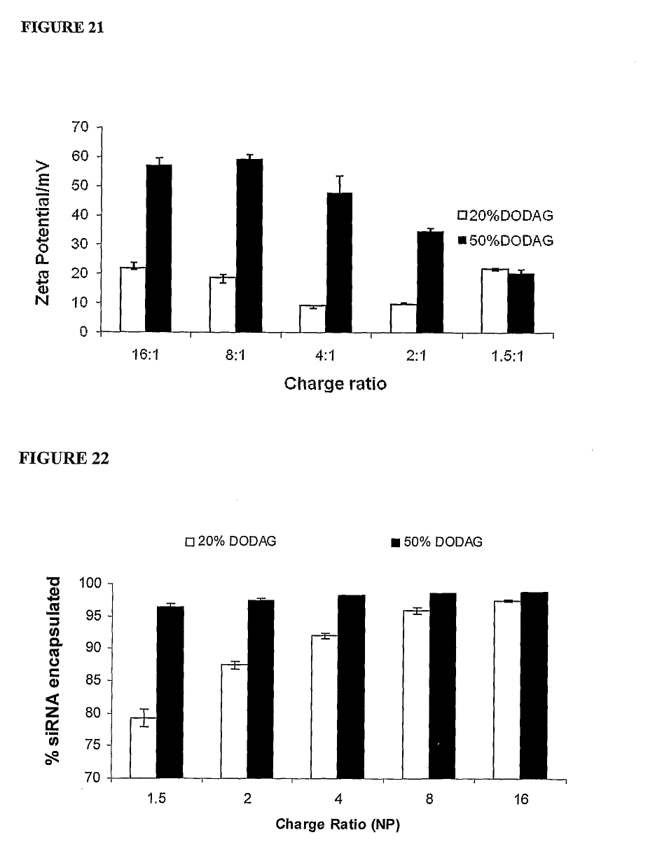

Results. Control of nanoparticle size and charge is highly desirable for efficient functional delivery of therapeutic nucleic acids. Data (Table 3) illustrates that DODAG-containing siRNA-ABC nanoparticles can be formulated with siRNA over the complete range of N/P ratios without a substantial effect on size except at N/P 1.5 (lipid:siRNA, 4.5:1, w/w) with respect to the siRNA-ABC nanoparticle systems prepared from 50 mol % DODAG 5. The lower N/P systems would be expected to be more useful for prolonged circulation in vivo and delivery to tumour. Also, the inclusion of DSPE-PEG²⁰⁰⁰ allows for the generation of formulations with controlled charge (zeta potential) as well as size (FIG. 21). Even though only 1 mol % of DSPE-PEG²⁰⁰⁰ is included in the DODAG-containing siRNA-ABC nanoparticles, the zetapotential may be reduced as low as +9 mV (siRNA-ABC nanoparticles formulated with 20 mol % DODAG; N/P ratios, 2:1 and 4:1; lipid:siRNA ratio, 16:1 and 32:1 w/w, respectively). Such particles should be ideal for improved biological stability and are less preferred by the immune system including the reticulo-endothelial system (RES). The correlation between N/P ratio and siRNA encapsulation efficiency is shown (FIG. 22). Acceptable levels of encapsulation efficiency above 90% are found with siRNA-ABC nanoparticles prepared from both 50 mol % and 20 mol % DODAG. In this respect, all siRNA-ABC nanoparticles prepared from 50% DODAG were 95% efficient at encapsulation irrespective of lipid:siRNA ratios. siRNA-ABC nanoparticles prepared from 20 mol % DODAG were 95% efficient for encapsulation of siRNA with an N/P ratio 8:1 (lipid:siRNA ratio of 64:1 w/w or greater). At the lower N/P ratios, 2:1 and 4:1 (lipid:siRNA ratios, 16:1 and 32:1 w/w respectively) encapsulation efficiency still appears to be 85% or better.

TABLE 3
Effect of nanoparticle/siRNA charge ratio on nanoparticle size

| N/P ratio | 1.5/1 | 2/1 | 4/1 | 8/1 | 16/1 |
|---|---|---|---|---|---|
| 20% DODAG size (nm) | 123.7 | 112.3 | 100.8 | 96.7 | 96.2 |
| 50% DODAG size (nm) | 318.65 | 121.5 | 108.4 | 131.4 | 141.1 |

### Example 16

### Effect of DODAG Formulation on GAPDH siRNA Uptake by DU145 Prostate Cancer Cells

Experimental: Two different siRNA-ABC nanoparticles were prepared from DODAG 5, DOPC and Cholesterol (Chol) with DSPE-PEG²⁰⁰⁰. In order to do this, the following cationic liposome systems were prepared first (at approx. 3 mg/ml):
1) DODAG/DOPC/Chol/DOPE-PEG²⁰⁰⁰, 20:60:19:1 mol %
2) DODAG/DOPC/Chol/DOPE-PEG²⁰⁰⁰, 50:30:19:1 mol %

Both the cationic liposome formulations above were prepared by dehydration and rehydration of lipid films in deionized water or low ionic strength buffer followed by incubation at ambient to 40° C. for 30 min with mild to strong sonication. Cationic liposomes 1 and 2 were combined with FAM-labelled siRNA in a variety of N/P ratios (as for Example 15) and used to treat DU145 cancer cells (ATCC) (final siRNA concentration 15 nM/well). These cells were cultured in DMEM-FCS culture media for 1 week before siRNA transfection experiments. Cells reaching confluency 80% were used for siRNA delivery experiments. The siRNA-ABC nanoparticles were incubated in the presence of DU145 cells for 2 or 4 h, after which they were trypsinized and prepared for FACS analysis.

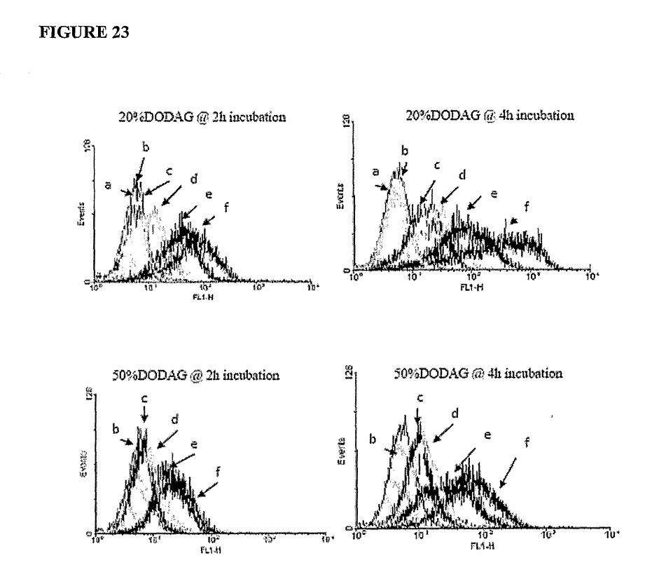

Results. FACS studies (FIG. 23) showed that DODAG containing siRNA-ABC nanoparticles do associate with and enter cells. The higher the N/P ratio, the better the cell associated fluorescence with adherent DU145 cells. Cell uptake is time dependent as cell associated fluorescence appears higher after 4 h of incubation compared to cell associated fluorescence at 2 h of incubation. Free FAM-siRNA shows no cell-associated fluorescence indicating that siRNA requires a carrier for efficient cellular delivery. The siRNA-ABC nanoparticles formulated with 20 mol % DODAG and N/P 4 (lipid:siRNA ratio 32:1 w/w) appears to be a particularly useful system for effective cell entry at 4 h. This same siRNA-ABC nanoparticle system has an optimal low zeta potential (FIG. 21), and retains very high encapsulation efficiency for siRNA (FIG. 22). Hence this is good foundation for in vivo delivery of siRNA.

### Example 17

### Biodistribution of DODAG Containing siRNA-ABC Nanoparticles in Xenograft Mice

Experimental. Balb/C nude immunocompetent mice 6-8 weeks old were used to grow tumours and prepare the xenograft model. IGROV ovarian cancer cells were injected s.c. (5×10⁶/ml in PBS) at the right flank. Tumours were allowed to grow for 2 weeks. If MRI imaging is required then mice were anaesthetized with an isoflurane/O₂ mix and placed into a quadrature ¹H volume coil and positioned into the magnet. Baseline scans were obtained and then the mice were injected intravenously via lateral tail vein with either a 200 μL liposome solution (HEPES (20 mM, NaCl-135 mM, pH 6.5)) and imaged at 4.7 T (spin echo sequence: TR=400-2800 ms, TE=10 ms, FOV=45×45 cm², averages: 1, matrix size: 256×128, thickness: 2.0 mm, and 20 slices).

A single siRNA-ABC nanoparticle formulation was prepared from DODAG 5, DOPC and Cholesterol (Chol) with DSPE-PEG²⁰⁰⁰. In order to do this, the following cationic liposome system was prepared first (at approx. 3 mg/ml):
DODAG/DOPC/Chol/DOPE-PEG²⁰⁰⁰/DOPE-Rho/Gd-DOTA-DSA, 20:22:20:7:1:30 mol %

The cationic liposome formulations above were prepared by dehydration and rehydration of lipid films in deionized water or low ionic strength buffer followed by incubation at ambient to 40° C. for 30 min with mild to strong sonication. The cationic liposomes formulation was then combined with FAM-labelled siRNA (N/P 4:1, lipid:siRNA, ratios 32:1 w/w) (final siRNA concentration 10 μg/ml). Formulation was administered at 190 ng of FAM-siRNA/mouse or approx. 10 μg/kg by tail vein injection ( 1/100^{th} of normal dose of 1 mg/kg used in other in vivo studies).

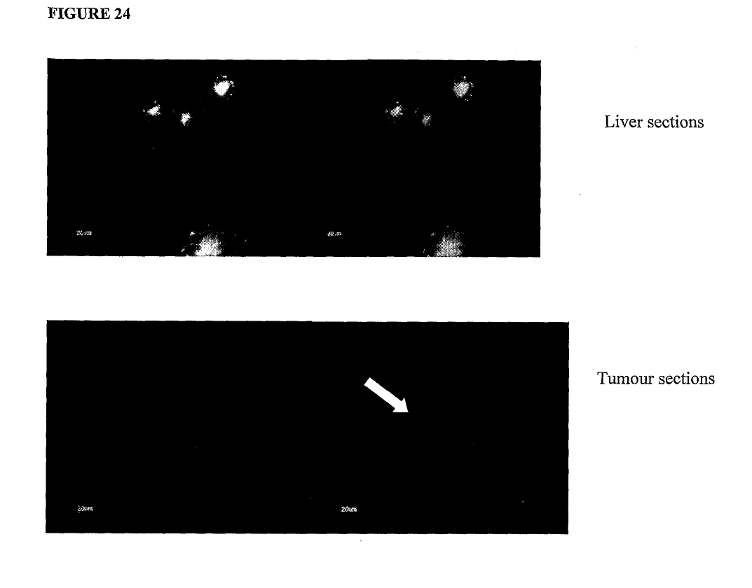

Results. MRI was used to monitor for siRNA-ABC nanoparticle accumulation in animal tumours at 0, 14 and 24 h post i.v. injection (according to the procedures described in Kamaly, N. et al., Bimodal Paramagnetic and Fluorescent Liposomes for Cellular and Tumor Magnetic Resonance Imaging. Bioconj. Chem. (2007) in press). Animals were sacrificed at 24 h post administration and organs were prepared for microscopy (sectioning). Liver sections showed nanoparticle presence (FIG. 24 upper panel) 24 h after the injection. Kupffer cells appear to keep most of the nanoparticles that carry the siRNA. The left hand image corresponds to rhodamine labeled lipid and the right hand image corresponds to FAM-labeled siRNA. Tumour sections (FIG. 24 lower panel) showed tumour nanoparticle uptake at 24 h post injection. FAM-siRNA (right hand image) appears to be co-localised with Rhodamine signal indicating that the 20 mol % DODAG-containing siRNA-ABC nanoparticle used here are able to enter tumour tissue and deliver siRNA to tumour cells up to 24 h post administration. This 20 mol % DODAG-containing siRNA-ABC nanoparticle appears to be a very good starting point for siRNA (and even pDNA) therapeutic approaches to the treatment of cancer.

All publications mentioned in the above specification are herein incorporated by reference. Various modifications and variations of the described methods and system of the invention will be apparent to those skilled in the art without departing from the spirit of the invention. Although the invention has been described in connection with specific preferred embodiments, it should be understood that the invention as claimed should not be unduly limited to such specific embodiments. Indeed, various modifications of the described modes for carrying out the invention which are straightforward to those skilled in lipid chemistry and molecular biology or related fields are intended to be within the scope of the following claims.

1. A lipid of the formula (I)
R₃R₄N—[Y]_{q}—(CₚH₂ₚ)—X-Linker-NR₁R₂ (I)
wherein
R₃ and R₄ are independently selected from H and hydrocarbyl groups;
q is an integer selected from 1 to 10; Y represents a group (CₙH₂ₙ)NR₅, wherein (i) when q is 1, n is 2, or (ii) when q is greater than 1, each Y may be the same or different and each n is an integer independently selected from 1 to 10, with the proviso that for at least one unit Y, n is 2, and (iii) each R₅ is independently selected from H and hydrocarbyl groups;
p is an integer selected from 1 to 10;
X is an optional group selected from —NR₇—, —C(═O)NR₈—, —NR₉C(═O)—, —C(═O)—, —O—, and —NR₁₀C(═O)O—, wherein each R₇, R₈, R₉ and Ri₀ is independently selected from H and hydrocarbyl groups,
Linker is an optional group selected from amino acid residues, peptide residues and groups of the formulae —(OCH₂CH₂)₁₋₁₀, —NR₆(CᵥH₂ᵥ)—C(═O), wherein R₆ is H or a hydrocarbyl group and v is an integer selected from 1 to 11, and —C(═O)(CH₂)₀₋₁₀—CH₂—C(═O)—;
R₁ is selected from acyclic groups having from 4 to 30 carbon atoms; and
R₂ is selected from H and acyclic groups having from 4 to 30 carbon atoms.

2. A lipid according to claim 1 wherein when q is greater than 1, each n is an integer independently selected from 2 to 4.

3. A lipid according to claim 1 wherein q is 2.

4. A lipid according to claim 1 wherein p is 2 or 3.

5. A lipid according to claim 1 wherein each R₃, R₄ and R₅ is independently selected from H and C₁₋₃ alkyl groups.

6. A lipid according to claim 1 wherein each R₃, R₄ and R₅ is independently selected from H and methyl.

7. A lipid according to claim 1 wherein each R₃, R₄ and R₅ are the same.

8. A lipid according to claim 1 selected from lipids of the formulae
H₂N—(CH₂)₂—HN—(CH₂)₂—X-Linker-NR₁R₂
H₂N—(CH₂)₃—HN—(CH₂)₂—X-Linker-NR₁R₂
H₂N—(CH₂)₄—HN—(CH₂)₂—X-Linker-NR₁R₂
H₂N—(CH₂)₂—HN—(CH₂)₃—X-Linker-NR₁R₂
H₂N—(CH₂)₂—HN—(CH₂)₃—HN—(CH₂)₂—X-Linker-NR₁R₂
H₂N—(CH₂)₂—HN—(CH₂)₃—HN—(CH₂)₃—X-Linker-NR₁R₂
H₂N—(CH₂)₂—HN—(CH₂)₄—HN—(CH₂)₂—X-Linker-NR₁R₂
H₂N—(CH₂)₃—HN—(CH₂)₂—HN—(CH₂)₂—X-Linker-NR₁R₂
H₂N—(CH₂)₃—HN—(CH₂)₄—HN—(CH₂)₂—X-Linker-NR₁R₂
H₂N—(CH₂)₂—HN—(CH₂)₃—HN—(CH₂)₄—HN—(CH₂)₃—X-Linker-NR₁R₂
Me₂N—(CH₂)₂—NMe—(CH₂)₂—X-Linker-NR₁R₂
Me₂N—(CH₂)₃—NMe—(CH₂)₂—X-Linker-NR₁R₂
Me₂N—(CH₂)₄—NMe—(CH₂)₂—X-Linker-NR₁R₂
Me₂N—(CH₂)₂—NMe—(CH₂)₃—X-Linker-NR₁R₂
Me₂N—(CH₂)₂—NMe—(CH₂)₃—NMe—(CH₂)₂—X-Linker-NR₁R₂
Me₂N—(CH₂)₂—NMe—(CH₂)₃—NMe—(CH₂)₃—X-Linker-NR₁R₂
Me₂N—(CH₂)₂—NMe—(CH₂)₄—NMe—(CH₂)₂—X-Linker-NR₁R₂
Me₂N—(CH₂)₃—NMe—(CH₂)₂—NMe—(CH₂)₂—X-Linker-NR₁R₂
Me₂N—(CH₂)₃—NMe—(CH₂)₄—NMe—(CH₂)₂—X-Linker-NR₁R₂
Me₂N—(CH₂)₂—NMe—(CH₂)₃—NMe—(CH₂)₄—NMe—(CH₂)₃—X-Linker-NR₁R₂

9. A lipid according to claim 1 wherein R₁ and R₂ are each independently selected from alkyl, alkenyl and alkynyl groups having from 4 to 30 carbon atoms.

10. A lipid according to claim 1 wherein R₁ and R₂ are each independently selected from alkyl groups having 18 carbon atoms.

11. A lipid according to claim 1 wherein X is present.

12. A lipid according to claim 11 wherein X is —C(═O)—.

13. A lipid according to claim 1 wherein Linker is an amino acid residue, peptide residue or group of the formula —NR₆—(CᵥH₂ᵥ)—C(═O)—.

14. A lipid according to claim 13 wherein Linker is an amino acid residue.

15. A lipid according to claim 1 of the formula
H₂N—(CH₂)₂—HN—(CH₂)₃—HN—(CH₂)₂—C(═O)NH—CH₂—C(═O)—N[(CH₂)₁₇CH₃]₂ or.
Me₂N—(CH₂)₂—NMe—(CH₂)₃—NMe—(CH₂)₂—C(═O)NH—CH₂—C(═O)—N[(CH₂)₁₇CH₃]₂.

16. (canceled)

17. A micelle formed from or comprising a lipid according to claim 1.

18. A liposome formed from or comprising a lipid according to claim 1.

19. A delivery vehicle comprising (i) a lipid according to claim 1 and (ii) one or more agents.

20. A delivery vehicle according to claim 19 comprising an ABCD nanoparticle, wherein A is one or more agents, B comprises the lipid, C is an optional component comprising polymers having stealth/biocompatibility properties and D is an optional component comprising a targeting moiety.

21. A delivery vehicle according to claim 19, wherein the one or more agents comprises a therapeutic or diagnostic agent.

22. A delivery vehicle according to claim 21, wherein the therapeutic agent comprises siRNA.

23. A method of treating a subject having a disorder, a condition or a disease or diagnosing the subject as suffering from the disorder, the condition or the disease which comprises using the lipid according to claim 1.

24. The method of claim 23 wherein the method comprises delivering the lipid to the subject or using the lipid for imaging.

25. The method of claim 23, wherein the disorder is a genetic disorder or the disease, disorder or condition is associated with liver disease and/or liver damage, and cancer.

26-27. (canceled)

28. A method for delivering one or more agents to one or more cells comprising administering a composition comprising a) a lipid according to claim 1 and b) one or more agents.

29-32. (canceled)

33. A composition comprising a lipid according to claim 1, further comprising additional lipids, sterols, fatty acids, dicetyl phosphate, cholesterol hemisuccinate, preservatives PEGylated derivatives of phosphatidylethanolamine, conjugates of sugars and hydrophobic components, diagnostic markers, contrasting media, imaging aids and/or amphiphilic compounds.

34. The composition according to claim 33, wherein the additional lipid is a neutral lipid, sterol lipid, fluorescent lipid, magnetic resonance imaging lipid, nuclear magnetic resonance imaging lipid, electron microscopy and image processing lipid, electron spin resonance lipid or radioimaging lipid.

35-36. (canceled)
